SEQUENCE LISTING (171 sequences)

SEQ ID NO: 1 - DNA, 505 bp, Homo sapiens
  source 1..505: mol_type = other DNA
acatttcccttgccccttgcttttatgcttttactgttatttcttagccctgaactgagttgcgagactcccaggtcctagtctcagatctgccacttttcctatttatacaaggaaaagcaaaatcccagtaaaggacaaggaaaaatcaaacatgaagcctcccggccttctctgagagccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagtcatgtgttctgaggaaccaagaagcgagcttactcggtccatcagtggtttaattgttgggcctttctctgcaggtcttattttttagatgaaaagtcaggatacatggtaaaggtcagtgcttggggagattatggtgtca

SEQ ID NO: 2 - DNA, 251 bp, Homo sapiens
  source 1..251: mol_type = other DNA
gcacccgagatagaaggacccattgttttctaaaactagttaccactcttccctttatcctttcttttgtaccttttagcgtttgcttaatcagaatattttaatccctaatgtggtgactcggtgtaatttatctcattaagcctctcctattttctcctgtttacatgactaatattattgtgttttatagagcttataccaacaaatcactcctttgtcccattgtttcagctccaggccaaaaacca

SEQ ID NO: 3 - DNA, 946 bp, Homo sapiens
  source 1..946: mol_type = other DNA
gtccagatgctgttgccgtggtaatactaaatcgtgtccaaagaggaggctgagattcctttttagttaaccttgtccacatgatttgactatccagcaggcttggattcccaggccccagagacttcgggactgttttccagcacttgaatgtggtatataagtgctgatatcaaaagggatatgtaggaaagacaagattataggcagggcattgtctgaccaggacgaggcccgaagcaaaagtctttgtttgtctctggcacctcttgataattgttgttttactcctagaacccacccactgatccccatccatccctagtaccagtcacggctgctctagcatttcggccttgtcaactactgcaggccctgatcaaggaggcctctgttcatggggtaaggttaaggggtgggttctgggaggagggtaagactgtagtgcagattcgagacctagggaggtggggccagtttctcactcaatgagcctgcctgttgtttagctctattgtatagggttcttctatctctccctttgtcagctggattcaagggacaggctgggaattgtgcagggtacaattggtctctttctaccattcctttcgatgaaagcccttcctctccttcctttgatgagttcccagattcacactgaatttccatcatggagctggtccttgaggcgggcccagggcatgggaaaggagggagggagggttgggagctcttggtgcctgcttaaagagacagcatcaggatcaacacagtcccttgatgaacctggatctgtgtaagtcacattggttctgttgatctctgtcagaccgctgtgaagagcgctggtgagaagggcttaaagttggagtatcaactatcacagattgattagacttgttttgggagtttgtatacaaatgtagccaaccatgacatgatc

SEQ ID NO: 4 - DNA, 664 bp, Mus musculus
  source 1..664: mol_type = other DNA
ctggccttaggtctctggggccccagcagccccatgtctgcctccttaaatcggccttgaggttaagtgtgtctctgtctgtctgtctgtctctctgtctctctccagctctgctgaggaagtactaggctctcctcagggcctcgccgcccacagtaacaggtcactcagagcccacagtaacaggtcactcagagcccacagtaacaggtcactcagagtggcactgctttgctcttgctgtttgtaagagaaaaacacgctaagcttcggagcctggcaccaccaggtttggccactcttgattatttcccttgttcccacccctcttctcttgccctcaagtcctgaacccccttcttatcaccctcccccagcccagctgctgcatgtgccaacccctacccagcagaaaaggaaggtcttggaaggggatcaatggaggttctgtgtgggcttgcagctgaaatggccctgcacacagccacagatgggcctctgctccccctccacatttctgcatcaacaaagcgtgaccttgtttagatgacagtcgccccatgtctcccccatgacaatgtttgccttgcttttgcctcctagtgggctgcctcaggacatctctgttgcctgagactggaaaagggtgatgggatgggaagca

SEQ ID NO: 5 - DNA, 304 bp, Homo sapiens
  source 1..304: mol_type = other DNA
caccactctgtgcttagaaccaagagctaggagagcaagggagcctgggactggcacttttcagctagggggagctggtggtggcagggcagagaggcccattgcgcagctggtaacaggctgtgcctggccaggcctcccagctgcccctctgtctcaggcccttattgcacacaacatccagcccagccccctacgcagctgccttcctcatgacacagccctgtgttgtcctgttggccactgaccactgtgcctggggaagggccaggccttcctgggaggaggaactcgagagcctgtg

SEQ ID NO: 6 - DNA, 436 bp, Homo sapiens
  source 1..436: mol_type = other DNA
gagggtctggccgtgagtgtgggagagtccacactgggcgaggagggagggcggggctttgaacatcagacctcagggtgggtggcctgagctgccagtggggccaccagtagccacaagcaaatatctccaccaacaaggcccgtctgtctcgggacaagcatgtctttgtggcgcttgctccccttctgtcctcccccacagctggccctgtccgcccgtgcccagtcaggccccgcagaacccaagaggggcttcccatgccatttaggaagggctgtctctttaaaaaaaaaaaaaattacaaaacggttgatttcccaagaggttgacacagcagcacgtacagtaagatgttcctcacacctgcggggatgccatggtcccagcccggggcacccgttacccaggaaggtggtgagagtggtgctgggaa

SEQ ID NO: 7 - DNA, 394 bp, Homo sapiens
  source 1..394: mol_type = other DNA
gcgagtccaggggctctaccccctcttctgccctccttcccctccctccagggtggttcagtactcgctgggatgggggactctaacccaagtcctccccgcacccatggttctagtcacatctgtcaaagggatccaggcaagaacaaaggcctagtgtgcctcggacaaaggagaaaggtcccgccagccctcacccctccctgggccctgcgtctgccccctcgggaccgccaccatggaaaccccctcccacactcactaatagcccccgtgggtgcccaggacctgagccatcgccagggcaacagggctggttccgacacaggcgtgtgtcccgcagtgtccattcccaagtgccctcccttcgatggggaacacggcaggactccat

SEQ ID NO: 8 - DNA, 300 bp, Mus musculus
  source 1..300: mol_type = other DNA
tcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaac

SEQ ID NO: 9 - DNA, 900 bp, Synthetic construct
  source 1..900: mol_type = other DNA
tcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaactcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaactcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaac

SEQ ID NO: 10 - DNA, 158 bp, Mus musculus
  source 1..158: mol_type = other DNA
aatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgta

SEQ ID NO: 11 - DNA, 474 bp, Synthetic construct
  source 1..474: mol_type = other DNA
aatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgtaaatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgtaaatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgta

SEQ ID NO: 12 - DNA, 119 bp, Homo sapiens
  source 1..119: mol_type = other DNA
gtaacaggctgtgcctggccaggcctcccagctgcccctctgtctcaggcccttattgcacacaacatccagcccagccccctacgcagctgccttcctcatgacacagccctgtgttg

SEQ ID NO: 13 - DNA, 357 bp, Synthetic construct
  source 1..357: mol_type = other DNA
gtaacaggctgtgcctggccaggcctcccagctgcccctctgtctcaggcccttattgcacacaacatccagcccagccccctacgcagctgccttcctcatgacacagccctgtgttggtaacaggctgtgcctggccaggcctcccagctgcccctctgtctcaggcccttattgcacacaacatccagcccagccccctacgcagctgccttcctcatgacacagccctgtgttggtaacaggctgtgcctggccaggcctcccagctgcccctctgtctcaggcccttattgcacacaacatccagcccagccccctacgcagctgccttcctcatgacacagccctgtgttg

SEQ ID NO: 14 - DNA, 184 bp, Homo sapiens
  source 1..184: mol_type = other DNA
agccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagt

SEQ ID NO: 15 - DNA, 552 bp, Synthetic construct
  source 1..552: mol_type = other DNA
agccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagtagccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagtagccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagt

SEQ ID NO: 16 - DNA, 100 bp, Homo sapiens
  source 1..100: mol_type = other DNA
cagaatattttaatccctaatgtggtgactcggtgtaatttatctcattaagcctctcctattttctcctgtttacatgactaatattattgtgttttat

SEQ ID NO: 17 - DNA, 300 bp, Synthetic construct
  source 1..300: mol_type = other DNA
cagaatattttaatccctaatgtggtgactcggtgtaatttatctcattaagcctctcctattttctcctgtttacatgactaatattattgtgttttatcagaatattttaatccctaatgtggtgactcggtgtaatttatctcattaagcctctcctattttctcctgtttacatgactaatattattgtgttttatcagaatattttaatccctaatgtggtgactcggtgtaatttatctcattaagcctctcctattttctcctgtttacatgactaatattattgtgttttat

SEQ ID NO: 18 - DNA, 648 bp, Homo sapiens
  source 1..648: mol_type = other DNA
agcaagttttctacctgggatgcccccaagggtagcaggccatacttcctaagtgtgcatgttggtggggagggagggtatcaaggagtatgtccctggagaagggagaaaggagggggcgtgggaaggggcctgtggaacgcacgtgtctcatttcctctttcggaatgtccgctgtgggccccaacaaaggaacttcggttggaactggggtgaggaagcccccgaagatcaacttctcttcctttaacatttggctctgctgagggccctggggttggaacacccaccttcttagaaagaaactgaaaggccttaggagaaaccccgccacttccccttcctcggtaagaggaacttggcgttgccctgacaggaaagtcttggtggggcttctcggaagccgccaccgctgcactgctgagcaaaaccacaggtcaggggccaggccgggggccaggctagtgaggactggccattgataaagtcacaggctccatctaagcccatcggcagaactggggagctgccaacatttcctcttccagcaccgcctgccaactcctgtttgggagccaccaacccctcagctctgccaatgggtccctccccactcacaccagccctccaaccaggatcgttctgtgt

SEQ ID NO: 19 - DNA, 555 bp, Homo sapiens
  source 1..555: mol_type = other DNA
gggtgattctcccctgcctctcccgggatctttgccaagctcgtcctgttcagcaccaaagacagctcttgggtgccgccttcctggcccaccacccccttgggtgtgggtggatggtaccacctcactcaacatgcttgacgtggactaggcacacctgggtggagcccctcagcatgctgtgctctgcccaggcaataaccctggcaggagtgggcagcccttagacgggagttaggtcccagcaggcatcaagagggtgagagccactccttactgagtgaggggacccataccaactgccttggcctgggcttccttatgaggtctccagcacctcagctgatctgaaactgaggggcaaagaggaaacagaaactggccaggggccctagaacagaaatgcagaacctgaaaccaaatgtagaacagaaagcctgagaaccagctacgcccatgagctgcagacccatgggctgagaaaccagggactggggtgccagggaggggtgggagagcctgggagtagccacacagcactaggtcccaatgctt

SEQ ID NO: 20 - DNA, 478 bp, Homo sapiens
  source 1..478: mol_type = other DNA
tctgatggatctgggcaaagtcaattgaaaaccttctggaaaaaaattcaccattttagatgccattaagaacattcatgattcatcagagggggtcaaaatatcaacattaacacgagttcgggagaagctgattctgattctcatggatgactttgagtggctcaaggcttcaggggaggaagtgactgcggatgtgtggaaatagagaaccagaattagaagtggagcctgaattgctgcagtctcatgatgaaacttgcacagatgaggacctgcttcttacagatgaggaaagaaagtgttttttttttttttcagatggaatctattcctggtgaagatgatataaatattgttgaaatgacaacacaagattaagaatattacataatcccagcacttagggaggctgaggtgggcagaccacctgaggtcaggagttcaagaccagcctggcctacatagggaaacctct

SEQ ID NO: 21 - DNA, 377 bp, Homo sapiens
  source 1..377: mol_type = other DNA
cagcttgagaggagacggggtctttcatatgtccagggtcctcctctcctgcctcctccgctagaggtggacctcaacccggggaaagggggtgtcctgaaggctgggattgtggcctcctcgaaggcgagggctctctggccctcaccctgcccacaagaccagccctgcatccccaattgccagttcctcttctatcctctccagaccctgcactacttcctcttttggggggattttctgttgctgtctccttcttggcgttcaaccctccatcttctctctctgtagttttctttgtctccccctttctccagggccctctcctacccacgcggcttggaagtgtgtccgaggctgtcactccaggtgacagc

SEQ ID NO: 22 - DNA, 613 bp, Homo sapiens
  source 1..613: mol_type = other DNA
caggtcagagtggagggctcccaccagggtttcctttagggtcctgaggggttggcatctgcccaaaccccctccagtctggctgaaatttcaaggtcaaggggtgccttctggcagtcaagggtgagcctgggaggggcagggcagggatttgcatccatctaagcaaagggcatcaagccaagtcatctgatgagagtgactccggttggggggtgggggcgtgtgggaggccgagcctgtcctcggatcagttgcgtactctgcccgccccctctgactcatgctgacaatcttcttccttcccctggccacctctctgcccacttgcttcctcagtaccttggtccagctcttcctgcaacggcccaggagctcagagctccacatctgaccttctagtcatgaccaggaccagggcagcactcctcctgttcacaggtgagcctggaccccaatgaagtagggctggggacccaggcccaagggagccagggccctgaactgggggctcaggctggggggttaggatctgggtaggaagagagactcagtcaagcctgagggggaggcaggcacatagggtttgagatttggagtttgtggagggaga

SEQ ID NO: 23 - DNA, 770 bp, Homo sapiens
  source 1..770: mol_type = other DNA
tctgattcctgcccttggaaaggtctaccaggaaatcatgagtgacttaagtaggacaaacaacacagaaggcataccgagaggccatacagttgaggctatgaagaaccattagaagctgaagttatgagtaagctgaagtcaggaggaagcaggaaccttgcctacagtgaagtatgaggttggaggttgtgggttgacgggggtgaggaaggctgttgatgggaagtgaggggacacggagaagcagaaacaccgaaatcagctgagttatacgaatgacaaggcacatgtgaagctgcaccaactctggctgctggtctcagaattgccctgaatccagcacagtccctgctaggccagctcatccctcatccctgttgctgggctcccacgtgttcttgtctcctttacccttgcatatggctttactatacacttcctttctctgaactcaacagagaaagcagaagctcttttcatactgtcaccagtgccaaaccgacccaaggctcgtaagacctgtttcctccacttctgggaagttccctaaaggacaggggtctagagacagggaaatgagaatgctagactgtatgttgctttggaatacattgctctgcttaatgaattagtacactttcatggaggtctgcctcctaggggaagggcagagaggagaaacagaaggcaacagatgatgaggatcaattgacactttactcatgctgatcctttcctctcacccaaatcccacccacccttcatgt

SEQ ID NO: 24 - DNA, 409 bp, Homo sapiens
  source 1..409: mol_type = other DNA
tttcctggaggcagggactttaggaagaagccattagcctagagacatatttcatacttatgagcacactcacagctaccaacccccacttgggcagttctctgttcctggaaggcaaagccctcagccacttcctggaagcagagaaggtgggggcaaagttgcaaggcctggtctccctggttcccgggttgggggaggcatcttggtgagtcagggccggacttgcagcagcccggaggctgggagaataacacagaaacacggagggtgctggggcccagttcttcaggtaggctgtcactgttgcccacccttcccctacagccatccccacagcctcctgcacccagagacagctggggtggaggagaggctggggcctgaatcccacatttgcccccactta

SEQ ID NO: 25 - DNA, 681 bp, Homo sapiens
  source 1..681: mol_type = other DNA
tggcttggttgagtggcaaagagggatgggatctggggaaagaatttggggagcccctgccagatcagctgctggatgaaccacccccagggtggcagccccagcctgtaagagacaagggacctggaacaaactgagcccatcatcccccgccccccatttcctgtctaccctccttccttccctgctggcagagctgaagtcagcacagtgtggtttcctaaccgtgcaaccggctggagcaagtggttcagctttctgaacctcggtttccctggcaagaaaatggaacaatcattgcacccacattatgaggttgagtcctaaatgagataatgcagacaaactgtttgacaaggcactaggtatgcagtaagtgctcagtaaaggccagctgcttgcccccggccatccctcttagtcctaggccccagctctgctgtgttgtgttctctgcaatgagagggctgcgctgtactcggttaccctcccaaggaacagcgccttccctcagcagccaggagccacagagggtgactaatgggggacttccaggccccagggcaggtcagcagggctgaggttttggggcgtgggattccccccagagagggagtggccaactccatcagtaggggttcctgcgaccgcacgcgttggcagggttgggttccaatgagc

SEQ ID NO: 26 - DNA, 418 bp, Homo sapiens
  source 1..418: mol_type = other DNA
ttgaaggctgggatttgaacccaagtcttctgggaggtgagtcagacgccttgcgctcttcccccactgtgcctggagaacgttgctacctagggtccatgctgacaagtttatatagatgggaacataacagagagaaggcggaagttccaggtcacccagtcattagcccagctgggtcccccttctccttccacccagaaatagccctggcgcctgccccagaccagccctagcctctgggctccccgacgcttgtcaccagttacatctccagccacactggcccagacaacatctaagccctggttgtggtcgccgcaccccagaggaagacagggaacagctgaggcatgacccctcacctcactgggtgcacagcccccgcagaggcatgcatggattggagggccttttt

SEQ ID NO: 27 - DNA, 437 bp, Homo sapiens
  source 1..437: mol_type = other DNA
cttcctcaattaaagaccaaagacaatgataagacaactcaacttaaatcttgctaattgtgaaactaacgttcccttttctgggtagcaacatctcataatacagtcctcaacagcagactgatatatatgacattgccagatatccaaaaaaagtccacgggaacttacaaatgtaatccttcacggctttctcaaagaggtcatacacgtgctctctgtccaggccatcctgggccatgctgatctcgtcatgcagccactccagccagagctctacgagacaaaaatacttcccgcattaataccaaagggtaggctaagcgcagggactttaccaagagacatcagcatgcaaacgtttcatccatcttacacaacctggcgttgtttccagaaattcacttcttaaaacctgaactaagcacataggaggt

SEQ ID NO: 28 - DNA, 447 bp, Mus musculus
  source 1..447: mol_type = other DNA
tgctagtaagttggcccatccatgcggggctcagagacacagagcaggaccccacaccagcctgacacactacagtgaataaaaggagcccggcacacaaatggctgtacatatctgggtctagttctgctaggttctggtgccaagtccgctgagcccctacccaagctgctttcctggtcaccgtgcagtgactcacggtcaggccactggggctaaccacagtacaacagtcccagacaaaggcatcttgcggcatctgtcctctctgagggaagtggtgggcagtgggggaggcatgcagttcctctttggtttggaacatatttcattacttagaggccagcagtgaactgcctggggtggctagacctttctttctttctttctttttgttttgcattgtagcacaatgttaaatacaaaccagctttctacctcagtgtc

SEQ ID NO: 29 - DNA, 632 bp, Mus musculus
  source 1..632: mol_type = other DNA
aaacaggcagtagctgagcattttcagctaaggatggagaagcccaagtctcctgggagacctcggcccagaacgtcagcaccactcacaagggcccaggcaccttgcaaaggcgggctccttgtcccttgggcccccagccactccccatggtttttcgaatcagacttaccgtgcccaacagagcatggggcagctgcctcagggcccagaagcaaacgactctgcctcagtgacagggccccagtccacactgccagacacttgtggtcccaatctgcccgccaacccccaacggcagggtgtggaagaaggaagcgagaatgaggttactcaggctggtcacgtgacgcgcccacacccccttgtttgtgacagctttggtgacaagtctgattaaagctgggtgggcagtgtgatggcggtttgttgggactgataacaatcttgaaagttcaaggaaggtatggggggtgcttactgtgaggggacctctacatgagacctcagagaagagtcacagccttgttctatgggcaactggagagaggcaaccagagagaacccctgccatggaggcaaaggccagacagaagggagggagggtctcaatggagatggagaatgggagg

SEQ ID NO: 30 - DNA, 640 bp, Mus musculus
  source 1..640: mol_type = other DNA
acacaagcctgtaatcccagctcagacaaggaagggacaggaggcttggggtgtgatggtctccctcagctacttagcaattgaaagaccaccccagactgcatgagaccctgtcttagaacagtgtggaccttcacggggcccagtcctggatgctcacaacgccagccagctctaacagagccacctgctccacaggatccaggcgttctggccgttgagctcctgggggccagggagacccacacctgcttgctctgggggaaaatggctcgaaggaagttggttaagaactagccttggagaaaggggaagtgagactgggcacatgctcctggtatttcagggctctgaactacggtcagacagtaggaagaccctttctctgaccttgaggcctcttgtctgagggagaccggctttccttaatgcataagcatgcctctgcctcttcatccatcagaggtctcagacccccaggcatcctcaggcagatgctgcccagcagcctaggcgatgcctgccttcagtgggcagtgagcattcttgttcctgcttacacagccaggaagcccgggttgtgtccccgactagagatcccttggtacacagtggcagccctgcccaggaattgtgttga

SEQ ID NO: 31 - DNA, 488 bp, Mus musculus
  source 1..488: mol_type = other DNA
tcaaaaacaccgacatcgccaaggagctctgcctgccgcctgtgaaactgcactgctccagtgagtctgccccacctgccaggcagcttggatctgttcagagtctgctgttcccaccccacggtggccgggcagctgagacaggcagctcttcccccaccctgcgcttccccagtctgcctttgctgtctctgagtgtgtctgcagtgcctgaggccacagtgcactacctaccacactgccccacgagagccttcccacactgctaagcctcttccagaaagcagaagtaagacgtcaccctgacatgctcagctttctgcccccttccagctctcggcttctgcttcgcccatagcctcaccctcggtatcctctcagtactagccttccagtgtggggctgcagcaggaggcatgcagactgctgccaggtgacaagcactctctgcaataccctcactgttccagccagaaaggtgtgcaagt

SEQ ID NO: 32 - DNA, 626 bp, Mus musculus
  source 1..626: mol_type = other DNA
aaatcagagatgcagctgaggcccagaaagggtaactgacttgatccagagaacccagccacacaaggggcagaggccagcccttcctcccagggttccctgtccatgaccagggcctacccaaggacccacctccatcatagatctctggaaaacctcagggtggagagctgggctccagagccctggactctcacccacatgctgcatctagggtccaatccaggcttcgtccctcactggccccctagagtaggccactcagcagtttccctaccataagaggaagatgggtacccgtgcaaggtgctaggctgccacacactctgccacatgggtgcacccctggaggttagaggcctgagggcaactgcaccatgtgagcagacagacaggaagagcctggtgagaagccagctccggtcagagcctttgaagacgctctggcaaaccacaagagacaaacagcgaagttaccacaggctgcccaggatcctgaggaagtctgagctcactggctttgggttccttccctcaaagcactcacccattcagccattcacccattcagccattcacccattcattcattcattcagccattcagccatctgtttattcattca

SEQ ID NO: 33 - DNA, 531 bp, Homo sapiens
  source 1..531: mol_type = other DNA
tgccagttgaacaagatgctgtctcagagatgaacaagccatgatagaatatttcttctatcctcagagtttagttgagaaaattgaggcccagaaagggccagaacggtcctggcacacagtaggtgaccaacaagctggtcacttcctccctttctgctctccctccctccccttctgctctccctccctcccctcctgatcttgccagtcactagagagagaactctgtgtcacccatggatagaagttccacaaacaccgacaagggaaaactaaggggcaatttcattgaaacaacaagttattttgaaaactattggtccctgttacagttgattgtttggaaaccaagagatgtgtgtcccacacagaaaatttctgggaaatgttaccagtctttaaaaacatttggtaacagtaactgttttcaaaataattggctgtactacaggcagagggggaaaagaagtcaggagaagtgcactgtatatagaaaattggtaataaggggcatgaagtttcagagct

SEQ ID NO: 34 - DNA, 598 bp, Homo sapiens
  source 1..598: mol_type = other DNA
gtgggcagatcacgaggtcaggagttcgagaccagcctgcccaacatggtgaaaccctgttgctactacaaatacaaatacaaaaattagccgagtgtggtggtgtgcacctgtaatcccagctataaaggaggctgaggcagaagaattgcttgagcctgggaggctgggagccaggctcctgggagcctggaggttgcggtgagctgagatcaagtcactgcactccagcctgggcaaaagagagagactctgtctcaaaaaagaaaaaaaaaaggtgtccaagaaacctgggcaaagagcctgaactcctcagtggaggaggctcctagtgcaaggagtggaaaatgagggacagggaggggaagcattggtagaaggtgtacggaaccttcttggagttgagctttgatcttgaggaaaactggcagctgggttccaccaaacaacacactccagctattgtttccttgaactttggcctacattaatgaagaatctggggcaaatttttcacaggttcttccacactttatttttaaattttttgataactttattgagatataattcacataccgtacatacaattcaccca

SEQ ID NO: 35 - DNA, 781 bp, Mus musculus
  source 1..781: mol_type = other DNA
tcctgcgtccgcacgcgtagcctggtcctggaatagcacccagatatactggggaggttcatgaaagagaaaaggtctaagctgagagtggtcattagcctgagagatctgacctttcacagaaaagatttagtgtaattcggtaccgcgtgagtgtgcttataaaatgccaattcacctccactctcagaagacttttctaacatgcacctcatttcttactatgtatctggcctgaaataggagcattggctaaaggtgaggattcccaggggttgctactgtaattaaaacagaaagccagttcttggagacatggacactcagaacttgatggttaatgattatggttaatgccatctggttatgagcagtagaattctagggtgggggatactaagagtctgctgtaatatacatactactcaaatgtaagatgatatgcttatatttatgatcctttaacatcccaatctctggacctcagaagagaagactggctctgggagctaattaacatgcagtcaatatcccaagtctctctttgttcaagaaggcacaggctgaactcattaaactgccagcagaccagaaaaataagttatttgaaagccaacacaggaaaacttatttataaagctcagcagattttaaaagatcaaaacaaaacccattaataataggctgggcttgtagctaagtagagtgattgcgtgaagtcttgggttcaggttcgattcccagcgcagggtcaactttgtaaggcagtacacgcttgcaa

SEQ ID NO: 36 - DNA, 705 bp, Mus musculus
  source 1..705: mol_type = other DNA
tgcccagaagctatgtgaccacatgtaagtctcccattgcatggagatagaaaatttagagctagtagccctgcagaatctagttgcagaccccttcatgggccccgaatttcctgtcttgtcatcacagcaaagcgaggactgggaatggtctcagcatagtgctggctcagagccggcaaagttcctgctgttcttcaagggaaagttgtttgggttctttgtccagtaacttggtaacaagaaaactctcacgctggtcaaaaaaaaaaaaaaaaggaaaagcctcgggacccccatgccactcccagcttcctctgctccccccttgaaagcagagactagagcagtgggtggtattgggcctgttctgcccagaggctgaggagggtggcccgcccccaactgccagccagtacaagacaaaagacccttgacagttccctggggcggttcagctgtggcccggctggacaaagcaagggtacagggctcggtacagctctgcaaagtccttggggcttgtcttggcggagacttgggcttttgagaagctctgtccagggaagctctataaacccagacaacaaaatgacccctctcctcttacttgaagccagaagtcaaatggaagcaacaggccctggcctcaccctctttcaccccaaggccacatctctgaactagaggtggcagaagcacctt

SEQ ID NO: 37 - DNA, 581 bp, Homo sapiens
  source 1..581: mol_type = other DNA
cccctcacacacccacacatgtatatacactcacatcctgaagcacactcagacacatactaactctcagaaacacacttatccctcagagggggtaggaccagcctaggaaagagtccttctgggagaatggcatggctggtgactgccaaggggtaggcagaagcctaggactgggcagggagcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctctcttcttgcagggagggggtgggaaaccttccccataaccctggcatttcacccccagcccacacaggtggcttttggagaagctcaagtgccagaatttgacccgaaaactccaggttccacaacgccccctgcttccttcccaggccaggaagccttggaagtccagcccccaccaacctgtccccta

SEQ ID NO: 38 - DNA, 303 bp, Homo sapiens
  source 1..303: mol_type = other DNA
tgaggagaggaggccgagaaactgggctgagcaggacaaaacctcccctgtgaaccctgccaaggggaggggccccttattaaaacttactggggcatagccaggcccccctggagggagggggtctgaggggcagggaagggggctgggtggaggcaactaggactctggcctccagccagtcctcccctgggtggctgtttctgtggctgggctgtggttcacaccatctgtagcccttcagttgtccccttctccataccaaagtgctgtttcgcctagtcctgccctggtcccattcat

SEQ ID NO: 39 - DNA, 184 bp, Mus musculus
  source 1..184: mol_type = other DNA
ataaggagggtctttgtttctcttccctgggaaccgagcagcccctttctctgagggtagagttggggcatggctcaacagaacgacagtcatcctggctagtaagactcagaggctggccttcaaaggcttgagctcctgggtgactgagcagtggagaaacagaatcctgcccttgaattgc

SEQ ID NO: 40 - DNA, 552 bp, Synthetic construct
  source 1..552: mol_type = other DNA
ataaggagggtctttgtttctcttccctgggaaccgagcagcccctttctctgagggtagagttggggcatggctcaacagaacgacagtcatcctggctagtaagactcagaggctggccttcaaaggcttgagctcctgggtgactgagcagtggagaaacagaatcctgcccttgaattgcataaggagggtctttgtttctcttccctgggaaccgagcagcccctttctctgagggtagagttggggcatggctcaacagaacgacagtcatcctggctagtaagactcagaggctggccttcaaaggcttgagctcctgggtgactgagcagtggagaaacagaatcctgcccttgaattgcataaggagggtctttgtttctcttccctgggaaccgagcagcccctttctctgagggtagagttggggcatggctcaacagaacgacagtcatcctggctagtaagactcagaggctggccttcaaaggcttgagctcctgggtgactgagcagtggagaaacagaatcctgcccttgaattgc

SEQ ID NO: 41 - DNA, 606 bp, Homo sapiens
  source 1..606: mol_type = other DNA
actgagggataattctgatgtgtctattccagcagaacatttgaaagacgtgtccaattcaggccagagcgttgctctcagagcgaagggtatcaccatgctctagagttcagtctcctctgcacatacataaacacaagtgcatagcgccccaacgctgtttgtcccctcggcctcacagctctgccagttggcaaacccaagccaggaagttgtcaattccaattgcgtttaggccatgtagtcctattaaagaaggcctgctggttaaggggcgtcctctgtttccaaagcatgggaaaaaaaaaaaaaaggctatgcttagattggagctggcaactttcactgactagagttttcaaggagctatgaatgagtttaacaaaggaggtctgtggctagtttctggctgaagtccatgtcaacagtttttaaggtcacaggccacactaactaacatttattcccagcagaccctgttctaaacacaagctgtaagtagtacaaatttggaatttttctgtaatcccttgagatattcctgatccttccacatgtttctgctcaattaaaaacaattctcactttgcaccacaagagaggcca

SEQ ID NO: 42 - DNA, 361 bp, Homo sapiens
  source 1..361: mol_type = other DNA
tgccaaagctggacaggctaagccagaggcaaggcaggggtgccagccctttgcccagggccagagggaggccaggaggaggtgcctgggctggttggcaccttctccctatccacgctctccctccagtcccaagtgacctttcccccagatcccagggtccaggcccgccctcggctggcaggtgtgggcacagaggcagctgggattggtcgcagctggcggaggcgcgtcccaggctccggcagaccgctggaacagctgagcagagcaggtggactgctgagatagaccagggacaccaggcagccacaggcctgtcagaccaggacccttaccctctagacatggcctcggtccc

SEQ ID NO: 43 - DNA, 607 bp, Mus musculus
  source 1..607: mol_type = other DNA
tgtaggctgttcagtctgcagggttgggtggagtactccatctctttcctctctccccatcttcctagctcccagccctctcacattccaacctccattttcttgtcctctaccccattatttttctaactccttccccctccttttccttctctctagtttctccccctaccttttgctgacccattcccttcccccccattttctctgccaccttttacttaccccccccccccatcctcctccttttcttctgcttcgaactaattgcttaagaggtgaagaccaggtcggcttgtgaaaaccctgatccatcaccatggtgatgctaattcaagccgagttcaaaggaaatgactccagttaaatctaacaagctggagccaggcgcccagcaaggaccagtgccaggaggagggaggcccctcgcatgccagggcccagaaaaataggaaaaacaaccacggacgctcacacctctactctggtcctctcttggcatccttcagatctgcccaaacctaaagccgaccagtgagccccctgccttcagaccagactctacctaagtcatctcaaatagttggacatccagcaacatccccat

SEQ ID NO: 44 - DNA, 306 bp, Mus musculus
  source 1..306: mol_type = other DNA
ttgaaggctggaattagggggtgaggacccgcgtccttcgagccttgagggacatatgcaaagaattgctaattgcaactaaacgaggcctcggcgtgggggaaggggcagcccgggctgtcaggggctaccgcctctttgagtgcccgggtaaggccaggccagcccgctcttctcatgtaaagcagctttttgtctgggggagccagagctggggccaggggctggggaaggggcccaagcctttggcaagtttcttgttccccagaaagggagaagggtgccgaggcctcctcggagtgagga

SEQ ID NO: 45-46 - skipped (2 entries)

SEQ ID NO: 47 - DNA, 245 bp, Homo sapiens
  source 1..245: mol_type = other DNA
ctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagtt

SEQ ID NO: 48 - DNA, 735 bp, Synthetic construct
  source 1..735: mol_type = other DNA
ctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagttctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagttctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagtt

SEQ ID NO: 49-50 - skipped (2 entries)

SEQ ID NO: 51 - DNA, 209 bp, Homo sapiens
  source 1..209: mol_type = other DNA
agcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctct

SEQ ID NO: 52 - DNA, 627 bp, Synthetic construct
  source 1..627: mol_type = other DNA
agcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctctagcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctctagcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctct

SEQ ID NO: 53 - DNA, 120 bp, Mus musculus
  source 1..120: mol_type = other DNA
aagcaaagcccaggcatcctatgggaggtctggctaaaagccagggtcctacttgcccccacgctccacccaactcaatccctgccccttagacctcctcccaccagggggctggctaca

SEQ ID NO: 54 - DNA, 360 bp, Synthetic construct
  source 1..360: mol_type = other DNA
aagcaaagcccaggcatcctatgggaggtctggctaaaagccagggtcctacttgcccccacgctccacccaactcaatccctgccccttagacctcctcccaccagggggctggctacaaagcaaagcccaggcatcctatgggaggtctggctaaaagccagggtcctacttgcccccacgctccacccaactcaatccctgccccttagacctcctcccaccagggggctggctacaaagcaaagcccaggcatcctatgggaggtctggctaaaagccagggtcctacttgcccccacgctccacccaactcaatccctgccccttagacctcctcccaccagggggctggctaca

SEQ ID NO: 55 - DNA, 493 bp, Mus musculus
  source 1..493: mol_type = other DNA
atgtgcatacctgcctctgagaaggaccaaagtcagatgcactctcaatgtaagggcttgggggtggaacacctcaagttccacacacaccccaatctcctacaggagtttcctttagaggacctgtctcctccaaggggctctgttagaccaagtgggaaagtctctaggaactgacttcagaggtttggggctgtgccagtcccgcggcgcactctgccacggaggagacctggagctcctgggttggccaccttgctccctgcctcctcccacctcctccccagcaggttatcagcatgaaaacaggaaggctggcagtgctattaataaaacatgaagcagagatctgccccatcaggcagcggaggcaggcggcagctcggcagccccgagggagggagagaacccccgcatctgggcctgggcaggggtattctcttctcacagatgccacaccccaggagccacctccattttgcgggactgctgt

SEQ ID NO: 56 - DNA, 53 bp, Synthetic construct
  source 1..53: mol_type = other DNA
gggctgggcataaaagtcagggcagagccatctattgcttacatttgcttctg

SEQ ID NO: 57 - DNA, 68 bp, Synthetic construct
  source 1..68: mol_type = other DNA
gaggtaggcgtgtacggtgggaggcctatataagcagagctcgtttagtgaaccgtcagatcgcctgg

SEQ ID NO: 58 - DNA, 68 bp, Synthetic construct
  source 1..68: mol_type = other DNA
gaggtaggcgtgtacggtgggaggcctatataagcagagctggtttagtgaaccgtcagatcgcctgg

SEQ ID NO: 59 - DNA, 120 bp, Synthetic construct
  source 1..120: mol_type = other DNA
gattcagccgggagcttagggaggggaggtcacttcataagggcctggggggggagttggagccacgagtcgtccagccggagccccgtgtggctgagctccggcctcagaagcatcccc

SEQ ID NO: 60 - DNA, 120 bp, Synthetic construct
  source 1..120: mol_type = other DNA
gattcagccgggagcttagggaggggaggtcacttcataagggcttggggggggagttggagccacgagtcgtccagccggagccccgtgtggctgtgctccggcctcagaagcatcccc

SEQ ID NO: 61 - DNA, 867 bp, Synthetic construct
  source 1..867: mol_type = other DNA
caggaacatccaaactgagcagccggggtcccccccaccccccaccccgccccacgcggcaactttgagcctgtgctgggacagagcctctagttcctaaattagtccatgaggtcagaggcagcactgccattgtaacgcgattggagaggatcacgtcaccggacacgcccccaggcatctccctgggtctcctaaacttggcggggagaagttttagcccttaagttttagcctttaacccccatattcagaactgtgcgagttggcgaaaccccacaaatcacaacaaactgtacacaacaccgagctagaggtgatctttcttgtccattccacacaggccttagtaatgcgtcgccatagcaacagtgtcactagtagcaccagcacttccccacaccctccccctcaggaatccgtactctccagtgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcaggtcaccagacgctgacagctactcagaaccaaatctggttccatccagagacaagcgaagacaagagaagcagagcgagcggcgcgttcccgatcctcggccaggaccagccttccccagagcatccctgccgcggagcgcaaccttcccaggagcatccctgccgcggagcgcaactttccccggagcatccacgccgcggagcgcagccttccagaagcagagcgcggcgcc

SEQ ID NO: 62 - DNA, 720 bp, Synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 63 - DNA, 720 bp, Synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 64 - DNA, 1299 bp, Synthetic construct
  source 1..1299: mol_type = other DNA
atggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 65 - DNA, 1056 bp, Synthetic construct
  source 1..1056: mol_type = other DNA
atggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaa

SEQ ID NO: 66 - DNA, 50 bp, Synthetic construct
  source 1..50: mol_type = other DNA
gaagctacccctaacacactattctacacacagaaaatgctcttcactag

SEQ ID NO: 67 - DNA, 150 bp, Synthetic construct
  source 1..150: mol_type = other DNA
gaagctacccctaacacactattctacacacagaaaatgctcttcactaggaagctacccctaacacactattctacacacagaaaatgctcttcactaggaagctacccctaacacactattctacacacagaaaatgctcttcactag

SEQ ID NO: 68 - DNA, 66 bp, Synthetic construct
  source 1..66: mol_type = other DNA
gactacaaagaccatgacggagattataaagatcatgacatcgattacaaggatgacgatgacaag

SEQ ID NO: 69 - DNA, 30 bp, Synthetic construct
  source 1..30: mol_type = other DNA
tccggactcagatctggaggctccggaggc

SEQ ID NO: 70 - DNA, 378 bp, Synthetic construct
  source 1..378: mol_type = other DNA
ccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggagaggcttcccgcctggcgcattacaacaagcgctcgaccatcacctcccgggagatccagacggccgtgcgcctgctgctgcctggggagttggccaagcacgccgtgtccgagggtactaaggccatcaccaagtacaccagcgctaagtaa

SEQ ID NO: 71 - DNA, 246 bp, Synthetic construct
  source 1..246: mol_type = other DNA
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgg

SEQ ID NO: 72 - DNA, 609 bp, Synthetic construct
  source 1..609: mol_type = other DNA
gcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 73 - DNA, 204 bp, Synthetic construct
  source 1..204: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 74 - DNA, 479 bp, Synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 75 - DNA, 78 bp, Synthetic construct
  source 1..78: mol_type = other DNA
ggcagcggcgccaccaacttcagcctgctgaagcaggccggcgacgtggaggagaaccccggccccggagctagcgga

SEQ ID NO: 76 - protein, 21 aa, Synthetic construct
GSGEGRGSLLTCGDVEENPGP

SEQ ID NO: 77 - protein, 24 aa, Synthetic construct
GSGQCTNYALLKLAGDVESNPGPP

SEQ ID NO: 78 - protein, 25 aa, Synthetic construct
GSGVKQTLNFDLLKLAGDVESNPGP

SEQ ID NO: 79 - DNA, 130 bp, Synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 80 - DNA, 141 bp, Synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 81 - DNA, 146 bp, Synthetic construct
  source 1..146: mol_type = other DNA
catgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 82 - DNA, 149 bp, Synthetic construct
  source 1..149: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctg

SEQ ID NO: 83 - protein, 743 aa, Synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 84 - protein, 736 aa, Synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 85 - DNA, 747 bp, Synthetic construct
  source 1..747: mol_type = other DNA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 86 - protein, 189 aa, Synthetic construct
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 87 - protein, 11 aa, Synthetic construct
RPKPQQFFGLM

SEQ ID NO: 88 - protein, 9 aa, Synthetic construct
CYIQNCPLG

SEQ ID NO: 89 - DNA, 1353 bp, Synthetic construct
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaagggagctacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 90 - DNA, 1353 bp, Synthetic construct
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 91 - DNA, 1353 bp, Synthetic construct
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 92 - DNA, 1863 bp, Synthetic construct
  source 1..1863: mol_type = other DNA
gcggccgcacgcgtacatttcccttgccccttgcttttatgcttttactgttatttcttagccctgaactgagttgcgagactcccaggtcctagtctcagatctgccacttttcctatttatacaaggaaaagcaaaatcccagtaaaggacaaggaaaaatcaaacatgaagcctcccggccttctctgagagccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagtcatgtgttctgaggaaccaagaagcgagcttactcggtccatcagtggtttaattgttgggcctttctctgcaggtcttattttttagatgaaaagtcaggatacatggtaaaggtcagtgcttggggagattatggtgtcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 93 - DNA, 1609 bp, Synthetic construct
  source 1..1609: mol_type = other DNA
gcggccgcacgcgtgcacccgagatagaaggacccattgttttctaaaactagttaccactcttccctttatcctttcttttgtaccttttagcgtttgcttaatcagaatattttaatccctaatgtggtgactcggtgtaatttatctcattaagcctctcctattttctcctgtttacatgactaatattattgtgttttatagagcttataccaacaaatcactcctttgtcccattgtttcagctccaggccaaaaaccagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 94 - DNA, 2305 bp, Synthetic construct
  source 1..2305: mol_type = other DNA
gcggccgcacgcgtgtccagatgctgttgccgtggtaatactaaatcgtgtccaaagaggaggctgagattcctttttagttaaccttgtccacatgatttgactatccagcaggcttggattcccaggccccagagacttcgggactgttttccagcacttgaatgtggtatataagtgctgatatcaaaagggatatgtaggaaagacaagattataggcagggcattgtctgaccaggacgaggcccgaagcaaaagtctttgtttgtctctggcacctcttgataattgttgttttactcctagaacccacccactgatccccatccatccctagtaccagtcacggctgctctagcatttcggccttgtcaactactgcaggccctgatcaaggaggcctctgttcatggggtaaggttaaggggtgggttctgggaggagggtaagactgtagtgcagattcgagacctagggaggtggggccagtttctcactcaatgagcctgcctgttgtttagctctattgtatagggttcttctatctctccctttgtcagctggattcaagggacaggctgggaattgtgcagggtacaattggtctctttctaccattcctttcgatgaaagcccttcctctccttcctttgatgagttcccagattcacactgaatttccatcatggagctggtccttgaggcgggcccagggcatgggaaaggagggagggagggttgggagctcttggtgcctgcttaaagagacagcatcaggatcaacacagtcccttgatgaacctggatctgtgtaagtcacattggttctgttgatctctgtcagaccgctgtgaagagcgctggtgagaagggcttaaagttggagtatcaactatcacagattgattagacttgttttgggagtttgtatacaaatgtagccaaccatgacatgatctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 95 - DNA, 2022 bp, Synthetic construct
  source 1..2022: mol_type = other DNA
gcggccgcacgcgtctggccttaggtctctggggccccagcagccccatgtctgcctccttaaatcggccttgaggttaagtgtgtctctgtctgtctgtctgtctctctgtctctctccagctctgctgaggaagtactaggctctcctcagggcctcgccgcccacagtaacaggtcactcagagcccacagtaacaggtcactcagagcccacagtaacaggtcactcagagtggcactgctttgctcttgctgtttgtaagagaaaaacacgctaagcttcggagcctggcaccaccaggtttggccactcttgattatttcccttgttcccacccctcttctcttgccctcaagtcctgaacccccttcttatcaccctcccccagcccagctgctgcatgtgccaacccctacccagcagaaaaggaaggtcttggaaggggatcaatggaggttctgtgtgggcttgcagctgaaatggccctgcacacagccacagatgggcctctgctccccctccacatttctgcatcaacaaagcgtgaccttgtttagatgacagtcgccccatgtctcccccatgacaatgtttgccttgcttttgcctcctagtgggctgcctcaggacatctctgttgcctgagactggaaaagggtgatgggatgggaagcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 96 - DNA, 1662 bp, Synthetic construct
  source 1..1662: mol_type = other DNA
gcggccgcacgcgtcaccactctgtgcttagaaccaagagctaggagagcaagggagcctgggactggcacttttcagctagggggagctggtggtggcagggcagagaggcccattgcgcagctggtaacaggctgtgcctggccaggcctcccagctgcccctctgtctcaggcccttattgcacacaacatccagcccagccccctacgcagctgccttcctcatgacacagccctgtgttgtcctgttggccactgaccactgtgcctggggaagggccaggccttcctgggaggaggaactcgagagcctgtggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 97 - DNA, 1794 bp, Synthetic construct
  source 1..1794: mol_type = other DNA
gcggccgcacgcgtgagggtctggccgtgagtgtgggagagtccacactgggcgaggagggagggcggggctttgaacatcagacctcagggtgggtggcctgagctgccagtggggccaccagtagccacaagcaaatatctccaccaacaaggcccgtctgtctcgggacaagcatgtctttgtggcgcttgctccccttctgtcctcccccacagctggccctgtccgcccgtgcccagtcaggccccgcagaacccaagaggggcttcccatgccatttaggaagggctgtctctttaaaaaaaaaaaaaattacaaaacggttgatttcccaagaggttgacacagcagcacgtacagtaagatgttcctcacacctgcggggatgccatggtcccagcccggggcacccgttacccaggaaggtggtgagagtggtgctgggaagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 98 - DNA, 1752 bp, Synthetic construct
  source 1..1752: mol_type = other DNA
gcggccgcacgcgtgcgagtccaggggctctaccccctcttctgccctccttcccctccctccagggtggttcagtactcgctgggatgggggactctaacccaagtcctccccgcacccatggttctagtcacatctgtcaaagggatccaggcaagaacaaaggcctagtgtgcctcggacaaaggagaaaggtcccgccagccctcacccctccctgggccctgcgtctgccccctcgggaccgccaccatggaaaccccctcccacactcactaatagcccccgtgggtgcccaggacctgagccatcgccagggcaacagggctggttccgacacaggcgtgtgtcccgcagtgtccattcccaagtgccctcccttcgatggggaacacggcaggactccatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 99 - DNA, 2258 bp, Synthetic construct
  source 1..2258: mol_type = other DNA
gcggccgcacgcgttcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaactcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaactcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaacgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 100 - DNA, 1832 bp, Synthetic construct
  source 1..1832: mol_type = other DNA
gcggccgcacgcgtaatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgtaaatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgtaaatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgtagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 101 - DNA, 1709 bp, Synthetic construct
  source 1..1709: mol_type = other DNA
gcggccgcacgcgtgtaacaggctgtgcctggccaggcctcccagctgcccctctgtctcaggcccttattgcacacaacatccagcccagccccctacgcagctgccttcctcatgacacagccctgtgttggtaacaggctgtgcctggccaggcctcccagctgcccctctgtctcaggcccttattgcacacaacatccagcccagccccctacgcagctgccttcctcatgacacagccctgtgttggtaacaggctgtgcctggccaggcctcccagctgcccctctgtctcaggcccttattgcacacaacatccagcccagccccctacgcagctgccttcctcatgacacagccctgtgttggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 102 - DNA, 1904 bp, Synthetic construct
  source 1..1904: mol_type = other DNA
gcggccgcacgcgtagccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagtagccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagtagccttacaaggttctgttttgtcttttcagctgtgatcttagctcagagaaggtcatgttttgtttttgtcttttgtcttcctgcccgttccagttggtcctagtcttttgagatggggattgctgggaggtcacaatgcagggattgtttagatgacagggctgggagagcagcgctgaagtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 103 - DNA, 1652 bp, Synthetic construct
  source 1..1652: mol_type = other DNA
gcggccgcacgcgtcagaatattttaatccctaatgtggtgactcggtgtaatttatctcattaagcctctcctattttctcctgtttacatgactaatattattgtgttttatcagaatattttaatccctaatgtggtgactcggtgtaatttatctcattaagcctctcctattttctcctgtttacatgactaatattattgtgttttatcagaatattttaatccctaatgtggtgactcggtgtaatttatctcattaagcctctcctattttctcctgtttacatgactaatattattgtgttttatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 104 - DNA, 2006 bp, Synthetic construct
  source 1..2006: mol_type = other DNA
gcggccgcacgcgtagcaagttttctacctgggatgcccccaagggtagcaggccatacttcctaagtgtgcatgttggtggggagggagggtatcaaggagtatgtccctggagaagggagaaaggagggggcgtgggaaggggcctgtggaacgcacgtgtctcatttcctctttcggaatgtccgctgtgggccccaacaaaggaacttcggttggaactggggtgaggaagcccccgaagatcaacttctcttcctttaacatttggctctgctgagggccctggggttggaacacccaccttcttagaaagaaactgaaaggccttaggagaaaccccgccacttccccttcctcggtaagaggaacttggcgttgccctgacaggaaagtcttggtggggcttctcggaagccgccaccgctgcactgctgagcaaaaccacaggtcaggggccaggccgggggccaggctagtgaggactggccattgataaagtcacaggctccatctaagcccatcggcagaactggggagctgccaacatttcctcttccagcaccgcctgccaactcctgtttgggagccaccaacccctcagctctgccaatgggtccctccccactcacaccagccctccaaccaggatcgttctgtgtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 105 - DNA, 1913 bp, Synthetic construct
  source 1..1913: mol_type = other DNA
gcggccgcacgcgtgggtgattctcccctgcctctcccgggatctttgccaagctcgtcctgttcagcaccaaagacagctcttgggtgccgccttcctggcccaccacccccttgggtgtgggtggatggtaccacctcactcaacatgcttgacgtggactaggcacacctgggtggagcccctcagcatgctgtgctctgcccaggcaataaccctggcaggagtgggcagcccttagacgggagttaggtcccagcaggcatcaagagggtgagagccactccttactgagtgaggggacccataccaactgccttggcctgggcttccttatgaggtctccagcacctcagctgatctgaaactgaggggcaaagaggaaacagaaactggccaggggccctagaacagaaatgcagaacctgaaaccaaatgtagaacagaaagcctgagaaccagctacgcccatgagctgcagacccatgggctgagaaaccagggactggggtgccagggaggggtgggagagcctgggagtagccacacagcactaggtcccaatgcttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 106 - DNA, 1836 bp, Synthetic construct
  source 1..1836: mol_type = other DNA
gcggccgcacgcgttctgatggatctgggcaaagtcaattgaaaaccttctggaaaaaaattcaccattttagatgccattaagaacattcatgattcatcagagggggtcaaaatatcaacattaacacgagttcgggagaagctgattctgattctcatggatgactttgagtggctcaaggcttcaggggaggaagtgactgcggatgtgtggaaatagagaaccagaattagaagtggagcctgaattgctgcagtctcatgatgaaacttgcacagatgaggacctgcttcttacagatgaggaaagaaagtgttttttttttttttcagatggaatctattcctggtgaagatgatataaatattgttgaaatgacaacacaagattaagaatattacataatcccagcacttagggaggctgaggtgggcagaccacctgaggtcaggagttcaagaccagcctggcctacatagggaaacctctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 107 - DNA, 1735 bp, Synthetic construct
  source 1..1735: mol_type = other DNA
gcggccgcacgcgtcagcttgagaggagacggggtctttcatatgtccagggtcctcctctcctgcctcctccgctagaggtggacctcaacccggggaaagggggtgtcctgaaggctgggattgtggcctcctcgaaggcgagggctctctggccctcaccctgcccacaagaccagccctgcatccccaattgccagttcctcttctatcctctccagaccctgcactacttcctcttttggggggattttctgttgctgtctccttcttggcgttcaaccctccatcttctctctctgtagttttctttgtctccccctttctccagggccctctcctacccacgcggcttggaagtgtgtccgaggctgtcactccaggtgacagcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 108 - DNA, 1971 bp, Synthetic construct
  source 1..1971: mol_type = other DNA
gcggccgcacgcgtcaggtcagagtggagggctcccaccagggtttcctttagggtcctgaggggttggcatctgcccaaaccccctccagtctggctgaaatttcaaggtcaaggggtgccttctggcagtcaagggtgagcctgggaggggcagggcagggatttgcatccatctaagcaaagggcatcaagccaagtcatctgatgagagtgactccggttggggggtgggggcgtgtgggaggccgagcctgtcctcggatcagttgcgtactctgcccgccccctctgactcatgctgacaatcttcttccttcccctggccacctctctgcccacttgcttcctcagtaccttggtccagctcttcctgcaacggcccaggagctcagagctccacatctgaccttctagtcatgaccaggaccagggcagcactcctcctgttcacaggtgagcctggaccccaatgaagtagggctggggacccaggcccaagggagccagggccctgaactgggggctcaggctggggggttaggatctgggtaggaagagagactcagtcaagcctgagggggaggcaggcacatagggtttgagatttggagtttgtggagggagagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 109 - DNA, 2128 bp, Synthetic construct
  source 1..2128: mol_type = other DNA
gcggccgcacgcgttctgattcctgcccttggaaaggtctaccaggaaatcatgagtgacttaagtaggacaaacaacacagaaggcataccgagaggccatacagttgaggctatgaagaaccattagaagctgaagttatgagtaagctgaagtcaggaggaagcaggaaccttgcctacagtgaagtatgaggttggaggttgtgggttgacgggggtgaggaaggctgttgatgggaagtgaggggacacggagaagcagaaacaccgaaatcagctgagttatacgaatgacaaggcacatgtgaagctgcaccaactctggctgctggtctcagaattgccctgaatccagcacagtccctgctaggccagctcatccctcatccctgttgctgggctcccacgtgttcttgtctcctttacccttgcatatggctttactatacacttcctttctctgaactcaacagagaaagcagaagctcttttcatactgtcaccagtgccaaaccgacccaaggctcgtaagacctgtttcctccacttctgggaagttccctaaaggacaggggtctagagacagggaaatgagaatgctagactgtatgttgctttggaatacattgctctgcttaatgaattagtacactttcatggaggtctgcctcctaggggaagggcagagaggagaaacagaaggcaacagatgatgaggatcaattgacactttactcatgctgatcctttcctctcacccaaatcccacccacccttcatgtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 110 - DNA, 1767 bp, Synthetic construct
  source 1..1767: mol_type = other DNA
gcggccgcacgcgttttcctggaggcagggactttaggaagaagccattagcctagagacatatttcatacttatgagcacactcacagctaccaacccccacttgggcagttctctgttcctggaaggcaaagccctcagccacttcctggaagcagagaaggtgggggcaaagttgcaaggcctggtctccctggttcccgggttgggggaggcatcttggtgagtcagggccggacttgcagcagcccggaggctgggagaataacacagaaacacggagggtgctggggcccagttcttcaggtaggctgtcactgttgcccacccttcccctacagccatccccacagcctcctgcacccagagacagctggggtggaggagaggctggggcctgaatcccacatttgcccccacttagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 111 - DNA, 2035 bp, Synthetic construct
  source 1..2035: mol_type = other DNA
gcggccgcacgcgttggcttggttgagtggcaaagagggatgggatctggggaaagaatttggggagcccctgccagatcagctgctggatgaaccacccccagggtggcagccccagcctgtaagagacaagggacctggaacaaactgagcccatcatcccccgccccccatttcctgtctaccctccttccttccctgctggcagagctgaagtcagcacagtgtggtttcctaaccgtgcaaccggctggagcaagtggttcagctttctgaacctcggtttccctggcaagaaaatggaacaatcattgcacccacattatgaggttgagtcctaaatgagataatgcagacaaactgtttgacaaggcactaggtatgcagtaagtgctcagtaaaggccagctgcttgcccccggccatccctcttagtcctaggccccagctctgctgtgttgtgttctctgcaatgagagggctgcgctgtactcggttaccctcccaaggaacagcgccttccctcagcagccaggagccacagagggtgactaatgggggacttccaggccccagggcaggtcagcagggctgaggttttggggcgtgggattccccccagagagggagtggccaactccatcagtaggggttcctgcgaccgcacgcgttggcagggttgggttccaatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 112 - DNA, 1776 bp, Synthetic construct
  source 1..1776: mol_type = other DNA
gcggccgcacgcgtttgaaggctgggatttgaacccaagtcttctgggaggtgagtcagacgccttgcgctcttcccccactgtgcctggagaacgttgctacctagggtccatgctgacaagtttatatagatgggaacataacagagagaaggcggaagttccaggtcacccagtcattagcccagctgggtcccccttctccttccacccagaaatagccctggcgcctgccccagaccagccctagcctctgggctccccgacgcttgtcaccagttacatctccagccacactggcccagacaacatctaagccctggttgtggtcgccgcaccccagaggaagacagggaacagctgaggcatgacccctcacctcactgggtgcacagcccccgcagaggcatgcatggattggagggcctttttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 113 - DNA, 1795 bp, Synthetic construct
  source 1..1795: mol_type = other DNA
gcggccgcacgcgtcttcctcaattaaagaccaaagacaatgataagacaactcaacttaaatcttgctaattgtgaaactaacgttcccttttctgggtagcaacatctcataatacagtcctcaacagcagactgatatatatgacattgccagatatccaaaaaaagtccacgggaacttacaaatgtaatccttcacggctttctcaaagaggtcatacacgtgctctctgtccaggccatcctgggccatgctgatctcgtcatgcagccactccagccagagctctacgagacaaaaatacttcccgcattaataccaaagggtaggctaagcgcagggactttaccaagagacatcagcatgcaaacgtttcatccatcttacacaacctggcgttgtttccagaaattcacttcttaaaacctgaactaagcacataggaggtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 114 - DNA, 1805 bp, Synthetic construct
  source 1..1805: mol_type = other DNA
gcggccgcacgcgttgctagtaagttggcccatccatgcggggctcagagacacagagcaggaccccacaccagcctgacacactacagtgaataaaaggagcccggcacacaaatggctgtacatatctgggtctagttctgctaggttctggtgccaagtccgctgagcccctacccaagctgctttcctggtcaccgtgcagtgactcacggtcaggccactggggctaaccacagtacaacagtcccagacaaaggcatcttgcggcatctgtcctctctgagggaagtggtgggcagtgggggaggcatgcagttcctctttggtttggaacatatttcattacttagaggccagcagtgaactgcctggggtggctagacctttctttctttctttctttttgttttgcattgtagcacaatgttaaatacaaaccagctttctacctcagtgtcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 115 - DNA, 1990 bp, Synthetic construct
  source 1..1990: mol_type = other DNA
gcggccgcacgcgtaaacaggcagtagctgagcattttcagctaaggatggagaagcccaagtctcctgggagacctcggcccagaacgtcagcaccactcacaagggcccaggcaccttgcaaaggcgggctccttgtcccttgggcccccagccactccccatggtttttcgaatcagacttaccgtgcccaacagagcatggggcagctgcctcagggcccagaagcaaacgactctgcctcagtgacagggccccagtccacactgccagacacttgtggtcccaatctgcccgccaacccccaacggcagggtgtggaagaaggaagcgagaatgaggttactcaggctggtcacgtgacgcgcccacacccccttgtttgtgacagctttggtgacaagtctgattaaagctgggtgggcagtgtgatggcggtttgttgggactgataacaatcttgaaagttcaaggaaggtatggggggtgcttactgtgaggggacctctacatgagacctcagagaagagtcacagccttgttctatgggcaactggagagaggcaaccagagagaacccctgccatggaggcaaaggccagacagaagggagggagggtctcaatggagatggagaatgggagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 116 - DNA, 1998 bp, Synthetic construct
  source 1..1998: mol_type = other DNA
gcggccgcacgcgtacacaagcctgtaatcccagctcagacaaggaagggacaggaggcttggggtgtgatggtctccctcagctacttagcaattgaaagaccaccccagactgcatgagaccctgtcttagaacagtgtggaccttcacggggcccagtcctggatgctcacaacgccagccagctctaacagagccacctgctccacaggatccaggcgttctggccgttgagctcctgggggccagggagacccacacctgcttgctctgggggaaaatggctcgaaggaagttggttaagaactagccttggagaaaggggaagtgagactgggcacatgctcctggtatttcagggctctgaactacggtcagacagtaggaagaccctttctctgaccttgaggcctcttgtctgagggagaccggctttccttaatgcataagcatgcctctgcctcttcatccatcagaggtctcagacccccaggcatcctcaggcagatgctgcccagcagcctaggcgatgcctgccttcagtgggcagtgagcattcttgttcctgcttacacagccaggaagcccgggttgtgtccccgactagagatcccttggtacacagtggcagccctgcccaggaattgtgttgagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 117 - DNA, 1846 bp, Synthetic construct
  source 1..1846: mol_type = other DNA
gcggccgcacgcgttcaaaaacaccgacatcgccaaggagctctgcctgccgcctgtgaaactgcactgctccagtgagtctgccccacctgccaggcagcttggatctgttcagagtctgctgttcccaccccacggtggccgggcagctgagacaggcagctcttcccccaccctgcgcttccccagtctgcctttgctgtctctgagtgtgtctgcagtgcctgaggccacagtgcactacctaccacactgccccacgagagccttcccacactgctaagcctcttccagaaagcagaagtaagacgtcaccctgacatgctcagctttctgcccccttccagctctcggcttctgcttcgcccatagcctcaccctcggtatcctctcagtactagccttccagtgtggggctgcagcaggaggcatgcagactgctgccaggtgacaagcactctctgcaataccctcactgttccagccagaaaggtgtgcaagtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 118 - DNA, 1984 bp, Synthetic construct
  source 1..1984: mol_type = other DNA
gcggccgcacgcgtaaatcagagatgcagctgaggcccagaaagggtaactgacttgatccagagaacccagccacacaaggggcagaggccagcccttcctcccagggttccctgtccatgaccagggcctacccaaggacccacctccatcatagatctctggaaaacctcagggtggagagctgggctccagagccctggactctcacccacatgctgcatctagggtccaatccaggcttcgtccctcactggccccctagagtaggccactcagcagtttccctaccataagaggaagatgggtacccgtgcaaggtgctaggctgccacacactctgccacatgggtgcacccctggaggttagaggcctgagggcaactgcaccatgtgagcagacagacaggaagagcctggtgagaagccagctccggtcagagcctttgaagacgctctggcaaaccacaagagacaaacagcgaagttaccacaggctgcccaggatcctgaggaagtctgagctcactggctttgggttccttccctcaaagcactcacccattcagccattcacccattcagccattcacccattcattcattcattcagccattcagccatctgtttattcattcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 119 - DNA, 1889 bp, Synthetic construct
  source 1..1889: mol_type = other DNA
gcggccgcacgcgttgccagttgaacaagatgctgtctcagagatgaacaagccatgatagaatatttcttctatcctcagagtttagttgagaaaattgaggcccagaaagggccagaacggtcctggcacacagtaggtgaccaacaagctggtcacttcctccctttctgctctccctccctccccttctgctctccctccctcccctcctgatcttgccagtcactagagagagaactctgtgtcacccatggatagaagttccacaaacaccgacaagggaaaactaaggggcaatttcattgaaacaacaagttattttgaaaactattggtccctgttacagttgattgtttggaaaccaagagatgtgtgtcccacacagaaaatttctgggaaatgttaccagtctttaaaaacatttggtaacagtaactgttttcaaaataattggctgtactacaggcagagggggaaaagaagtcaggagaagtgcactgtatatagaaaattggtaataaggggcatgaagtttcagagctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 120 - DNA, 1956 bp, Synthetic construct
  source 1..1956: mol_type = other DNA
gcggccgcacgcgtgtgggcagatcacgaggtcaggagttcgagaccagcctgcccaacatggtgaaaccctgttgctactacaaatacaaatacaaaaattagccgagtgtggtggtgtgcacctgtaatcccagctataaaggaggctgaggcagaagaattgcttgagcctgggaggctgggagccaggctcctgggagcctggaggttgcggtgagctgagatcaagtcactgcactccagcctgggcaaaagagagagactctgtctcaaaaaagaaaaaaaaaaggtgtccaagaaacctgggcaaagagcctgaactcctcagtggaggaggctcctagtgcaaggagtggaaaatgagggacagggaggggaagcattggtagaaggtgtacggaaccttcttggagttgagctttgatcttgaggaaaactggcagctgggttccaccaaacaacacactccagctattgtttccttgaactttggcctacattaatgaagaatctggggcaaatttttcacaggttcttccacactttatttttaaattttttgataactttattgagatataattcacataccgtacatacaattcacccagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 121 - DNA, 2139 bp, Synthetic construct
  source 1..2139: mol_type = other DNA
gcggccgcacgcgttcctgcgtccgcacgcgtagcctggtcctggaatagcacccagatatactggggaggttcatgaaagagaaaaggtctaagctgagagtggtcattagcctgagagatctgacctttcacagaaaagatttagtgtaattcggtaccgcgtgagtgtgcttataaaatgccaattcacctccactctcagaagacttttctaacatgcacctcatttcttactatgtatctggcctgaaataggagcattggctaaaggtgaggattcccaggggttgctactgtaattaaaacagaaagccagttcttggagacatggacactcagaacttgatggttaatgattatggttaatgccatctggttatgagcagtagaattctagggtgggggatactaagagtctgctgtaatatacatactactcaaatgtaagatgatatgcttatatttatgatcctttaacatcccaatctctggacctcagaagagaagactggctctgggagctaattaacatgcagtcaatatcccaagtctctctttgttcaagaaggcacaggctgaactcattaaactgccagcagaccagaaaaataagttatttgaaagccaacacaggaaaacttatttataaagctcagcagattttaaaagatcaaaacaaaacccattaataataggctgggcttgtagctaagtagagtgattgcgtgaagtcttgggttcaggttcgattcccagcgcagggtcaactttgtaaggcagtacacgcttgcaagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 122 - DNA, 2063 bp, Synthetic construct
  source 1..2063: mol_type = other DNA
gcggccgcacgcgttgcccagaagctatgtgaccacatgtaagtctcccattgcatggagatagaaaatttagagctagtagccctgcagaatctagttgcagaccccttcatgggccccgaatttcctgtcttgtcatcacagcaaagcgaggactgggaatggtctcagcatagtgctggctcagagccggcaaagttcctgctgttcttcaagggaaagttgtttgggttctttgtccagtaacttggtaacaagaaaactctcacgctggtcaaaaaaaaaaaaaaaaggaaaagcctcgggacccccatgccactcccagcttcctctgctccccccttgaaagcagagactagagcagtgggtggtattgggcctgttctgcccagaggctgaggagggtggcccgcccccaactgccagccagtacaagacaaaagacccttgacagttccctggggcggttcagctgtggcccggctggacaaagcaagggtacagggctcggtacagctctgcaaagtccttggggcttgtcttggcggagacttgggcttttgagaagctctgtccagggaagctctataaacccagacaacaaaatgacccctctcctcttacttgaagccagaagtcaaatggaagcaacaggccctggcctcaccctctttcaccccaaggccacatctctgaactagaggtggcagaagcaccttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 123 - DNA, 1939 bp, Synthetic construct
  source 1..1939: mol_type = other DNA
gcggccgcacgcgtcccctcacacacccacacatgtatatacactcacatcctgaagcacactcagacacatactaactctcagaaacacacttatccctcagagggggtaggaccagcctaggaaagagtccttctgggagaatggcatggctggtgactgccaaggggtaggcagaagcctaggactgggcagggagcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctctcttcttgcagggagggggtgggaaaccttccccataaccctggcatttcacccccagcccacacaggtggcttttggagaagctcaagtgccagaatttgacccgaaaactccaggttccacaacgccccctgcttccttcccaggccaggaagccttggaagtccagcccccaccaacctgtcccctagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 124 - DNA, 1661 bp, Synthetic construct
  source 1..1661: mol_type = other DNA
gcggccgcacgcgttgaggagaggaggccgagaaactgggctgagcaggacaaaacctcccctgtgaaccctgccaaggggaggggccccttattaaaacttactggggcatagccaggcccccctggagggagggggtctgaggggcagggaagggggctgggtggaggcaactaggactctggcctccagccagtcctcccctgggtggctgtttctgtggctgggctgtggttcacaccatctgtagcccttcagttgtccccttctccataccaaagtgctgtttcgcctagtcctgccctggtcccattcatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 125 - DNA, 1904 bp, Synthetic construct
  source 1..1904: mol_type = other DNA
gcggccgcacgcgtataaggagggtctttgtttctcttccctgggaaccgagcagcccctttctctgagggtagagttggggcatggctcaacagaacgacagtcatcctggctagtaagactcagaggctggccttcaaaggcttgagctcctgggtgactgagcagtggagaaacagaatcctgcccttgaattgcataaggagggtctttgtttctcttccctgggaaccgagcagcccctttctctgagggtagagttggggcatggctcaacagaacgacagtcatcctggctagtaagactcagaggctggccttcaaaggcttgagctcctgggtgactgagcagtggagaaacagaatcctgcccttgaattgcataaggagggtctttgtttctcttccctgggaaccgagcagcccctttctctgagggtagagttggggcatggctcaacagaacgacagtcatcctggctagtaagactcagaggctggccttcaaaggcttgagctcctgggtgactgagcagtggagaaacagaatcctgcccttgaattgcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 126 - DNA, 1964 bp, Synthetic construct
  source 1..1964: mol_type = other DNA
gcggccgcacgcgtactgagggataattctgatgtgtctattccagcagaacatttgaaagacgtgtccaattcaggccagagcgttgctctcagagcgaagggtatcaccatgctctagagttcagtctcctctgcacatacataaacacaagtgcatagcgccccaacgctgtttgtcccctcggcctcacagctctgccagttggcaaacccaagccaggaagttgtcaattccaattgcgtttaggccatgtagtcctattaaagaaggcctgctggttaaggggcgtcctctgtttccaaagcatgggaaaaaaaaaaaaaaggctatgcttagattggagctggcaactttcactgactagagttttcaaggagctatgaatgagtttaacaaaggaggtctgtggctagtttctggctgaagtccatgtcaacagtttttaaggtcacaggccacactaactaacatttattcccagcagaccctgttctaaacacaagctgtaagtagtacaaatttggaatttttctgtaatcccttgagatattcctgatccttccacatgtttctgctcaattaaaaacaattctcactttgcaccacaagagaggccagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 127 - DNA, 1719 bp, Synthetic construct
  source 1..1719: mol_type = other DNA
gcggccgcacgcgttgccaaagctggacaggctaagccagaggcaaggcaggggtgccagccctttgcccagggccagagggaggccaggaggaggtgcctgggctggttggcaccttctccctatccacgctctccctccagtcccaagtgacctttcccccagatcccagggtccaggcccgccctcggctggcaggtgtgggcacagaggcagctgggattggtcgcagctggcggaggcgcgtcccaggctccggcagaccgctggaacagctgagcagagcaggtggactgctgagatagaccagggacaccaggcagccacaggcctgtcagaccaggacccttaccctctagacatggcctcggtcccgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 128 - DNA, 1965 bp, Synthetic construct
  source 1..1965: mol_type = other DNA
gcggccgcacgcgttgtaggctgttcagtctgcagggttgggtggagtactccatctctttcctctctccccatcttcctagctcccagccctctcacattccaacctccattttcttgtcctctaccccattatttttctaactccttccccctccttttccttctctctagtttctccccctaccttttgctgacccattcccttcccccccattttctctgccaccttttacttaccccccccccccatcctcctccttttcttctgcttcgaactaattgcttaagaggtgaagaccaggtcggcttgtgaaaaccctgatccatcaccatggtgatgctaattcaagccgagttcaaaggaaatgactccagttaaatctaacaagctggagccaggcgcccagcaaggaccagtgccaggaggagggaggcccctcgcatgccagggcccagaaaaataggaaaaacaaccacggacgctcacacctctactctggtcctctcttggcatccttcagatctgcccaaacctaaagccgaccagtgagccccctgccttcagaccagactctacctaagtcatctcaaatagttggacatccagcaacatccccatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 129 - DNA, 1664 bp, Synthetic construct
  source 1..1664: mol_type = other DNA
gcggccgcacgcgtttgaaggctggaattagggggtgaggacccgcgtccttcgagccttgagggacatatgcaaagaattgctaattgcaactaaacgaggcctcggcgtgggggaaggggcagcccgggctgtcaggggctaccgcctctttgagtgcccgggtaaggccaggccagcccgctcttctcatgtaaagcagctttttgtctgggggagccagagctggggccaggggctggggaaggggcccaagcctttggcaagtttcttgttccccagaaagggagaagggtgccgaggcctcctcggagtgaggagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 130 - skipped

SEQ ID NO: 131 - DNA, 2093 bp, Synthetic construct
  source 1..2093: mol_type = other DNA
gcggccgcacgcgtctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagttctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagttctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 132 - skipped

SEQ ID NO: 133 - DNA, 1979 bp, Synthetic construct
  source 1..1979: mol_type = other DNA
gcggccgcacgcgtagcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctctagcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctctagcccaagagtagggggtggtgctgtgggccactgtgcagaggccaggactgtgaatgaagtgggcactgctcctgccccctcccaggcctgcctctggccaggccagcctccatcccggcattgtctcaggcatcttgggcagggttcccagagctggctgaaccaggggcctggccaaacagccctcccctgccctcccattcctctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 134 - DNA, 1712 bp, Synthetic construct
  source 1..1712: mol_type = other DNA
gcggccgcacgcgtaagcaaagcccaggcatcctatgggaggtctggctaaaagccagggtcctacttgcccccacgctccacccaactcaatccctgccccttagacctcctcccaccagggggctggctacaaagcaaagcccaggcatcctatgggaggtctggctaaaagccagggtcctacttgcccccacgctccacccaactcaatccctgccccttagacctcctcccaccagggggctggctacaaagcaaagcccaggcatcctatgggaggtctggctaaaagccagggtcctacttgcccccacgctccacccaactcaatccctgccccttagacctcctcccaccagggggctggctacagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 135 - DNA, 1851 bp, Synthetic construct
  source 1..1851: mol_type = other DNA
gcggccgcacgcgtatgtgcatacctgcctctgagaaggaccaaagtcagatgcactctcaatgtaagggcttgggggtggaacacctcaagttccacacacaccccaatctcctacaggagtttcctttagaggacctgtctcctccaaggggctctgttagaccaagtgggaaagtctctaggaactgacttcagaggtttggggctgtgccagtcccgcggcgcactctgccacggaggagacctggagctcctgggttggccaccttgctccctgcctcctcccacctcctccccagcaggttatcagcatgaaaacaggaaggctggcagtgctattaataaaacatgaagcagagatctgccccatcaggcagcggaggcaggcggcagctcggcagccccgagggagggagagaacccccgcatctgggcctgggcaggggtattctcttctcacagatgccacaccccaggagccacctccattttgcgggactgctgtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 136 - protein, 6 aa, Synthetic construct
HHHHHH

SEQ ID NO: 137 - protein, 8 aa, Synthetic construct
DYKDDDDK

SEQ ID NO: 138 - protein, 8 aa, Synthetic construct
DLYDDDDK

SEQ ID NO: 139 - protein, 15 aa, Synthetic construct
GLNDIFEAQKIEWHE

SEQ ID NO: 140 - protein, 26 aa, Synthetic construct
KRRWKKNFIAVSAANRFKKISSSGAL

SEQ ID NO: 141 - protein, 9 aa, Synthetic construct
YPYDVPDYA

SEQ ID NO: 142 - protein, 10 aa, Synthetic construct
EQKLISEEDL

SEQ ID NO: 143 - protein, 8 aa, Synthetic construct
WRHPQFGG

SEQ ID NO: 144 - protein, 8 aa, Synthetic construct
WSHPQFEK

SEQ ID NO: 145 - protein, 13 aa, Synthetic construct
SLAELLNAGLGGS

SEQ ID NO: 146 - protein, 8 aa, Synthetic construct
TQDPSRVG

SEQ ID NO: 147 - protein, 14 aa, Synthetic construct
GKPIPNPLLGLDST

SEQ ID NO: 148 - protein, 4 aa, Synthetic construct
SAQA

SEQ ID NO: 149 - protein, 11 aa, Synthetic construct
SDGTLAVPFKA

SEQ ID NO: 150 - skipped

SEQ ID NO: 151 - protein, 7 aa, Synthetic construct
NRGTEWD

SEQ ID NO: 152 - protein, 7 aa, Synthetic construct
QAVRTSL

SEQ ID NO: 153 - protein, 7 aa, Synthetic construct
TLAVPFK

SEQ ID NO: 154 - protein, 7 aa, Synthetic construct
DSPAHPS

SEQ ID NO: 155 - DNA, 490 bp, Homo sapiens
  source 1..490: mol_type = other DNA
aggagcgaacatggtatgcataattgaaacactcctccttgtttgaaatgttgtctcctcctctgcagtcctgcggcaaagacaggcacacaggctttctctgtggattaattggtcaggaaagacactttttatattgtcaagtggcacttaagccattaattcttgactgtgaaactgcttttcctgggcaatgctctttaaggaaaaacttattcagtccagtgactggtgactaagggaacgatggagcacagggaattgggcgggatctagactcctaataatgcctccttagccaatgaaaagcatttcctattgagacccccaagagttcccctggccgtcggctccagctcggacttcaggcctttttgtgtcctgtttgctaaaggcatgcgggctacagcattcaagagagggagtcgttaacaaagggaaagagataaatgtaaataagctcacatttacagaatgagcggtttgcagt

SEQ ID NO: 156 - DNA, 563 bp, Homo sapiens
  source 1..563: mol_type = other DNA
ccattcttttgaagttggcactttgatttctagatggttccccaacacaggttcttctcctcctcatcattatagctgccttgaaatttgagctggaagggaacattctgagacccagattgttaaatgtcttttccaaagtcatgcaataaattaaatggcaaagccagggcagtttcttgactcagtacagggtattttctttcattctttactcttgagactttagaactgttggtactgctttaaaattcatggcaagaactggtcacttttgtaattaacacctccttataatacatttgttttgtttgcttagccagctagaaactacatggagtctgtgctttaaaaagcctgccgaagtccttattctctgttttggtattatgtgcatgaaccaccaattggttccttctcaccttacacttgatgaagatgtctttctttcaacatctttctctattgctccccatcttctcttgctctatttatgatcagctgtctgtttctaaatagactttgtggtcacccatttctttttgtgccagctcctatccact

SEQ ID NO: 157 - DNA, 711 bp, Homo sapiens
  source 1..711: mol_type = other DNA
gattaatgacactctaatggtacagaacagaaaattctctgtctctaagatcatctaaaccaaagacatgtacacaactacctataatgtaggccagaccacagaaatgctaagtgatgctttaaagtcttctgaaaaataatgccttaactaaacaaagtccccctacatccagagtatgaatgatgctgctattacttatttctactgtcttccatctattttatagtgttctttgatccattctttgcattcttcttgtttgaaagtaatattcttgatagtttaataatgagacccctcctttgttcctagatgccttggcggctaatcaacaaaagccattgaaagacttaatcctctaagcttggcaccacatcagtctggaagtagtttacatgcagagtcctttggagtaaatactcattagaggttctgttggctcatttggtcacccacattttgcagtgggtggcctagttggcatcctcattattttctttttccactcctgaaaataatccagatgtagcaacctgacactactttctttggtactccaaatccattgttatgaagggtccattaatttgtgatgctagctaatatgtccaaataaataaaggcctcaggccagtaatgctgtaataaatcttttactaatacacttttttcagtgcaacctattgaatgcaattcaatggcttcaag

SEQ ID NO: 158 - DNA, 743 bp, Homo sapiens
  source 1..743: mol_type = other DNA
gtgtcccggtgacaacctctttccaccactctgcaaggcttccaagcaactccagtagtctctgtggatgaatcagagcacccaggctgacagcactgtgcacctctgcttctgggatgacggtggaacacagggacgtctgggcctcttaggtcagggtcctcttgctcaagggacctaaccactgctcttcacgctagcaccagaccaacacgtgctgcaaggacactggatggtcgctgcactgaatgcagctgcccagagccttatctcatcagtgggcactgaggactgatgtcagcagccctaagacaaagcttcctttgggaggacagtagaagagggggtgcagcaacaagtgggccattgcatgtgttgtttatcaacatttggatgacggagattttggccaatttcagaaagacactcatattttgtagaaacacagtacagtgctctcatggagagaaaaacatcgcacaaattagggtggagaagaatctgatgcagtgcccttcagagacacagctgagtgggatgatgcaaccctgagcttggagccacctgatctaaattcgagcccacacttagccacttaggagctgtgtagtcttggataagtcactcaaaccctctggtctccattttctcacaacaaatctattctccaaggctcagttttcccattctcaaaatgggatactattaacttgccttgcccacttcacaagggaataccgtaa

SEQ ID NO: 159 - DNA, 123 bp, Mus musculus
  source 1..123: mol_type = other DNA
gaaatcttaacacaccatgaagattgtccaagcgccaagccttccgttctggactaaattactttgaagtggctcaggacgagcagtggtcaattttaactctatagactggacagaagaggc

SEQ ID NO: 160 - DNA, 369 bp, Synthetic construct
  source 1..369: mol_type = other DNA
gaaatcttaacacaccatgaagattgtccaagcgccaagccttccgttctggactaaattactttgaagtggctcaggacgagcagtggtcaattttaactctatagactggacagaagaggcgaaatcttaacacaccatgaagattgtccaagcgccaagccttccgttctggactaaattactttgaagtggctcaggacgagcagtggtcaattttaactctatagactggacagaagaggcgaaatcttaacacaccatgaagattgtccaagcgccaagccttccgttctggactaaattactttgaagtggctcaggacgagcagtggtcaattttaactctatagactggacagaagaggc

SEQ ID NO: 161 - DNA, 1848 bp, Synthetic construct
  source 1..1848: mol_type = other DNA
gcggccgcacgcgtaggagcgaacatggtatgcataattgaaacactcctccttgtttgaaatgttgtctcctcctctgcagtcctgcggcaaagacaggcacacaggctttctctgtggattaattggtcaggaaagacactttttatattgtcaagtggcacttaagccattaattcttgactgtgaaactgcttttcctgggcaatgctctttaaggaaaaacttattcagtccagtgactggtgactaagggaacgatggagcacagggaattgggcgggatctagactcctaataatgcctccttagccaatgaaaagcatttcctattgagacccccaagagttcccctggccgtcggctccagctcggacttcaggcctttttgtgtcctgtttgctaaaggcatgcgggctacagcattcaagagagggagtcgttaacaaagggaaagagataaatgtaaataagctcacatttacagaatgagcggtttgcagtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 162 - DNA, 1921 bp, Synthetic construct
  source 1..1921: mol_type = other DNA
gcggccgcacgcgtccattcttttgaagttggcactttgatttctagatggttccccaacacaggttcttctcctcctcatcattatagctgccttgaaatttgagctggaagggaacattctgagacccagattgttaaatgtcttttccaaagtcatgcaataaattaaatggcaaagccagggcagtttcttgactcagtacagggtattttctttcattctttactcttgagactttagaactgttggtactgctttaaaattcatggcaagaactggtcacttttgtaattaacacctccttataatacatttgttttgtttgcttagccagctagaaactacatggagtctgtgctttaaaaagcctgccgaagtccttattctctgttttggtattatgtgcatgaaccaccaattggttccttctcaccttacacttgatgaagatgtctttctttcaacatctttctctattgctccccatcttctcttgctctatttatgatcagctgtctgtttctaaatagactttgtggtcacccatttctttttgtgccagctcctatccactgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 163 - DNA, 2615 bp, Synthetic construct
  source 1..2615: mol_type = other DNA
gcggccgcacgcgtgattaatgacactctaatggtacagaacagaaaattctctgtctctaagatcatctaaaccaaagacatgtacacaactacctataatgtaggccagaccacagaaatgctaagtgatgctttaaagtcttctgaaaaataatgccttaactaaacaaagtccccctacatccagagtatgaatgatgctgctattacttatttctactgtcttccatctattttatagtgttctttgatccattctttgcattcttcttgtttgaaagtaatattcttgatagtttaataatgagacccctcctttgttcctagatgccttggcggctaatcaacaaaagccattgaaagacttaatcctctaagcttggcaccacatcagtctggaagtagtttacatgcagagtcctttggagtaaatactcattagaggttctgttggctcatttggtcacccacattttgcagtgggtggcctagttggcatcctcattattttctttttccactcctgaaaataatccagatgtagcaacctgacactactttctttggtactccaaatccattgttatgaagggtccattaatttgtgatgctagctaatatgtccaaataaataaaggcctcaggccagtaatgctgtaataaatcttttactaatacacttttttcagtgcaacctattgaatgcaattcaatggcttcaaggagctcgggctgggcataaaagtcagggcagagccatctattgcttacatttgcttctgggatccagatctttcgaagctagcgctaccggtcgccaccatggtctccaagggcgaggagctgttcaccggggtggtgcccatcctggtcgagctggacggcgatgtcaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggctctggtgctaccaacttctcactgttgaaacaggcaggggatgtagaggagaatccagggcctggtgctagtggagactacaaagaccatgacggagattataaagatcatgacatcgattacaaggatgacgatgacaagtccggactcagatctggaggctccggaggcccagagccagcgaagtctgctcccgccccgaaaaagggctccaagaaggcggtgactaaggcgcagaagaaaggcggcaagaagcgcaagcgcagccgcaaggagagctattccatctatgtgtataaggttctgaagcaggtccaccctgacaccggcatttcgtccaaggccatgggcatcatgaactcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaagtaatgaggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 164 - DNA, 2647 bp, Synthetic construct
  source 1..2647: mol_type = other DNA
gcggccgcacgcgtgtgtcccggtgacaacctctttccaccactctgcaaggcttccaagcaactccagtagtctctgtggatgaatcagagcacccaggctgacagcactgtgcacctctgcttctgggatgacggtggaacacagggacgtctgggcctcttaggtcagggtcctcttgctcaagggacctaaccactgctcttcacgctagcaccagaccaacacgtgctgcaaggacactggatggtcgctgcactgaatgcagctgcccagagccttatctcatcagtgggcactgaggactgatgtcagcagccctaagacaaagcttcctttgggaggacagtagaagagggggtgcagcaacaagtgggccattgcatgtgttgtttatcaacatttggatgacggagattttggccaatttcagaaagacactcatattttgtagaaacacagtacagtgctctcatggagagaaaaacatcgcacaaattagggtggagaagaatctgatgcagtgcccttcagagacacagctgagtgggatgatgcaaccctgagcttggagccacctgatctaaattcgagcccacacttagccacttaggagctgtgtagtcttggataagtcactcaaaccctctggtctccattttctcacaacaaatctattctccaaggctcagttttcccattctcaaaatgggatactattaacttgccttgcccacttcacaagggaataccgtaagagctcgggctgggcataaaagtcagggcagagccatctattgcttacatttgcttctgggatccagatctttcgaagctagcgctaccggtcgccaccatggtctccaagggcgaggagctgttcaccggggtggtgcccatcctggtcgagctggacggcgatgtcaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagggctctggtgctaccaacttctcactgttgaaacaggcaggggatgtagaggagaatccagggcctggtgctagtggagactacaaagaccatgacggagattataaagatcatgacatcgattacaaggatgacgatgacaagtccggactcagatctggaggctccggaggcccagagccagcgaagtctgctcccgccccgaaaaagggctccaagaaggcggtgactaaggcgcagaagaaaggcggcaagaagcgcaagcgcagccgcaaggagagctattccatctatgtgtataaggttctgaagcaggtccaccctgacaccggcatttcgtccaaggccatgggcatcatgaactcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaagtaatgaggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 165 - DNA, 1721 bp, Synthetic construct
  source 1..1721: mol_type = other DNA
gcggccgcacgcgtgaaatcttaacacaccatgaagattgtccaagcgccaagccttccgttctggactaaattactttgaagtggctcaggacgagcagtggtcaattttaactctatagactggacagaagaggcgaaatcttaacacaccatgaagattgtccaagcgccaagccttccgttctggactaaattactttgaagtggctcaggacgagcagtggtcaattttaactctatagactggacagaagaggcgaaatcttaacacaccatgaagattgtccaagcgccaagccttccgttctggactaaattactttgaagtggctcaggacgagcagtggtcaattttaactctatagactggacagaagaggcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 166 - DNA, 623 bp, Mus musculus
  source 1..623: mol_type = other DNA
aagcatgaatggcctctcaggatggaaggggggcggggagagaagaaagccggtcagaggcagcagttgcttttaagaatatgtaagatttctagtttctgaggtgctgtgttatagcagctttaagcaagacctaacaatttgttaaatacttcatctccaaacagtggtgggtactacagtttcatgagcagcagataatagattctgtttattcgccaacaataccgacactgtttcgccccggactgcaagtggctgagccatctggctcctatctccacagtaaaacattgtccgtgacacaaaaaatgactacatccccaggacaaactagtgtgacctctaaataaagccttcctctgaagctatgatatcacactgcacaggggacaccagcacaattgaatctggtcaccaaaagcccaagtgagataggactatgtaaatgggattttataggagacgttttgtcctctgtgcaaaccatcgagatccgtccacagcactgaaacatctttttcattcaccctgaggacaacagtgtgcttctttctggggggttggcagtcctctttctccggccccaggccgaggaagcacacatccgtgtcccagaggtg

SEQ ID NO: 167 - DNA, 418 bp, Mus musculus
  source 1..418: mol_type = other DNA
tggtgcgacagaaatatggctaaagctagttttgttttaaatagaacatggatttctatgaaagaattatattgataaattattagtagcaagttaagataaattgttaacatttgtctactttatgcactaggcacaaatacttattaatgaaacccctcatgtgtgaagattatttatttatgtttctcaaacaaaaatggtattgtatccgattcactgggtgtgctttgtccagtctgattttctcattagcaattctctgcatgattaaatgctaaatacacagctgtgtaaagcctcaactggtagtaaccagtagtgcctaatactgctgtcttaaaggaataacaatatagtcaaatactggatgtacaagctttaaggccccattcagctgaaagacctggggttatca

SEQ ID NO: 168 - DNA, 492 bp, Mus musculus
  source 1..492: mol_type = other DNA
tggctctggcaaaggatggagattttttttttaacctattcttttgagaataaggagggtctttgtttctcttccctgggaaccgagcagcccctttctctgagggtagagttggggcatggctcagcagaacgacagtcatcctggctagtaagactcagaggctggccttcaaaggcttgagctcctgggtgactgagcagtggagaaacagaatcctgcccttgaattgctccccaggcgggctttatgcagtctggggaagcaagggatgccctgtgattcttaaagaaactggtataattttgcactgcatagcagactcccaagacacacagccttttccaggaggagttccttagaggggaaggggataggttgcaatggtctgcttagcctcccgaaggctgcagaagctgctaagacaggggtaactgaaagatagactctgtgaggttgaacagcaatctttatgacctgaccccctaggca

SEQ ID NO: 169 - DNA, 691 bp, Homo sapiens
  source 1..691: mol_type = other DNA
ccccaagcaaggtcaacagcagcttcaattacaagacaaacttaacaagagttgctataaaccaagtactctgtattgacttaagaacaggctccactccacatattgccactagcaggattgtacaggaatgcatattgtaaaataaaggaaggggtgagcttttttctttgccagaatttgcgagtgcacagcgactcctcattcacctctctccaaccagctagccgctcagctcaattcaccccacacaaaggctggagcctagacctcaatggaccgagtgaaacatgttcaaaactaggctctctattgtgactgaatttcttaacatcttttcaaaaagcggagaatgccttgaggctaaaggaagaaacaggctaatggtgaattgggaattctgagcaaatttcagagccctttcctcctagcttttgaggttgaaagcaagctctttcctttcaagtttcaaagtcctttttcctcccgcagtgtcacagaaggatttgaaaagaaggtaattgtgctcgcagtctccctgatcagagcttacgtcctatttctggtatttcggaatacttcttgcaataatagtgcatatagctcaatcccttaaccggcctgcactctgcaattgctcattaaatgaacaattgcgggtataaaatgccttttatgttcaaggtctgga

SEQ ID NO: 170 - DNA, 292 bp, Mus musculus
  source 1..292: mol_type = other DNA
gatgggtgggaccaggaagggactgggcaggagctcattttgggataggggaggggacagctgaggggttacagatggagtaaagcaaagcccaggcatcctatgggaggtctggctaaaagccagggtcctacttgcccccacgctccacccaactcaatccctgccccttagacctcctcccaccagggggctggctacaccccagtaagttttaacaaggcacccctccccttggcaggactcacagggaaggttttgtcttgctcagcccagtttctccagcgcctct

SEQ ID NO: 171 - DNA, 322 bp, Mus musculus
  source 1..322: mol_type = other DNA
agatatctgacaatgagcagaagcttgtatagagtgtataaaacaagtttccccttcttattttctgctttaaaacgcacaatctcccactcccagcctcttctgtccagtctatagagttaaaattgaccactgctcgtcctgagccacttcaaagtaatttagtccagaacggaaggcttggcgcttggacaatcttcatggtgtgttaagatttctatggcaattggcaagcaagacgttcccatctgaatatgtctcaaagaaagccacttattgacactaagttggctatcatcaaagacttcatcttccctctgca

(19) United States
(12) Patent Application Publication
(10) Pub. No.: US 2026/0250707 A1
(43) Pub. Date: Aug. 27, 2026

(54) ARTIFICIAL EXPRESSION CONSTRUCTS FOR MODULATING GENE EXPRESSION IN NON-NEURONAL CENTRAL NERVOUS SYSTEM CELLS

(71) Applicant: Allen Institute, Seattle, WA (US)

(72) Inventors: Edward Sebastian Lein, Mercer Island, WA (US); Boaz P. Levi, Seattle, WA (US); Refugio Martinez, Seattle, WA (US); John K. Mich, Seattle, WA (US); Ximena Opitz-Araya, Seattle, WA (US); Jonathan Ting, Lake Forest Park, WA (US)

(73) Assignee: Allen Institute, Seattle, WA (US)

(21) Appl. No.: 18/874,771

(22) PCT Filed: Jun. 13, 2023
(86) PCT No.: PCT/US23/68369

§ 371 Date: Dec. 13, 2024

Related U.S. Application Data
(60) Provisional application No. 63/351,631, filed on Jun. 13, 2022.

(51) Int. Cl. C12N 15/85; C12N 15/86

(52) U.S. Cl. CPC C12N 15/8509; C12N 15/86; C12N 2750/14143; C12N 2830/15; C12N 2830/48

(57) ABSTRACT

To fully understand the biology of the brain, different cell types need to be distinguished and defined and, to further study them, artificial expression constructs that can label and perturb them need to be identified, in mouse, recombinase driver lines have been used to great effect to label cell populations that share marker gene expression. However, the creation, maintenance, and use of such lines that label cell types with high specificity can be costly, frequently requiring triple transgenic crosses, which yield a low frequency of experimental animals. Artificial expression constructs for modulating gene expression in targeted central nervous system cell types are described. The artificial expression constructs can be used to express synthetic genes or modify gene expression in non-neuronal cells including oligodendrocytes, microglia, astrocytes, or endothelial cells.

### CROSS-REFERENCE TO RELATED APPLICATIONS

This application is a U.S. National Phase Patent Application based on International Patent Application No. PCT/US2023/068369, filed Jun. 13, 2023, which claims priority to U.S. Provisional Patent Application No. 63/351,631 filed Jun. 13, 2022, each of which is incorporated herein by reference in its entirety as if fully set forth herein.

### STATEMENT REGARDING FEDERALLY SPONSORED RESEARCH OR DEVELOPMENT

This invention was made with government support under MH114126 and MH120095 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3CJ3599.XML. The file is 278,528 bytes, was created on Dec. 10, 2024, and is being submitted electronically via Patent Center.

### FIELD OF THE DISCLOSURE

The current disclosure provides artificial expression constructs for modulating gene expression in targeted central nervous system cell types. The artificial expression constructs can be used to express synthetic genes or modify gene expression in non-neuronal central nervous system cells including oligodendrocytes, microglia, astrocytes, and endothelial cells.

### BACKGROUND OF THE DISCLOSURE

To fully understand the biology of the brain, different cell types need to be distinguished and defined and, to further study them, artificial expression constructs that can label and perturb them need to be identified. In mouse, recombinase driver lines have been used to great effect to label cell populations that share marker gene expression. However, the creation, maintenance, and use of such lines that label cell types with high specificity can be costly, frequently requiring triple transgenic crosses, which yield a low frequency of experimental animals. Furthermore, those tools require germline transgenic animals and thus are not applicable to humans.

### SUMMARY OF THE DISCLOSURE

The current disclosure provides artificial expression constructs that drive gene expression in targeted central nervous system cell populations. Targeted central nervous system cell populations include: non-neuronal brain cells including glial cells which reside in the parenchyma and can be subdivided into oligodendrocytes, microglia, astrocytes, and oligodendrocyte progenitors; and pericytes, endothelial, and ependymal cells, which reside in other cellular compartments.

Particular embodiments of the artificial expression constructs utilize the following enhancers to drive gene expression within targeted central nervous system cell populations as follows (targeted cell population/enhancer(s)):
oligodendrocytes/eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, and eHGT_363h;
microglia/eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, and eHGT_734m;
astrocytes/eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, and eHGT_358h;
L1 interlaminar astrocytes/eHGT_312h, eHGT_313h, eHGT_315h, and eHGT_316h; and
endothelial cells/eHGT_603m.

In particular embodiments, the artificial enhancer elements include a concatenated core of an enhancer. Examples include a core or concatenated core of eHGT_374m, eHGT_375m, eHGT_380h, eHGT_387m, eHGT_390h, eHGT_400m, eHGT_401h, eHGT_409h, eHGT_410h, and/or eHGT_641m. These artificial enhancer elements can provide higher levels and more rapid onset of transgene expression compared to a single full length original (native) enhancer.

Particular embodiments of the enhancer cores include Core_eHGT_641m, core2_eHGT_400m, core2_eHGT_401h, core2_eHGT_409h, core2_eHGT_410h, core1_eHGT_387m, core2_eHGT_390h, core2_eHGT_380h, core2_eHGT_374m, and core2_eHGT_375m. In particular embodiments, these cores are concatenated and have 2, 3, 4, 5, 6, 7, 8, 9, or 10 copies of the core sequence. In particular embodiments, a three-copy concatemer of the selected enhancer cores include 3xCore_eHGT_641m, 3xcore2_eHGT_400m, 3xcore2_eHGT_401h, 3xcore2_eHGT_409h, 3xcore2_eHGT_410h, 3xcore1_eHGT_387m, 3xcore2_eHGT_390h, 3xcore2_eHGT_380h, 3xcore2_eHGT_374m, and 3xcore2_eHGT_375m.

Particular embodiments provide artificial expression constructs including the features of vectors described herein including vectors: CN3062, CN3063, CN2979, CN2498, CN2677, CN2505, CN2499, CN4004, CN3575, CN4254, CN4255, CN4256, CN2806, CN2807, CN2808, CN2809, CN2812, CN2813, CN2678, CN2679, CN2680, CN2681, CN2833, CN2834, CN2835, CN2836, CN2627, CN2675, CN2344, CN3060, CN3061, CN3098, CN2493, CN3568, CN2495, CN2496, CN2827, CN2846, CN3572, CN4253, CN4257, CN2410, CN2345, CN2346, CN4268, CN4522, and CN4523.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIGS. 1A-1D. Fluorescent images of sagittal sections of mouse brain transduced by CN3060 virus after intravenous delivery. Virus is packaged with PHP.eB capsid. Black is SYFP expression. Images are montages. (1A) Whole brain, (1B) visual cortex (VISp), (1C) deep cerebellar nucleus (DCN), and (1D) cerebellar cortex (Bergmann glia).

FIGS. 2A-2D. (2A) Fluorescent image of mouse brain in sagittal section transduced by CN2499 virus after intravenous delivery. SYFP2 transgene is driven by enhancer eHGT_403h. Virus is packaged with PHP.eB capsid. Images are montages. The brain is co-stained with propidium iodide (PI) and 4′,6-diamidino-2-phenylindole (DAPI). Note abundant reporter labeling oligodendrocytes enriched in mid and hindbrain structures and in other white matter tracts. (2B-2D) Fluorescent images of mouse non-brain transduced by CN2499 virus after intravenous delivery. SYFP transgene (black) is driven by enhancer eHGT_403h. Virus is packaged with PHP.eB capsid. Images are montages. (2B) Lumbar spinal cord, (2C) liver, and (2D) heart are shown. Strong oligodendrocyte expression is seen in spinal cord, but little expression is observed in heart and liver.

FIGS. 3A, 3B. Fluorescent image of mouse brain in sagittal section transduced by CN3061 virus after intravenous delivery. SYFP2 transgene (black) is driven by enhancer eHGT_389m. Virus is packaged with PHP.eB capsid. Images are montages. (3A) Whole brain, (3B) visual cortex (VISp-broken rectangle in FIG. 4A) are shown. Note reporter expression in sparse cells with astrocyte morphology.

FIGS. 4A, 4B. Fluorescent image of mouse brain in sagittal section transduced by CN3098 virus after intravenous delivery. SYFP2 transgene (black) is driven by enhancer eHGT_380h. Virus is packaged with PHP.eB capsid. Images are montages. (4A) Whole brain, (5B) visual cortex (VISp-broken rectangle in FIG. 4A) are shown. Note reporter expression in sparse cells with astrocyte morphology.

FIGS. 5A, 5B. Fluorescent image of mouse brain in sagittal section transduced by CN2498 virus after intravenous delivery. SYFP2 transgene (black) is driven by enhancer eHGT_395m. Virus is packaged with PHP.eB capsid. Images are montages. (5A) Whole brain, (5B) visual cortex (VISp-broken rectangle in FIG. 5A) are shown. Note reporter expression in cells with oligodendrocyte morphology and enrichment in white matter tracts.

FIGS. 6A, 6B. Fluorescent image of mouse brain in sagittal section transduced by CN2677 virus after intravenous delivery. SYFP2 transgene (black) is driven by enhancer eHGT_401h. Virus is packaged with PHP.eB capsid. Images are montages. (6A) Whole brain, (6B) visual cortex (VISp-broken rectangle in FIG. 6A) are shown. Note reporter expression in cells with oligodendrocyte morphology and enrichment in white matter tracts.

FIGS. 7A, 7B. Widespread astrocyte-specific labeling in the mouse brain with CN3572. (7A) Epifluorescence image of native SYFP2 expression in mouse brain sagittal section at 24 days post retroorbital injection of 2.5E+11 viral genomes of AAV vector CN3572 serotype PHP.eB (rAAV-3xcore2_eHGT_390 h-minBglobin-SYFP2-WPRE3-BGHpA). Abbreviations: CTX, cortex; STR, striatum; HPF, hippocampal formation; TH, thalamus; CB, cerebellum; BS, brainstem. Scale bar=1 mm. (7B) Expanded view of the boxed region in (7A) showing finer details of cellular labeling of astrocytes in CTX region. Scale bar=200 microns.

FIGS. 8A, 8B. Widespread oligodendrocyte-specific labeling in the mouse brain with CN3575. (8A) Epifluorescence image of native SYFP2 expression in mouse brain sagittal section at 27 days post retroorbital injection of 5.0E+11 viral genomes of AAV vector CN3575 serotype PHP.eB (rAAV-3xcore2_eHGT_400m-minBglobin-SYFP2-WPRE3-BGHpA). Scale bar=1 mm. (8B) Expanded view of the boxed region in (8A) showing finer details of cellular labeling of astrocytes in CTX region. Scale bar=250 microns.

FIG. 9. Sequences supporting the disclosure including eHGT_409h (SEQ ID NO: 1); eHGT_410h (SEQ ID NO: 2); eHGT_361h (SEQ ID NO: 3); eHGT_395m (SEQ ID NO: 4); eHGT_401h (SEQ ID NO: 5); eHGT_391h (SEQ ID NO: 6); eHGT_403h (SEQ ID NO: 7); eHGT_641m (SEQ ID NO: 166); Core_eHGT_641m (SEQ ID NO: 8); 3xCore_eHGT_641m (SEQ ID NO: 9); eHGT_400m (SEQ ID NO: 167); core2_eHGT_400m (SEQ ID NO: 10); 3xcore2_eHGT_400m (SEQ ID NO: 11); core2_eHGT_401h (SEQ ID NO: 12); 3xcore2_eHGT_401h (SEQ ID NO: 13); core2_eHGT_409h (SEQ ID NO: 14); 3xcore2_eHGT_409h (SEQ ID NO: 15); core2_eHGT_410h (SEQ ID NO: 16); 3xcore2_eHGT_410h (SEQ ID NO: 17); eHGT_331h (SEQ ID NO: 18); eHGT_332h (SEQ ID NO: 19); eHGT_333h (SEQ ID NO: 20); eHGT_335h (SEQ ID NO: 21); eHGT_334h (SEQ ID NO: 22); eHGT_360h (SEQ ID NO: 23); eHGT_562h (SEQ ID NO: 24); eHGT_563h (SEQ ID NO: 25); eHGT_564h (SEQ ID NO: 26); eHGT_565h (SEQ ID NO: 27); eHGT_566m (SEQ ID NO: 28); eHGT_567m (SEQ ID NO: 29); eHGT_568m (SEQ ID NO: 30); eHGT_569m (SEQ ID NO: 31); eHGT_734m (SEQ ID NO: 32); eHGT_312h (SEQ ID NO: 33); eHGT_313h (SEQ ID NO: 34); eHGT_377m (SEQ ID NO: 35); eHGT_389m (SEQ ID NO: 36); eHGT_380h (SEQ ID NO: 37); eHGT_374h (SEQ ID NO: 38); eHGT_387m (SEQ ID NO: 168); core1_eHGT_387m (SEQ ID NO: 39); 3xcore1_eHGT_387m (SEQ ID NO: 40); eHGT_385h (SEQ ID NO: 41); eHGT_386h (SEQ ID NO: 42); eHGT_378m (SEQ ID NO: 43); eHGT_642m (SEQ ID NO: 44); eHGT_390h (SEQ ID NO: 169); core2_eHGT_390h (SEQ ID NO: 47); 3xcore2_eHGT_390h (SEQ ID NO: 48); core2_eHGT_380h (SEQ ID NO: 51); 3xcore2_eHGT_380h (SEQ ID NO: 52); eHGT_374m (SEQ ID NO: 170); core2_eHGT_374m (SEQ ID NO: 53); 3xcore2_eHGT_374m (SEQ ID NO: 54); eHGT_603m (SEQ ID NO: 55); eHGT_315h (SEQ ID NO: 155); eHGT_316h (SEQ ID NO: 156); eHGT_358h (SEQ ID NO: 157); eHGT_363h (SEQ ID NO: 158); eHGT_375m (SEQ ID NO: 171); core2_eHGT_375m (SEQ ID NO: 159); 3xcore2_eHGT_375m (SEQ ID NO: 160); Beta-Globin Minimal Promoter (pBGmin/minBGlobin/minBGprom) (SEQ ID NO: 56); minCMV Promoter (SEQ ID NO: 57); Mutated minCMV Promoter (Sacl RE site removed) (SEQ ID NO: 58); minRho Promoter (SEQ ID NO: 59); minRho* Promoter (SEQ ID NO: 60); Hsp68 minimal Promoter (proHsp68) (SEQ ID NO: 61); SYFP2 (SEQ ID NO: 62); EGFP (SEQ ID NO: 63); Optimized Flp recombinase (FIpO) (SEQ ID NO: 64); Improved Cre recombinase (iCre) (SEQ ID NO: 65); SP10 insulator (SP10ins) (SEQ ID NO: 66); 3xSP10ins (SEQ ID NO: 67); 3XFLAG (SEQ ID NO: 68); 10aa (SEQ ID NO: 69); H2B (SEQ ID NO: 70); WPRE3 (SEQ ID NO: 71); WPRE (SEQ ID NO: 72); BGHpA (SEQ ID NO: 73); hGHpA (SEQ ID NO: 74); P2A (SEQ ID NO: 75); T2A (SEQ ID NO: 76); E2A (SEQ ID NO: 77); F2A (SEQ ID NO: 78); Exemplary Plasmid Backbone 1-Left ITR (SEQ ID NO: 79); Exemplary Plasmid Backbone 1-Right ITR (SEQ ID NO: 80); Exemplary Plasmid Backbone 2-Left ITR (SEQ ID NO: 81); Exemplary Plasmid Backbone 2-Right ITR (SEQ ID NO: 82); PHP.eB capsid (SEQ ID NO: 83); AAV9 VP1 capsid protein (SEQ ID NO: 84); tet-Transactivator version 2 (tTA2) (SEQ ID NO: 85); GTPase HRas [Homo sapiens] (SEQ ID NO: 86); Substance P is position 58-68 of Protachykinin-1 [Homo sapiens] (SEQ ID NO: 87); Oxytocin is position 20-28 of Oxytocin-neurophysin 1 [Homo sapiens] (SEQ ID NO: 88); GCaMP6m (SEQ ID NO: 89); GCaMP6s (SEQ ID NO: 90); GCaMP6f (SEQ ID NO: 91); CN3062 (SEQ ID NO: 92); CN3063 (SEQ ID NO: 93); CN2979 (SEQ ID NO: 94); CN2498 (SEQ ID NO: 95); CN2677 (SEQ ID NO: 96); CN2505 (SEQ ID NO: 97); CN2499 (SEQ ID NO: 98); CN4004 (SEQ ID NO: 99); CN3575 (SEQ ID NO: 100); CN4254 (SEQ ID NO: 101); CN4255 (SEQ ID NO: 102); CN4256 (SEQ ID NO: 103); CN2806 (SEQ ID NO: 104); CN2807 (SEQ ID NO: 105); CN2808 (SEQ ID NO: 106); CN2809 (SEQ ID NO: 107); CN2812 (SEQ ID NO: 108); CN2813 (SEQ ID NO: 109); CN2678 (SEQ ID NO: 110); CN2679 (SEQ ID NO: 111); CN2680 (SEQ ID NO: 112); CN2681 (SEQ ID NO: 113); CN2833 (SEQ ID NO: 114); CN2834 (SEQ ID NO: 115); CN2835 (SEQ ID NO: 116); CN2836 (SEQ ID NO: 117); CN2627 (SEQ ID NO: 118); CN2675 (SEQ ID NO: 119); CN2344 (SEQ ID NO: 120); CN3060 (SEQ ID NO: 121); CN3061 (SEQ ID NO: 122); CN3098 (SEQ ID NO: 123); CN2493 (SEQ ID NO: 124); CN3568 (SEQ ID NO: 125); CN2495 (SEQ ID NO: 126); CN2496 (SEQ ID NO: 127); CN2827 (SEQ ID NO: 128); CN2846 (SEQ ID NO: 129); CN3572 (SEQ ID NO: 131); CN4253 (SEQ ID NO: 133); CN4257 (SEQ ID NO: 134); CN2410 (SEQ ID NO: 135); CN2345 (SEQ ID NO: 161); CN2346 (SEQ ID NO: 162); CN4522 (SEQ ID NO: 163); CN4523 (SEQ ID NO: 164); and CN4268 (SEQ ID NO: 165).

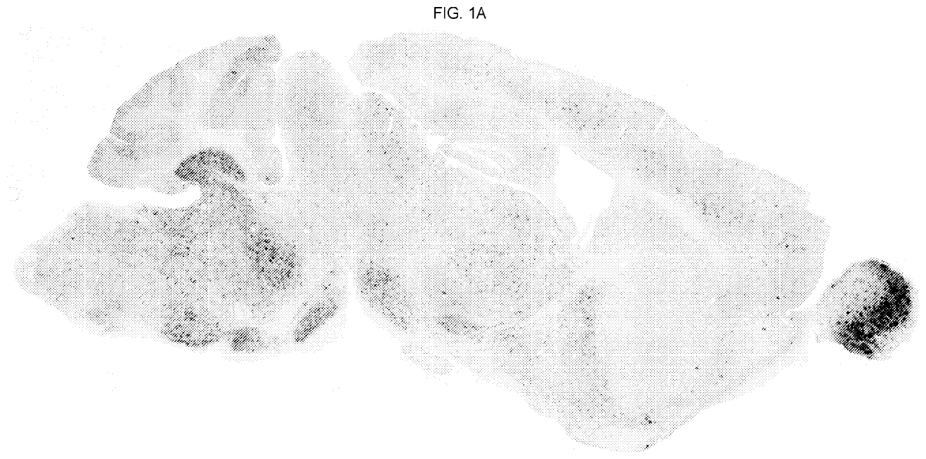

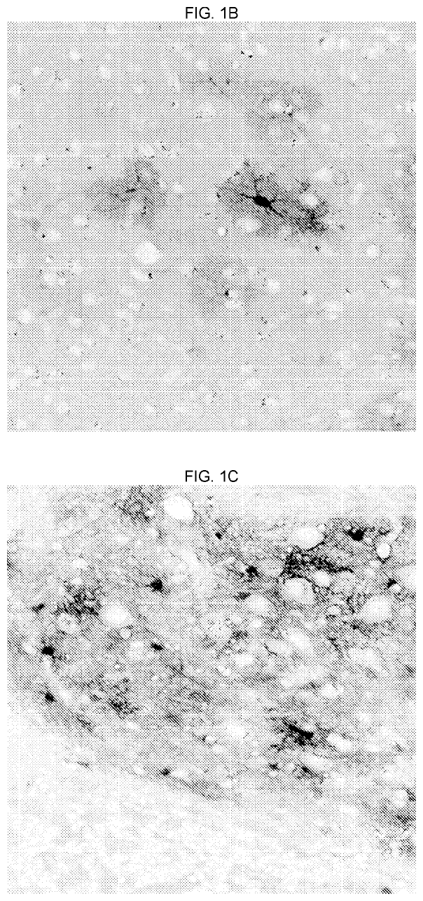

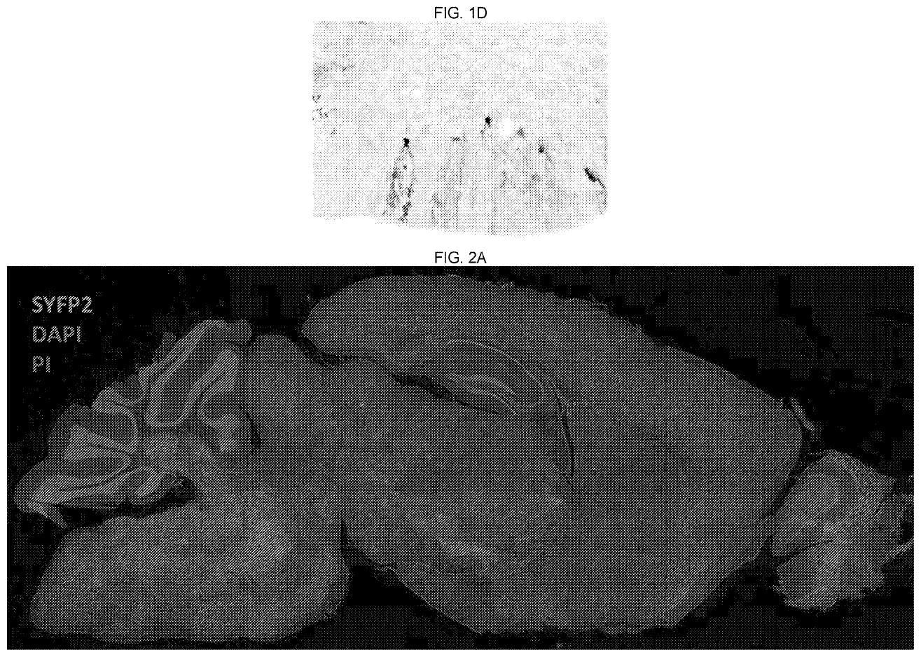

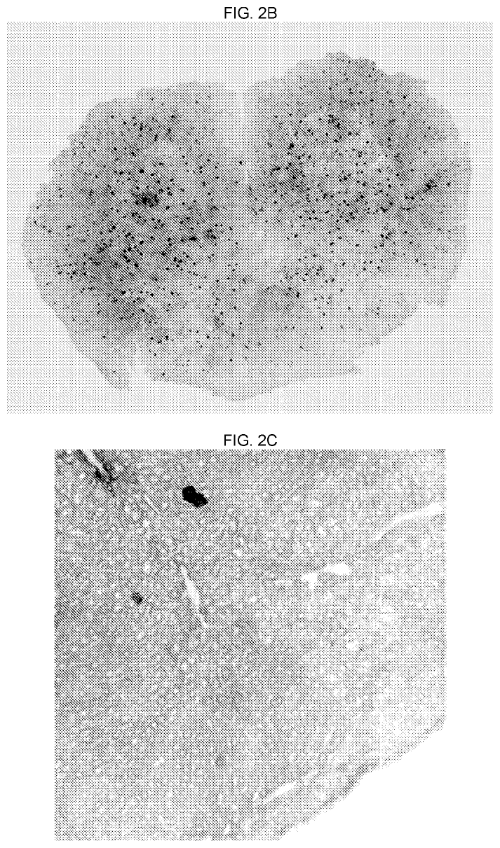

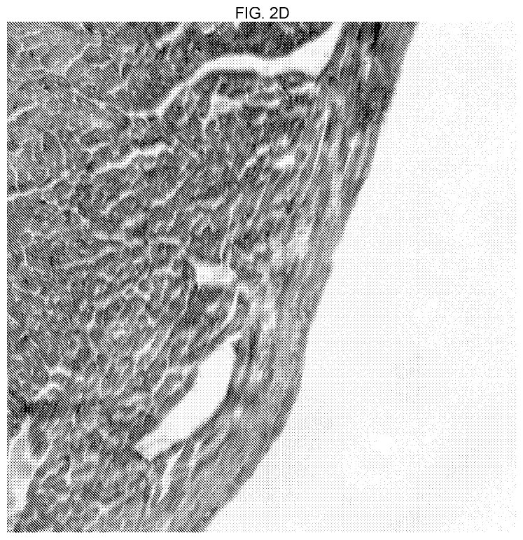

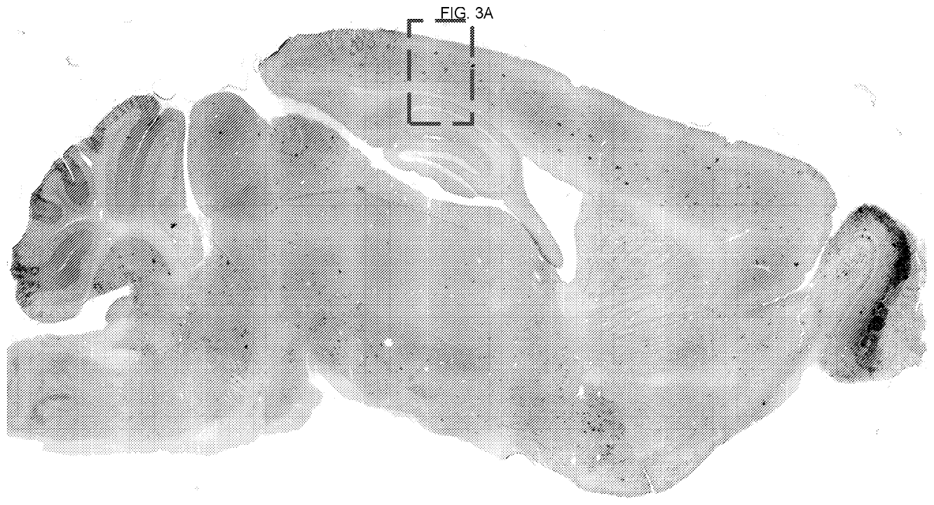

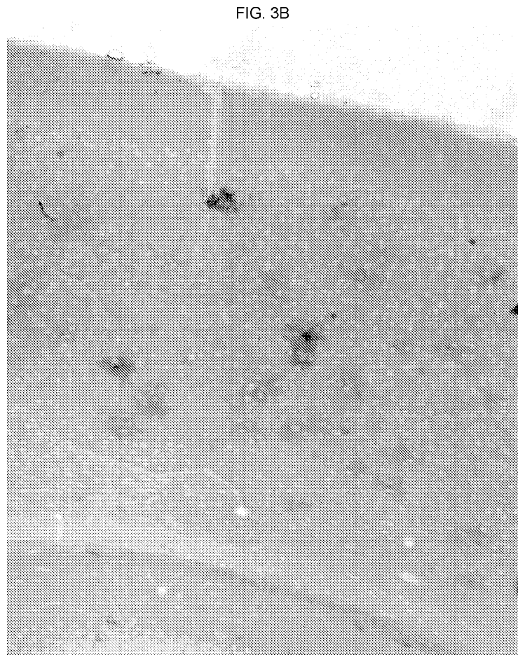

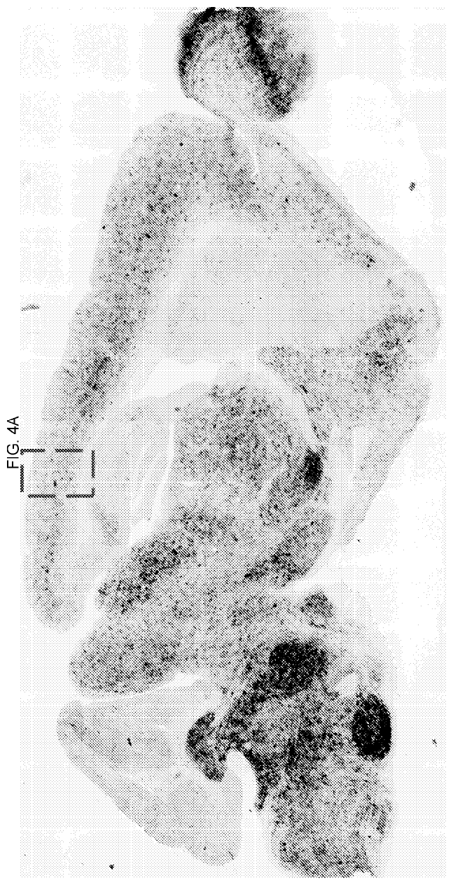

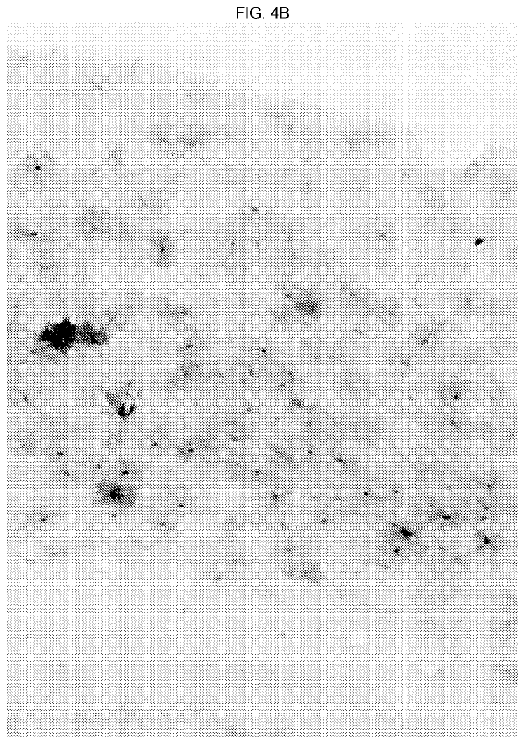

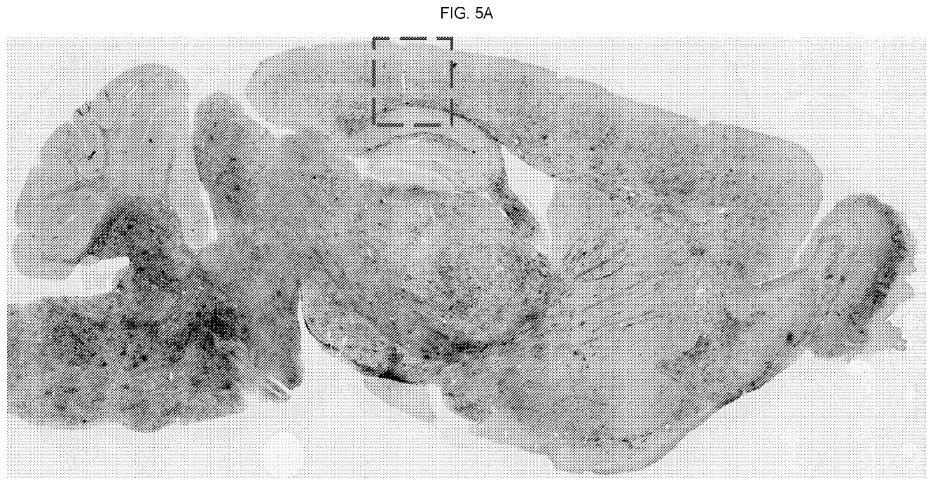

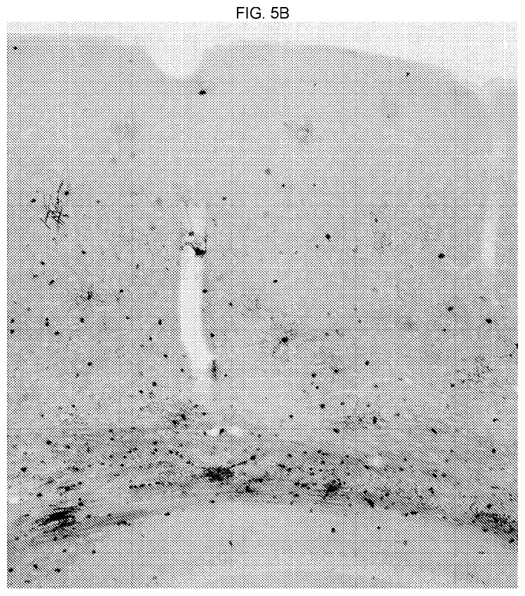

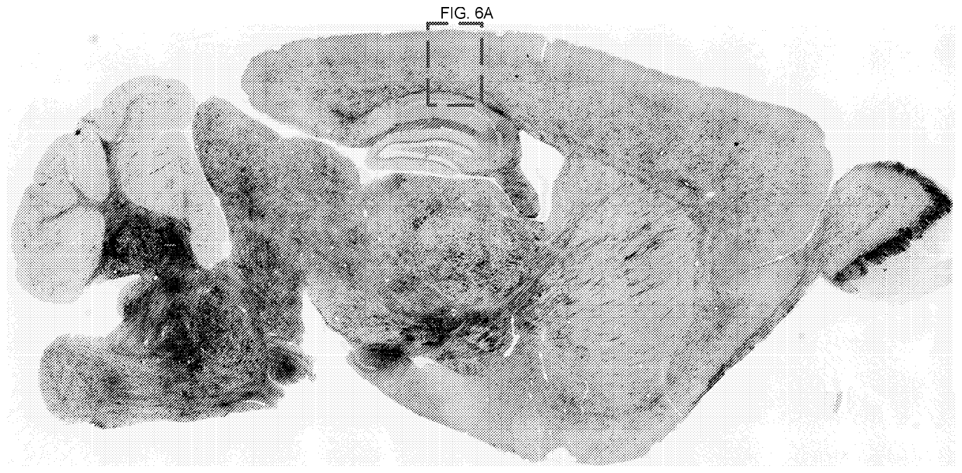

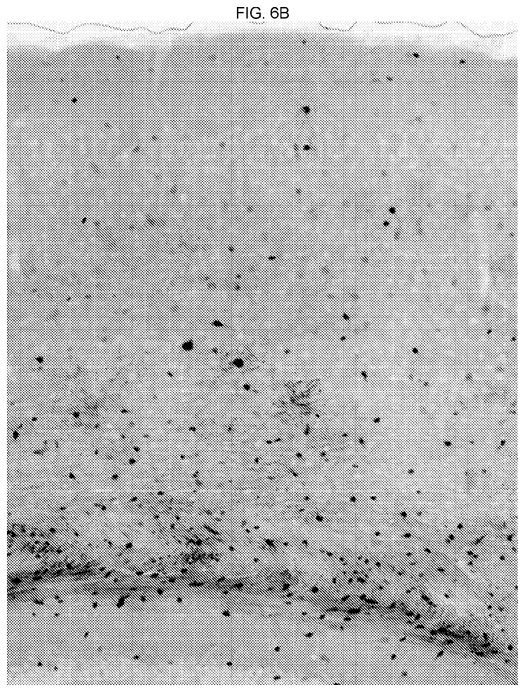

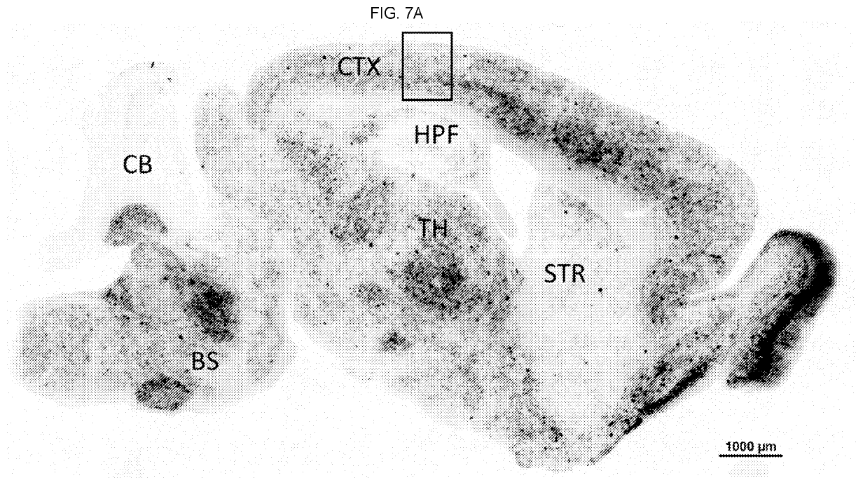

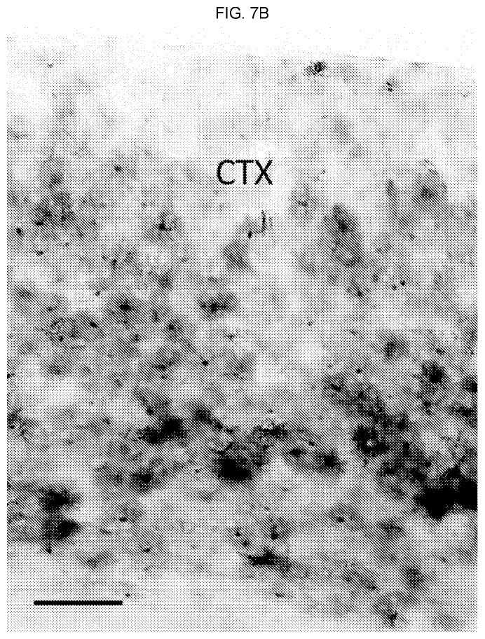

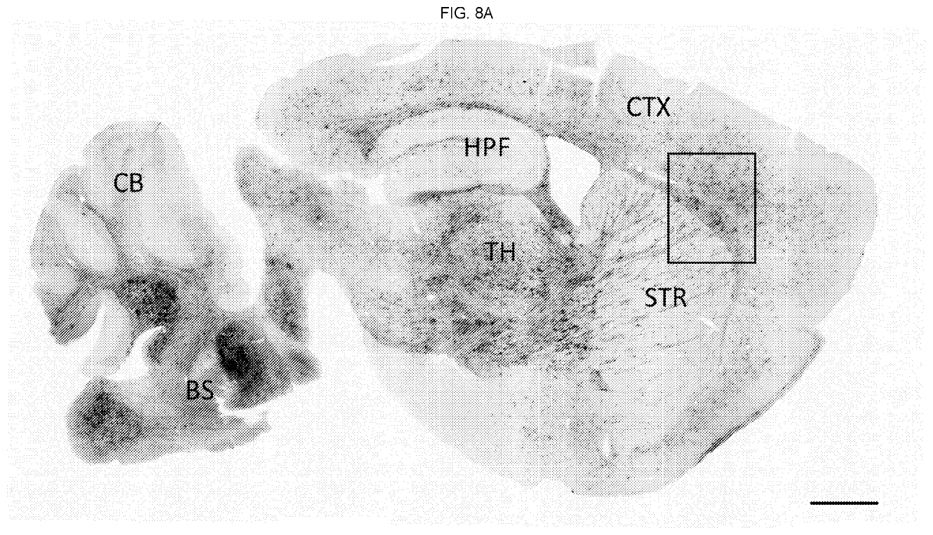

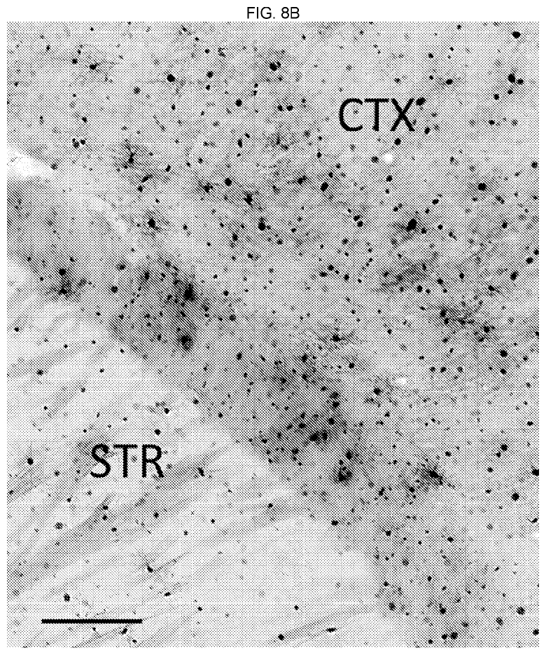

### DETAILED DESCRIPTION

To fully understand the biology of the brain, different cell types need to be distinguished and defined and, to further study them, artificial expression constructs that can label and perturb them need to be identified (Tasic, Curr. Opin. Neurobiol. 50, 242-249 (2018); Zeng & Sanes, Nat. Rev. Neurosci. 18, 530-546 (2017)). In mouse, recombinase driver lines have been used to great effect to label cell populations that share marker gene expression (Daigle et al., Cell 174, 465-480.e22 (2018); Taniguchi, et al., Neuron 71, 995-1013 (2011); Gong et al., J. Neurosci. 27, 9817-9823 (2007)). However, the creation, maintenance, and use of such lines that label cell types with high specificity can be costly, frequently requiring triple transgenic crosses, which yield a low frequency of experimental animals. Furthermore, those tools require germline transgenic animals and thus are not applicable to humans.

The current disclosure provides artificial expression constructs that drive gene expression in targeted central nervous system cell populations. Targeted central nervous system cell populations include: non-neuronal brain cells including glial cells which reside in the parenchyma and can be subdivided into oligodendrocytes, microglia, astrocytes, and oligodendrocyte progenitors; and pericytes, endothelial, and ependymal cells, which reside in other cellular compartments.

Particular embodiments of the artificial expression constructs utilize the following enhancers to drive gene expression within targeted central nervous system cell populations as follows (targeted cell population/enhancer(s)):
oligodendrocytes/eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, and eHGT_363h;
microglia/eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, and eHGT_734m;
astrocytes/eHGT_377m, eHGT_389m, eHGT_380h, eHGT_374h, eHGT_386h, 3xcore1_eHGT_387m, eHGT_385h, eHGT_378m, eHGT_642m, 3xcore2_eHGT_390h, 3xcore2_eHGT_380h, 3xcore2_eHGT_374m, 3xcore2_eHGT_375m, and eHGT_358h;
L1 interlaminar astrocytes/eHGT_312h, eHGT_313h, eHGT_315h, and eHGT_316h; and
endothelial cells/eHGT_603m.

In particular embodiments, the artificial enhancer elements include a concatenated core of an enhancer. Examples include a core or concatenated core of eHGT_374m, eHGT_375m, eHGT_380h, eHGT_387m, eHGT_390h, eHGT_400m, eHGT_401h, eHGT_409h, eHGT_410h, and/or eHGT_641m. These artificial enhancer elements can provide higher levels and more rapid onset of transgene expression compared to a single full length original (native) enhancer.

Particular embodiments of the enhancer cores include Core_eHGT_641m, core2_eHGT_400m, core2_eHGT_401h, core2_eHGT_409h, core2_eHGT_410h, core1_eHGT_387m, core2_eHGT_390h, core2_eHGT_380h, core2_eHGT_374m, and core2_eHGT_375m. In particular embodiments, these cores are concatenated and have 2, 3, 4, 5, 6, 7, 8, 9, or 10 copies of the core sequence. In particular embodiments, a three-copy concatemer of the selected enhancer cores include 3xCore_eHGT_641m, 3xcore2_eHGT_400m, 3xcore2_eHGT_401h, 3xcore2_eHGT_409h, 3xcore2_eHGT_410h, 3xcore1_eHGT_387m, 3xcore2_eHGT_390h, 3xcore2_eHGT_380h, 3xcore2_eHGT_374m, and 3xcore2_eHGT_375m.

Particular embodiments provide artificial expression constructs including the features of vectors described herein including vectors: CN3062, CN3063, CN2979, CN2498, CN2677, CN2505, CN2499, CN4004, CN3575, CN4254, CN4255, CN4256, CN2806, CN2807, CN2808, CN2809, CN2812, CN2813, CN2678, CN2679, CN2680, CN2681, CN2833, CN2834, CN2835, CN2836, CN2627, CN2675, CN2344, CN3060, CN3061, CN3098, CN2493, CN3568, CN2495, CN2496, CN2827, CN2846, CN3572, CN4253, CN4257, CN2410, CN2345, CN2346, CN4268, CN4522, and CN4523.

Aspects of the disclosure are now described with the following additional options and detail: (i) Artificial Expression Constructs & Vectors for Targeted Expression of Genes in Targeted Cell Types; (ii) Compositions for Administration (iii) Cell Lines Including Artificial Expression Constructs; (iv) Transgenic Animals; (v) Methods of Use; (vi) Kits and Commercial Packages; (vii) Exemplary Embodiments; and (viii) Closing Paragraphs. These headings are provided for organization purposes only and do not limit the scope or interpretation of the disclosure.

(i) Artificial Expression Constructs & Vectors for Targeted Expression of Genes in Targeted Cell Types. Artificial expression constructs disclosed herein include (i) an enhancer sequence that leads to targeted expression of a coding sequence within a targeted central nervous system cell type, (ii) a coding sequence that is expressed, and (iii) a promoter. The artificial expression construct can also include other regulatory elements if necessary or beneficial.

In particular embodiments, an “enhancer” or an “enhancer element” is a cis-acting sequence that increases the level of transcription associated with a promoter and can function in either orientation relative to the promoter and the coding sequence that is to be transcribed and can be located upstream or downstream relative to the promoter or the coding sequence to be transcribed. There are art-recognized methods and techniques for measuring function(s) of enhancer element sequences. Particular examples of enhancer sequences utilized within artificial expression constructs disclosed herein include eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, and eHGT_603m.

In particular embodiments, a targeted central nervous system cell type enhancer is an enhancer that is uniquely or predominantly utilized by the targeted central nervous system cell type. A targeted central nervous system cell type enhancer enhances expression of a gene in the targeted central nervous system. In certain embodiments, a targeted central nervous system cell type enhancer is also a targeted central nervous system type enhancer that enhances expression of a gene in the targeted central nervous system and does not substantially direct expression of genes in other non-targeted cell types, thus having cell type specific transcriptional activity.

When a heterologous coding sequence operatively linked to an enhancer disclosed herein leads to expression in a targeted cell type, it leads to expression of the administered heterologous coding sequence in the intended cell type.

When a heterologous coding sequence is selectively expressed in selected cells, it leads to expression of the administered heterologous coding sequence in the intended cell type and is not substantially expressed in other cell types, as explained in additional detail below. In particular embodiments, not substantially expressed in other cell types is less than 50% expression in a reference cell type as compared to a targeted cell type; less than 40% expression in a reference cell type as compared to a targeted cell type; less than 30% expression in a reference cell type as compared to a targeted cell type; less than 20% expression in a reference cell type as compared to a targeted cell type; or less than 10% expression in a reference cell type as compared to a targeted cell type. In particular embodiments, a reference cell type refers to non-targeted cells. The non-targeted cells can be within the same anatomical structure as the targeted cells and/or can project to a common anatomical area. In particular embodiments, a reference cell type is within an anatomical structure that is adjacent to an anatomical structure that includes the targeted cell type. In particular embodiments, a reference cell type is a non-targeted cell with a different gene expression profile than the targeted cells.

In particular embodiments, the product of the coding sequence may be expressed at low levels in non-selected cell types, for example at less than 1% or 1%, 2%, 3%, 5%, 10%, 15% or 20% of the levels at which the product is expressed in selected cells. In particular embodiments, the targeted central nervous system cell type is the only cell type that expresses the right combination of transcription factors that bind an enhancer disclosed herein to drive gene expression. Thus, in particular embodiments, expression occurs exclusively within the targeted cell type.

In particular embodiments, targeted cell types (e.g., non-neuronal) can be identified based on transcriptional profiles, such as those described in Tasic et al., Nature 563, 72-78 (2018) and Hodge et al., Nature 573, 61-68 (2019). For reference, the following description of cell types and distinguishing features is also provided:

Non-neuronal Subclasses:
Astrocytes: Neuroectoderm-derived glial cells which express the marker Aqp4 and often GFAP, but do not express neuronal marker SNAP25. They can have a distinct star-shaped morphology and are involved in metabolic support of other cells in the brain. Multiple astrocyte morphologies are observed in mouse and human
Oligodendrocytes: Neuroectoderm-derived glial cells, which express the marker Sox10. This category includes oligodendrocyte precursor cells (OPCs). Oligodendrocytes are the subclass that is primarily responsible for myelination of neurons.
VLMCs: Vascular leptomeningeal cells (VLMCs) are part of the meninges that surround the outer layer of the cortex and express the marker genes Lum and Col1a1.
Pericytes: Blood vessel-associated cells that express the marker genes Konj8 and Abcc9. Pericytes wrap around endothelial cells and are important for regulation of capillary blood flow and are involved in blood-brain barrier permeability.
SMCs: Specialized smooth-muscle cells which are blood vessel-associated cells that express the marker gene Acta2. SMCs cover arterioles in the brain and are involved in blood-brain barrier permeability.
Endothelial cells: Cells that line blood vessels of the brain. Endothelial cells express the markers Tek and PDGF-B.
Microglia: hematopoietic-derived immune cells, which are brain-resident macrophages, and perivascular macrophages (PVMs) that may be transitionally associated with brain tissue or included as a biproduct of brain dissection methods. Microglia are known to express Cx3cr1, Tmem119, and PTPRC (CD45).

In particular embodiments, a coding sequence is a heterologous coding sequence that encodes an effector element. An effector element is a sequence that is expressed to achieve, and that in fact achieves, an intended effect. Examples of effector elements include reporter genes/proteins and functional genes/proteins.

Exemplary reporter genes/proteins include those expressed by Addgene ID #s 83894 (pAAV-hDIx-Flex-dTomato-Fishell_7), 83895 (pAAV-hDIx-Flex-GFP-Fishell_6), 83896 (pAAV-hDIx-GiDREADD-dTomato-Fishell-5), 83898 (pAAV-mDIx-ChR2-mCherry-Fishell-3), 83899 (pAAV-mDIx-GCaMP6f-Fishell-2), 83900 (pAAV-mDIx-GFP-Fishell-1), and 89897 (pcDNA3-FLAG-mTET2 (N500)). Exemplary reporter genes particularly can include those which encode an expressible fluorescent protein, or expressible biotin; blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen), CopGFP, AceGFP, avGFP, ZsGreenl, Oregon Green™ (Thermo Fisher Scientific)); Luciferase; orange fluorescent proteins (mOrange, mKO, Kusabira-Orange, Monomeric Kusabira-Orange, mTangerine, tdTomato, dTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

GFP is composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostability and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ¿), also known as its optical cross section of 9.13×10−21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm.

Exemplary functional molecules include functioning ion transporters, cellular trafficking proteins, enzymes, transcription factors, neurotransmitters, calcium reporters, channelrhodopsins, guide RNA, nucleases, microRNA, or designer receptors exclusively activated by designer drugs (DREADDs).

Ion transporters are transmembrane proteins that mediate transport of ions across cell membranes. These transporters are pervasive throughout most cell types and important for regulating cellular excitability and homeostasis. Ion transporters participate in numerous cellular processes such as action potentials, synaptic transmission, hormone secretion, and muscle contraction. Many important biological processes in living cells involve the translocation of cations, such as calcium (Ca2+), potassium (K+), and sodium (Na+) ions, through such ion channels. In particular embodiments, ion transporters include voltage gated sodium channels (e.g., SCN1A), potassium channels (e.g., KCNQ2), and calcium channels (e.g., CACNA1C)).

Exemplary enzymes, transcription factors, receptors, membrane proteins, cellular trafficking proteins, signaling molecules, and neurotransmitters include enzymes such as lactase, lipase, helicase, alpha-glucosidase, and aromatic I-amino acid decarboxylase (AADC), amylase; transcription factors such as SP1, AP-1, Heat shock factor protein 1, C/EBP (CCAA-T/enhancer binding protein), and Oct-1; receptors such as transforming growth factor receptor beta 1, platelet-derived growth factor receptor, epidermal growth factor receptor, vascular endothelial growth factor receptor, and interleukin 8 receptor alpha; membrane proteins, cellular trafficking proteins such as clathrin, dynamin, caveolin, Rab-4A, and Rab-11A; signaling molecules such as nerve growth factor (NGF), glial cell line-derived neurotrophic factor (GDNF), platelet-derived growth factor (PDGF), transforming growth factor β (TGFβ), epidermal growth factor (EGF), GTPase and HRas; and neurotransmitters such as cocaine and amphetamine regulated transcript, substance P, oxytocin, and somatostatin.

In particular embodiments, functional molecules include reporters of cell function and states such as calcium reporters. Intracellular calcium concentration is an important predictor of numerous cellular activities, which include neuronal activation, muscle cell contraction and second messenger signaling. A sensitive and convenient technique to monitor the intracellular calcium levels is through the genetically encoded calcium indicator (GECI). Among the GECIs, green fluorescent protein (GFP) based calcium sensors named GCaMPs are efficient and widely used tools. The GCaMPs are formed by fusion of M13 and calmodulin protein to N- and C-termini of circularly permutated GFP. Some GCaMPs yield distinct fluorescence emission spectra (Zhao et al., Science, 2011, 333 (6051): 1888-1891). Exemplary GECIs with green fluorescence include GCaMP3, GCaMP5G, GCaMP6s, GCaMP6m, GCaMP6f, jGCaMP7s, jGCaMP7c, jGCaMP7b, jGCaMP7f, jGCaMP8s, jGCaMP8m, and jGCaMP8f. Furthermore, GECIs with red fluorescence include jRGECO1a and jRGECO1b. AAV products containing GECIs are commercially available. For example, Vigene Biosciences provides AAV products including AAV8-CAG-GCaMP3 (Cat. No: BS4-CX3AAV8), AAV8-Syn-FLEX-GCaMP6s-WPRE (Cat. No: BS1-NXSAAV8), AAV8-Syn-FLEX-GCaMP6s-WPRE (Cat. No: BS1-NXSAAV8), AAV9-CAG-FLEX-GCaMP6m-WPRE (Cat. No: BS2-CXMAAV9), AAV9-Syn-FLEX-jGCaMP7s-WPRE (Cat. No: BS12-NXSAAV9), AAV9-CAG-FLEX-jGCaMP7f-WPRE (Cat. No: BS12-CXFAAV9), AAV9-Syn-FLEX-jGCaMP7b-WPRE (Cat. No: BS12-NXBAAV9), AAV9-Syn-FLEX-jGCaMP7c-WPRE (Cat. No: BS12-NXCAAV9), AAV9-Syn-FLEX-NES-jRGECO1a-WPRE (Cat. No: BS8-NXAAAV9), and AAV8-Syn-FLEX-NES-jRCaMP1b-WPRE (Cat. No: BS7-NXBAAV8).

In particular embodiments calcium reporters include the genetically encoded calcium indicators GECI, NTnC; Myosin light chain kinase, GFP, Calmodulin chimera; Calcium indicator TN-XXL; BRET-based auto-luminescent calcium indicator; and/or Calcium indicator protein OeNL (Ca2+)-18u).

In particular embodiments, functional molecules include modulators of neuronal activity like channelrhodopsins (e.g., channelrhodopsin-1, channelrhodopsin-2, and variants thereof). Channelrhodopsins are a subfamily of retinylidene proteins (rhodopsins) that function as light-gated ion channels. In addition to channelrhodopsin 1 (ChR1) and channelrhodopsin 2 (ChR2), several variants of channelrhodopsins have been developed. For example, Lin et al. (Biophys J, 2009, 96 (5): 1803-14) describe making chimeras of the transmembrane domains of ChR1 and ChR2, combined with site-directed mutagenesis. Zhang et al. (Nat Neurosci, 2008, 11 (6): 631-3) describe VChR1, which is a red-shifted channelrhodopsin variant. VChR1 has lower light sensitivity and poor membrane trafficking and expression. Other known channelrhodopsin variants include the ChR2 variant described in Nagel, et al., Proc Natl Acad Sci USA, 2003, 100 (24): 13940-5), ChR2/H134R (Nagel, G., et al., Curr Biol, 2005, 15 (24): 2279-84), and ChD/ChEF/ChIEF (Lin, J. Y., et al., Biophys J, 2009, 96 (5): 1803-14), which are activated by blue light (470 nm) but show no sensitivity to orange/red light. Additional variants are described in Lin, Experimental Physiology, 2010, 96.1:19-25; Knopfel et al., The Journal of Neuroscience, 2010, 30 (45): 14998-15004; and Mardinly et al., Nat Neurosci. 2018, 21 (6): 881-893).

In particular embodiments, functional molecules include DNA and RNA editing tools such CRISPR/Cas (e.g., guide RNA and a nuclease, such as Cas, Cas9 or cpf1). Functional molecules can also include engineered Cpf1s such as those described in US 2018/0030425, US 2016/0208243, WO/2017/184768 and Zetsche et al. (2015) Cell 163:759-771; single gRNA (see e.g., Jinek et al. (2012) Science 337:816-821; Jinek et al. (2013) eLife 2: e00471; Segal (2013) eLife 2: e00563) or editase, guide RNA molecules, microRNA, or homologous recombination donor cassettes.

In particular embodiments, functional molecules include a localizing cassette. In particular embodiments, localizing cassettes are used to localize a molecule (e.g., a vector, a protein, a sensor) to a specific subcellular compartment such as the soma, axon, or dendrite(s) of a neuron. In particular embodiments, localizing cassettes include a soma tag (e.g., soma (EE-RR)) to localize at the soma; an axon tag (e.g., derived from GAP43) or synaptophysin (sy) to localize at the axon; hydrophobic tails to localize at the plasma membrane; and hydrophobicity or alkyl chain to localize at the endoplasmic reticulum. In particular embodiments, localizing cassettes are fused to a sensor molecule such as a GECI. In particular embodiments, fusion proteins of a GECI and a localizing cassette includes soma-jGCaMP8s, axon-jRGECO1a, syGCaMP5G, and soma-jGCaMP7s.

In particular embodiments, functional molecules include tag cassettes. A tag cassette includes His tag (HHHHHH; SEQ ID NO: 136), Flag tag (DYKDDDDK; SEQ ID NO: 137), Xpress tag (DLYDDDDK; SEQ ID NO: 138), Avi tag (GLNDIFEAQKIEWHE; SEQ ID NO: 139), Calmodulin tag (KRRWKKNFIAVSAANRFKKISSSGAL; SEQ ID NO: 140), Polyglutamate tag, HA tag (YPYDVPDYA; SEQ ID NO: 141), Myc tag (EQKLISEEDL; SEQ ID NO: 142), Strep tag (which refers the original STREP® tag (WRHPQFGG; SEQ ID NO: 143), STREP® tag II (WSHPQFEK SEQ ID NO: 144 (IBA Institut fur Bioanalytik, Germany); see, e.g., U.S. Pat. No. 7,981,632), Softag 1 (SLAELLNAGLGGS; SEQ ID NO: 145), Softag 3 (TQDPSRVG; SEQ ID NO: 146), and V5 tag (GKPIPNPLLGLDST; SEQ ID NO: 147). In particular embodiments, a tag cassette includes a fusion of tag cassettes such as 3XFLAG. In particular embodiments, 3XFLAG includes the sequence set forth in SEQ ID NO: 68.

Sequences are publicly-available. As examples, lactase (e.g., GenBank: EAX11622.1), lipase (e.g., GenBank: AAA60129.1), helicase (e.g., GenBank: AMD82207.1), amylase (e.g., GenBank: AAA51724.1), alpha-glucosidase (e.g., GenBank: ABI53718.1), transcription factor SP1 (e.g., UniProtKB/Swiss-Prot: P08047.3), transcription factor AP-1 (e.g., NP_002219.1), heat shock factor protein 1 (e.g., UniProtKB/Swiss-Prot: Q00613.1), CCAAT/enhancer-binding protein (C/EBP) beta isoform a (e.g., NP_005185.2), Oct-1 (e.g., UniProtKB/Swiss-Prot: P14859.2), TGFβ (e.g., GenBank: CAF02096.2), glial cell line-derived neurotrophic factor (GDNF) (e.g., NP_001177397.1), platelet-derived growth factor receptor (e.g., GenBank: AAA60049.1), epidermal growth factor receptor (e.g., GenBank: CAA25240.1), vascular endothelial growth factor receptor (e.g., GenBank: AAC16449.2), interleukin 8 receptor alpha (e.g., GenBank: AAB59436.1), caveolin (e.g., GenBank: CAA79476.1), dynamin (e.g., GenBank: AAA88025.1), clathrin heavy chain 1 isoform 1 (e.g., NP_004850.1), clathrin heavy chain 2 isoform 1 (e.g., NP_009029.3), clathrin light chain A isoform a (e.g., NP_001824.1), clathrin light chain B isoform a (e.g., NP_001825.1), ras-related protein Rab-4A isoform 1 (e.g., NP_004569.2), ras-related protein Rab-11A (e.g., UniProtKB/Swiss-Prot: P62491.3), platelet-derived growth factor (e.g., GenBank: AAA60552.1), transforming growth factor-beta3 (e.g., GenBank: AAA61161.1), nerve growth factor (e.g., GenBank: CAA37703.1), EGF (e.g., GenBank: CAA34902.2), cocaine and amphetamine regulated transcript (Chain A) (e.g., PDB: 1HY9_A), protachykinin-1 (e.g., UniProtKB-P20366), oxytocin-neurophysin 1 (e.g., UniProtKB-P01178), somatostatin (e.g., GenBank: AAH32625.1), genetically-encoded green calcium indicator NTnC (chain A) [synthetic construct] (e.g., PDB: 5MWC_A), calcium indicator TN-XXL [synthetic construct], (e.g., GenBank: ACF93133.1), BRET-based auto-luminescent calcium indicator [synthetic construct] (e.g., GenBank ADF42668.1), calcium indicator protein OeNL (Ca2+)-18u [synthetic construct], ((e.g., GenBank BBB18812.1), myosin light chain kinase, Green fluorescent protein, Calmodulin chimera (Chain A) [synthetic construct] ((e.g., PDB: 3EKJ_A), channelopsin 1 (e.g., UniProtKB-F8UVI5), channelopsin 1 (e.g., GenBank: AER58217.1), channelrhodopsin-2 ((e.g., UniProtKB-B4Y105), channel rhodopsin 2 [synthetic construct] ((e.g., GenBank: ABO64386.1), CRISPR-associated protein (Cas) (e.g., GenBank: AKG27598.1), Cas9 [synthetic construct] (e.g., GenBank: AST09977.1), CRISPR-associated endonuclease Cpf1 (e.g., UniProtKB/Swiss-Prot: U2UMQ6.1), ribonuclease 4 or ribonuclease L (e.g., UniProtKB/Swiss-Prot: Q05823.2), deoxyribonuclease II beta (e.g., GenBank: AAF76893.1), sodium channel protein type 1 subunit alpha (e.g., UniProtKB-P35498), potassium voltage-gated channel subfamily KQT member 2 (e.g., UniProtKB-O43526), and voltage-dependent L-type calcium channel subunit alpha-1C (e.g., UniProtKB-Q13936).

Additional effector elements include Cre, iCre, dgCre, FlpO, and tTA2. iCre refers to a codon-improved Cre. dgCre refers to an enhanced GFP/Cre recombinase fusion gene with an N terminal fusion of the first 159 amino acids of the Escherichia coli K-12 strain chromosomal dihydrofolate reductase gene (DHFR or folA) harboring a G67S mutation and modified to also include the R12Y/Y1001 destabilizing domain mutation. FIpO refers to a codon-optimized form of FLPe that greatly increases protein expression and FRT recombination efficiency in mouse cells. Like the Cre/LoxP system, the FLP/FRT system has been widely used for gene expression (and generating conditional knockout mice, mediated by the FLP/FRT system). tTA2 refers to tetracycline transactivator.

Exemplary expressible elements are expression products that do not include effector elements, for example, a non-functioning or defective protein. In particular embodiments, expressible elements can provide methods to study the effects of their functioning counterparts. In particular embodiments, expressible elements are non-functioning or defective based on an engineered mutation that renders them non-functioning. In these aspects, non-expressible elements are as similar in structure as possible to their functioning counterparts.

Exemplary self-cleaving peptides include the 2A peptides which lead to the production of two proteins from one mRNA. The 2A sequences are short (e.g., 20 amino acids), allowing more use in size-limited constructs. Particular examples include P2A, T2A, E2A, and F2A. In particular embodiments, the artificial expression constructs include an internal ribosome entry site (IRES) sequence. IRES allow ribosomes to initiate translation at a second internal site on a mRNA molecule, leading to production of two proteins from one mRNA.

Artificial expression constructs can encode nuclear localization proteins, such as Histone H1, Histone H2A, Histone H2B, Histone H3, Histone H4, histone-like protein HPhA, or H2B*.

Coding sequences encoding molecules (e.g., RNA, proteins) described herein can be obtained from publicly available databases and publications. Coding sequences can further include various sequence polymorphisms, mutations, and/or sequence variants wherein such alterations do not affect the function of the encoded molecule. The term “encode” or “encoding” refers to a property of sequences of nucleic acids, such as a vector, a plasmid, a gene, cDNA, mRNA, to serve as templates for synthesis of other molecules such as proteins.

The term “gene” may include not only coding sequences but also regulatory regions such as promoters, enhancers, insulators, and/or post-regulatory elements, such as termination regions. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include minBglobin (also referred to as minBGprom), CMV, minCMV, minCMV* (minCMV* is minCMV with a Sacl restriction site removed), minRho, minRho* (minRho* is minRho with a Sacl restriction site removed), SV40 immediately early promoter, the Hsp68 minimal promoter (proHSP68), and the Rous Sarcoma Virus (RSV) long-terminal repeat (LTR) promoter. Minimal promoters have no activity to drive gene expression on their own but can be activated to drive gene expression when linked to a proximal enhancer element.

In particular embodiments, expression constructs are provided within vectors. The term vector refers to a nucleic acid molecule capable of transferring or transporting another nucleic acid molecule, such as an expression construct. The transferred nucleic acid is generally linked to, e.g., inserted into, the vector nucleic acid molecule. A vector may include sequences that direct autonomous replication in a cell or may include sequences that permit integration into host cell DNA. Useful vectors include, for example, plasmids (e.g., DNA plasmids or RNA plasmids), transposons, cosmids, bacterial artificial chromosomes, and viral vectors.

Viral vector is widely used to refer to a nucleic acid molecule that includes virus-derived components that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Adenovirus vectors refer to those constructs containing adenovirus sequences sufficient to (a) support packaging of an artificial expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In particular embodiments, adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector. As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in targeted cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MOMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In particular embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In particular embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In particular embodiments, viral vectors include a TAR element. The term “TAR” refers to the “trans-activation response” genetic element located in the R region of lentiviral LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and U5 regions. The R region plays a role during reverse transcription in permitting the transfer of nascent DNA from one end of the genome to the other.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al., 1999, J. Virol., 73:2886); the posttranscriptional regulatory element present in hepatitis B virus (HPRE) (Smith et al., Nucleic Acids Res. 26 (21): 4818-4827, 1998); and the like (Liu et al., 1995, Genes Dev., 9:1766). In particular embodiments, vectors include a posttranscriptional regulatory element such as a WPRE or HPRE. In particular embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In particular embodiments, vectors include a polyadenylation signal 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA, hGHpA, or SV40 pA. In particular embodiments, a preferred embodiment of an expression construct includes a terminator element. These elements can serve to enhance transcript levels and to minimize read through from the construct into other plasmid sequences.

In particular embodiments, a viral vector further includes one or more insulator elements. Insulators elements may contribute to protecting viral vector-expressed sequences, e.g., effector elements or expressible elements, from integration site effects, which may be mediated by cis-acting elements present in genomic DNA and lead to deregulated expression of transferred sequences (i.e., position effect; see, e.g., Burgess-Beusse et al., PNAS., USA, 99:16433, 2002; and Zhan et al., Hum. Genet., 109:471, 2001). In particular embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken β-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33:198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

Particular embodiments of vectors disclosed herein include:

| Expression | |
|---|---|
| Construct | |
| Name | Features |
| CN3062 | pAAV: eHGT_409h-minBglobin-SYFP2-WPRE3-BGHpA |
| CN3063 | pAAV: eHGT_410h-minBglobin-SYFP2-WPRE3-BGHpA |
| CN2979 | rAAV: eHGT_361h-minBglobin-SYFP2-WPRE3-BGHpA |
| CN2498 | rAAV: eHGT_395m-minBglobin-SYFP2-WPRE3-BGHpA |
| CN2677 | rAAV: eHGT_401h-minBglobin-SYFP2-WPRE3-BGHpA |
| CN2505 | rAAV: eHGT_391h-minBglobin-SYFP2-WPRE3-BGHpA |
| CN2499 | rAAV: eHGT_403h-minBglobin-SYFP2-WPRE3-BGHpA |
| CN4004 | rAAV: 3xCore_eHGT_641m-minBglobin-SYFP2-WPRE3-BGHpA |
| CN3575 | rAAV: 3xcore2_eHGT_400m-minBglobin-SYFP2-WPRE3-BGHpA |
| CN4254 | rAAV: 3xcore2_eHGT_401h-minBglobin-SYFP2-WPRE3-BGHpA |
| CN4255 | rAAV: 3xcore2_eHGT_409h-minBglobin-SYFP2-WPRE3-BGHpA |
| CN4256 | rAAV: 3xcore2_eHGT_410h-minBglobin-SYFP2-WPRE3-BGHpA |
| CN2806 | rAAV: eHGT_331h-minBglobin-SYFP2-WPRE3-BGHpA |
| CN2807 | rAAV: eHGT_332h-minBglobin-SYFP2-WPRE3-BGHpA |
| CN2808 | rAAV: eHGT_333h-minBglobin-SYFP2-WPRE3-BGHpA |
| CN2809 | rAAV: eHGT_335h-minBglobin-SYFP2-WPRE3-BGHpA |
| CN2812 | rAAV: eHGT_334h-minBglobin-SYFP2-WPRE3-BGHpA |
| CN2813 | rAAV: eHGT_360h-minBglobin-SYFP2-WPRE3-BGHpA |
| CN2678 | rAAV: eHGT_562h-minBglobin-SYFP2-WPRE3-BGHpA |
| CN2679 | rAAV: eHGT_563h-minBglobin-SYFP2-WPRE3-BGHpA |
| CN2680 | rAAV: eHGT_564h-minBglobin-SYFP2-WPRE3-BGHpA |
| CN2681 | rAAV: eHGT_565h-minBglobin-SYFP2-WPRE3-BGHpA |
| CN2833 | rAAV: eHGT_566m-minBglobin-SYFP2-WPRE3-BGHpA |
| CN2834 | rAAV: eHGT_567m-minBglobin-SYFP2-WPRE3-BGHpA |
| CN2835 | rAAV: eHGT_568m-minBglobin-SYFP2-WPRE3-BGHpA |
| CN2836 | rAAV: eHGT_569m-minBglobin-SYFP2-WPRE3-BGHpA |
| CN2627 | rAAV: eHGT_734m-minBglobin-SYFP2-WPRE3-BGHpA |
| CN2675 | rAAV: eHGT_312h-minBglobin-SYFP2-WPRE3-BGHpA |
| CN2344 | rAAV: eHGT_313h-minBglobin-SYFP2-WPRE3-BGHpA |
| CN3060 | pAAV: eHGT_377m-minBglobin-SYFP2-WPRE3-BGHpA |
| CN3061 | pAAV: eHGT_389m-minBglobin-SYFP2-WPRE3-BGHpA |
| CN3098 | pAAV: eHGT_380h-minBglobin-SYFP2-WPRE3-BGHpA |
| CN2493 | rAAV: eHGT_374h-minBglobin-SYFP2-WPRE3-BGHpA |
| CN3568 | rAAV: 3xcore1_eHGT_387m-minBglobin-SYFP2-WPRE3-BGHpA |
| CN2495 | rAAV: eHGT_385h-minBglobin-SYFP2-WPRE3-BGHpA |
| CN2496 | rAAV: eHGT_386h-minBglobin-SYFP2-WPRE3-BGHpA |
| CN2827 | rAAV: eHGT_378m-minBglobin-SYFP2-WPRE3-BGHpA |
| CN2846 | rAAV: eHGT_642m-minBglobin-SYFP2-WPRE3-BGHpA |
| CN3572 | rAAV: 3xcore2_eHGT_390h-minBglobin-SYFP2-WPRE3-BGHpA |
| CN4253 | rAAV: 3xcore2_eHGT_380h-minBglobin-SYFP2-WPRE3-BGHpA |
| CN4257 | rAAV: 3xcore2_eHGT_374m-minBglobin-SYFP2-WPRE3-BGHpA |
| CN2410 | rAAV: eHGT_603m-minBglobin-SYFP2-WPRE3-BGHpA |
| CN2345 | rAAV: eHGT_315h-minBglobin-SYFP2-WPRE3-BGHpA |
| CN2346 | rAAV: eHGT_316h-minBglobin-SYFP2-WPRE3-BGHpA |
| CN4268 | rAAV: 3xcore2_eHGT_375m-minBglobin-SYFP2-WPRE3-BGHpA |
| CN4522 | pAAV: TeIN-LITR-eHGT_358h-minBG-SYFP2-P2A-3XFLAG-10aa-H2B*- |
| | WPRE3-BGHpA-RITR-TeIN |
| CN4523 | pAAV: TeIN-LITR-eHGT_363h-minBG-SYFP2-P2A-3XFLAG-10aa-H2B*- |
| | WPRE3-BGHpA-RITR-TeIN |

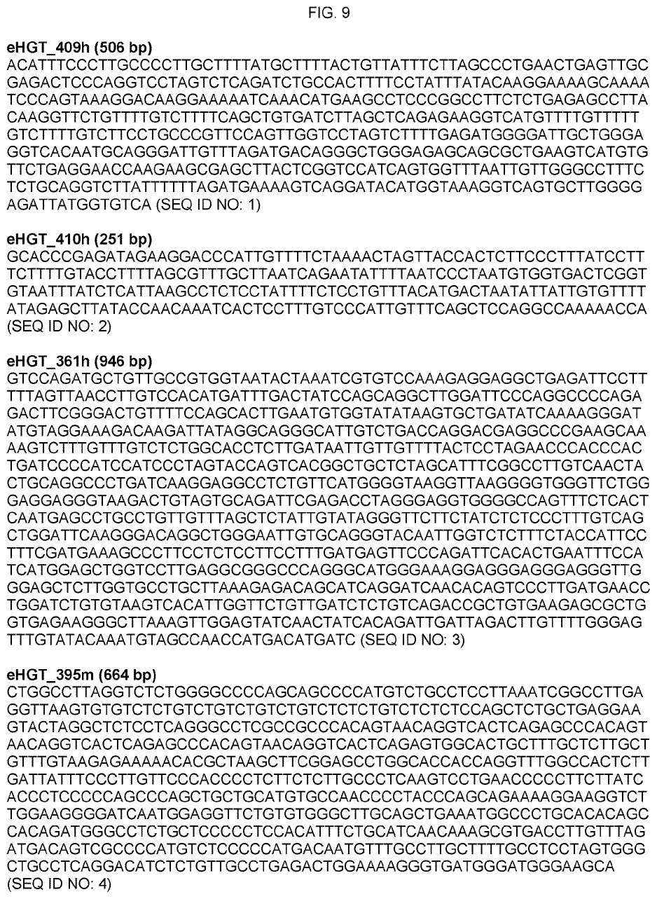

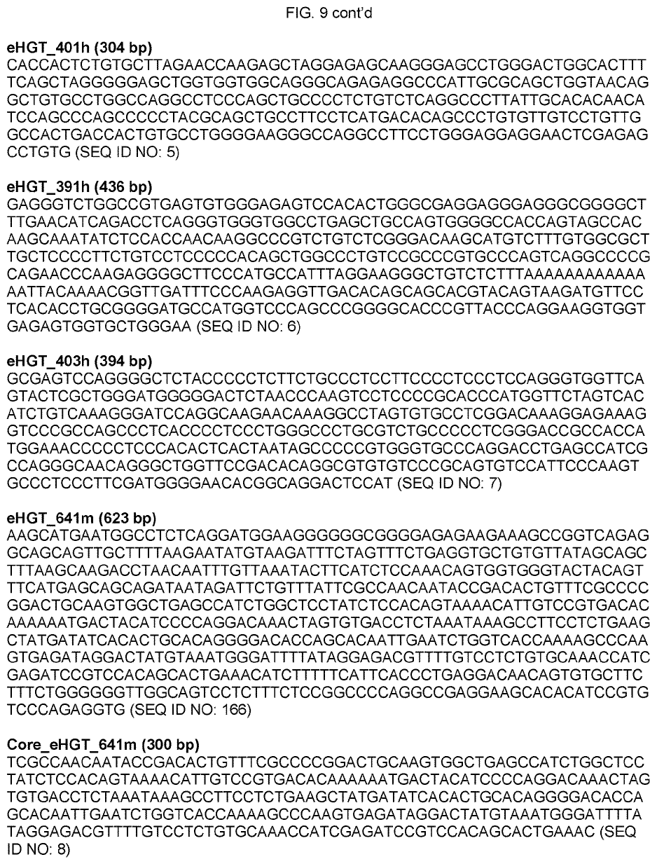

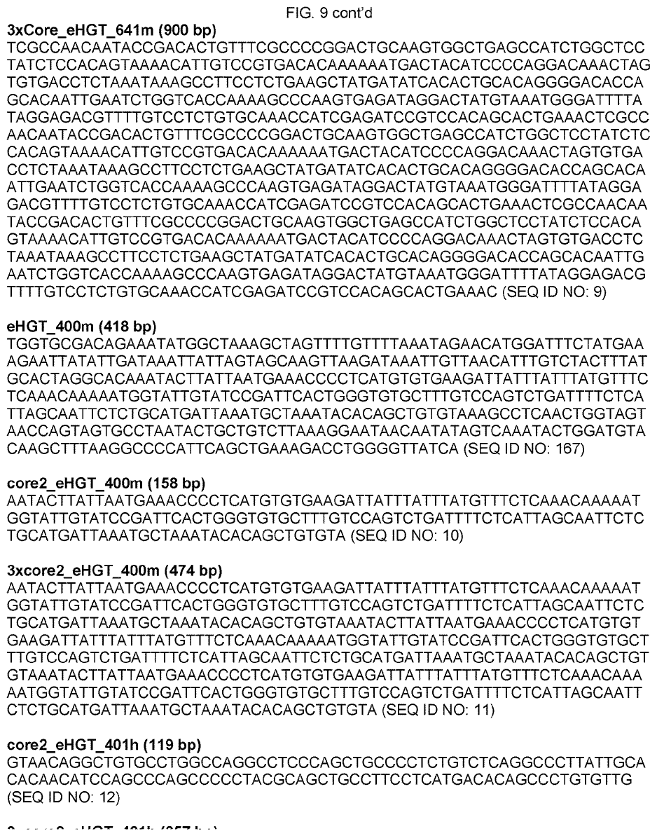

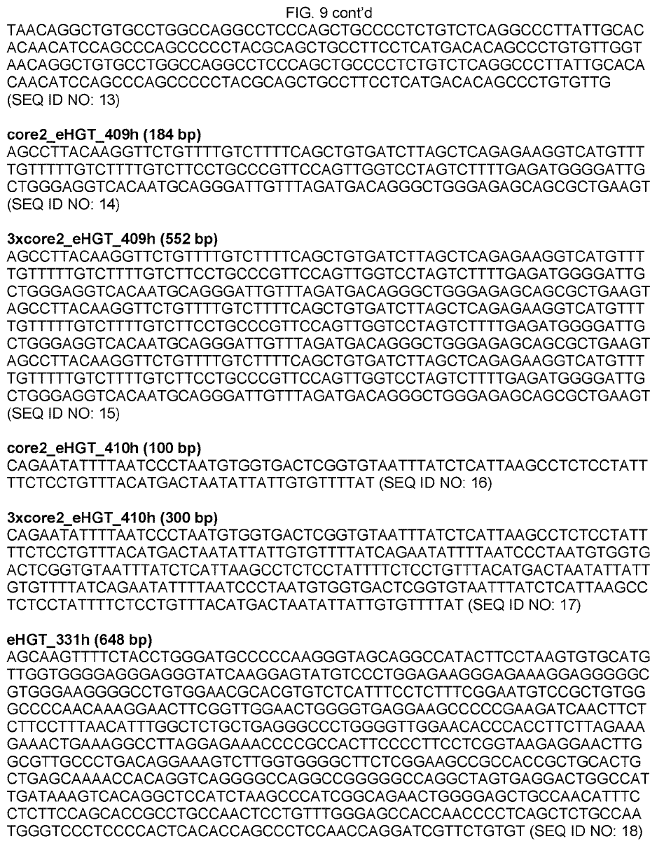

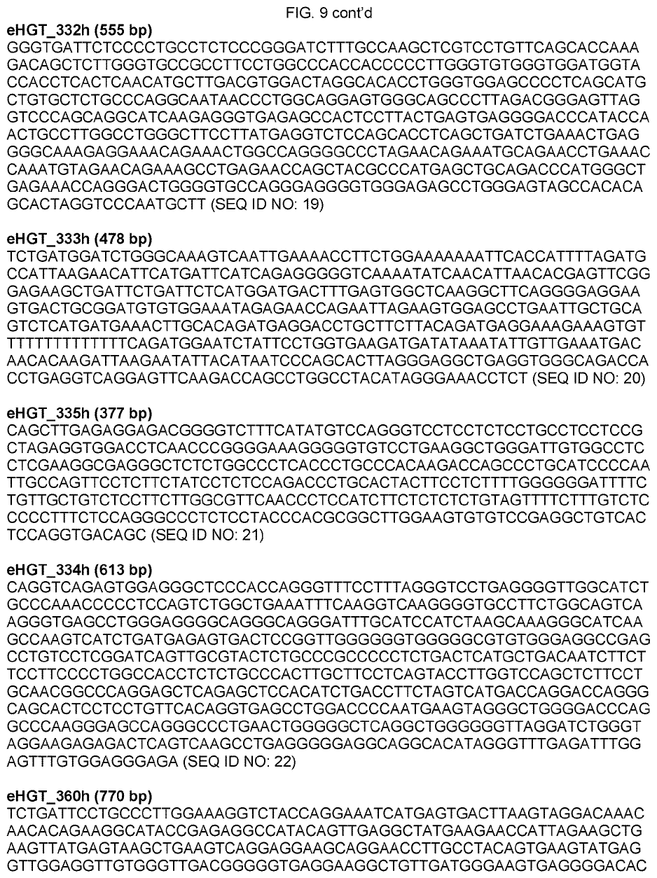

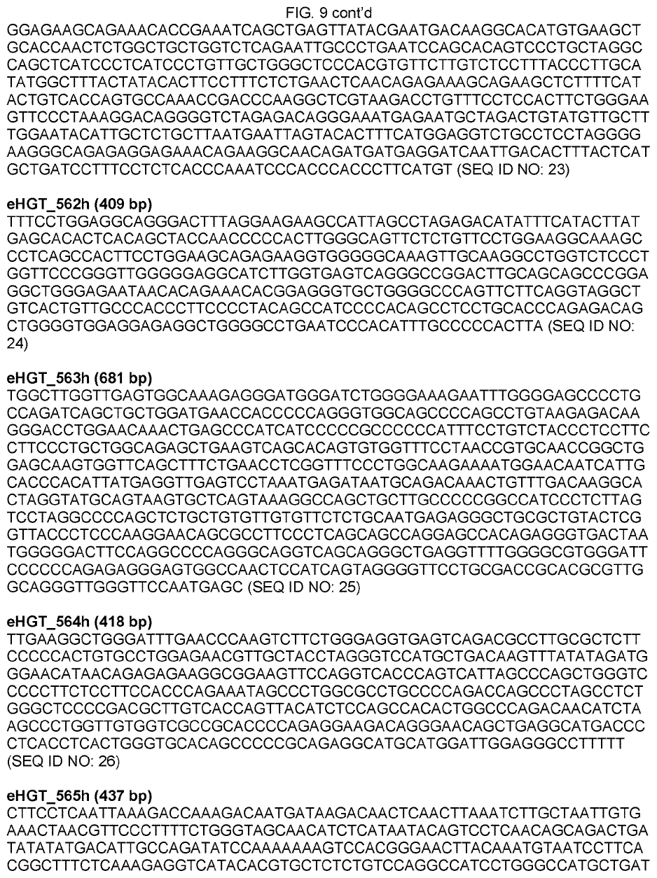

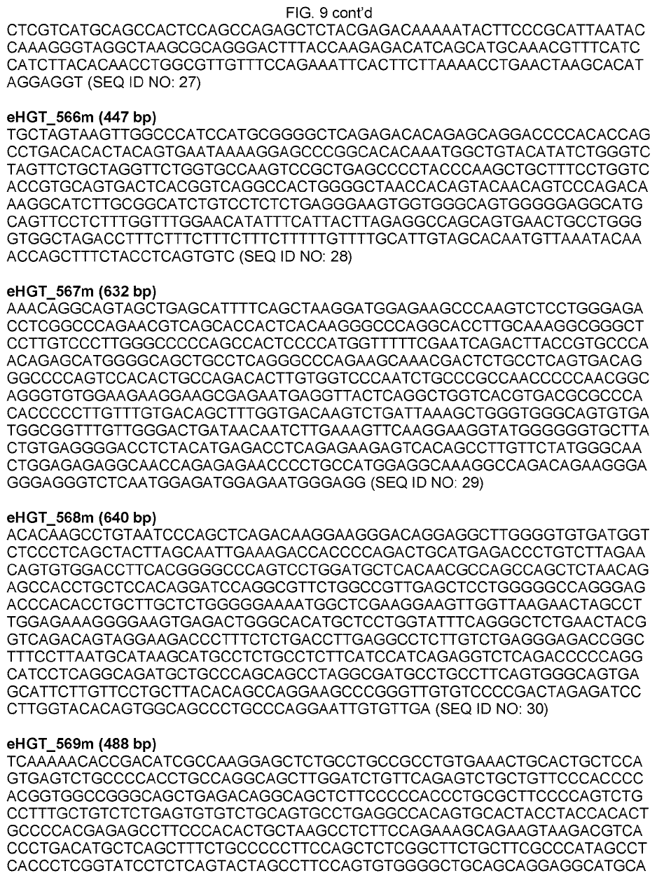

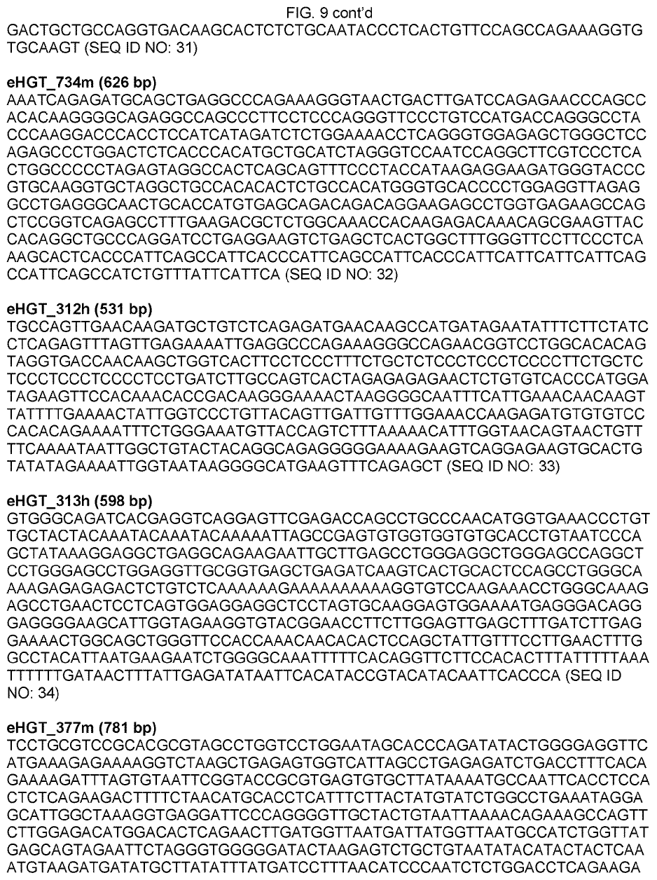

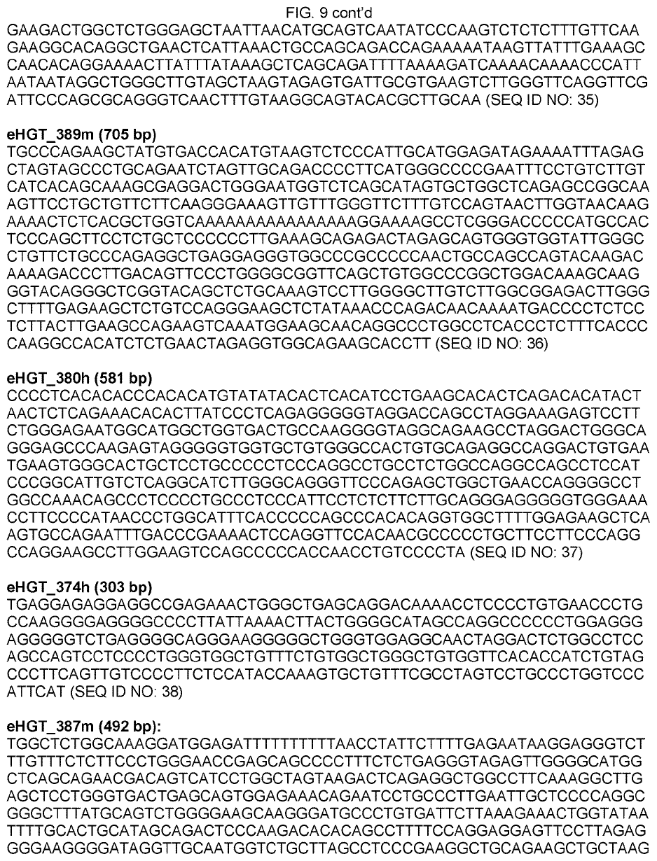

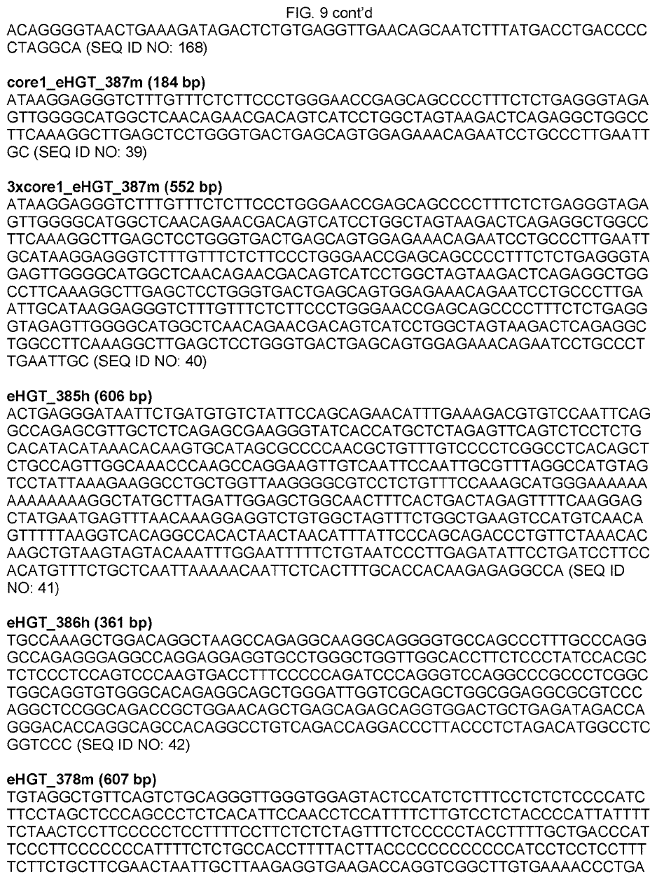

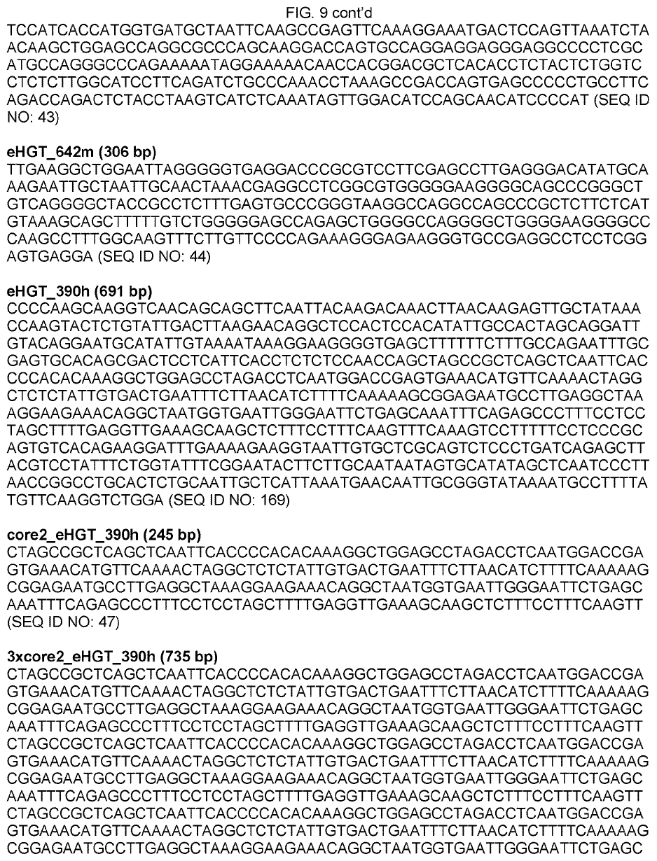

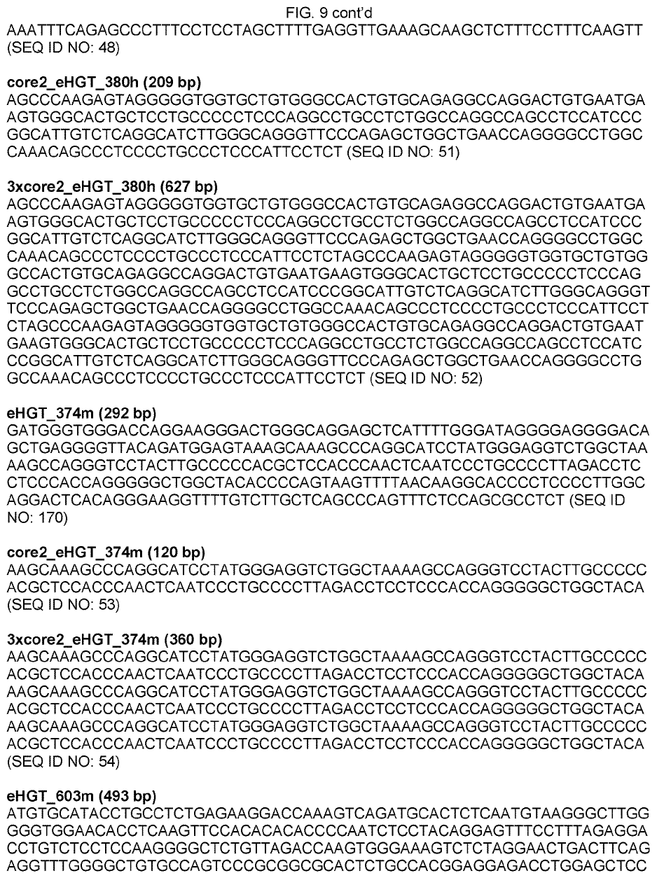

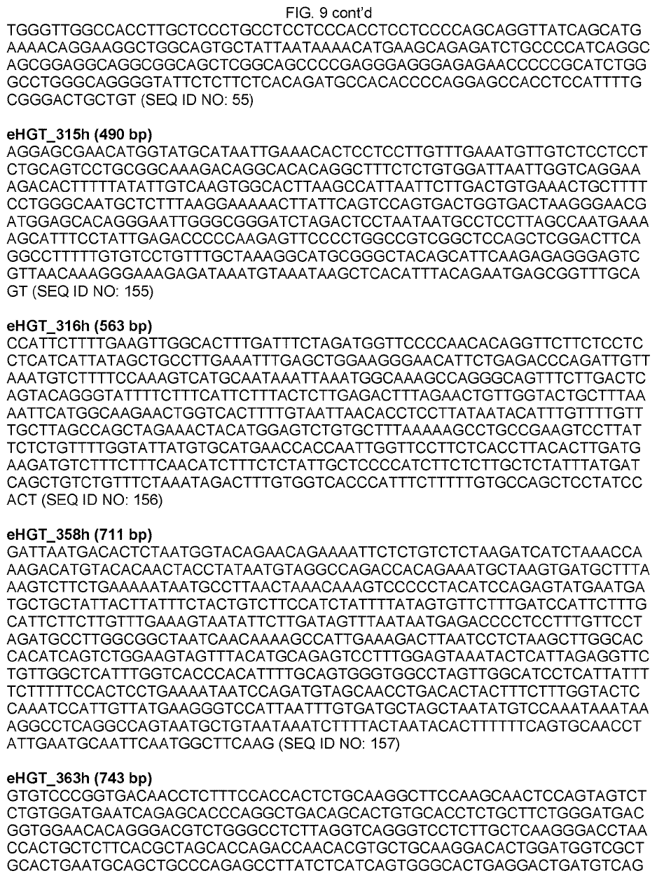

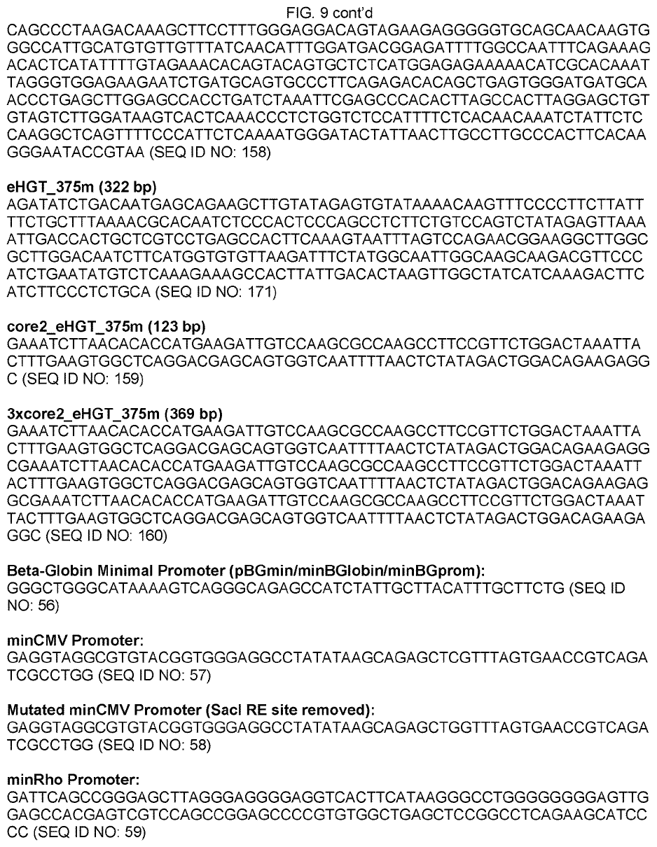

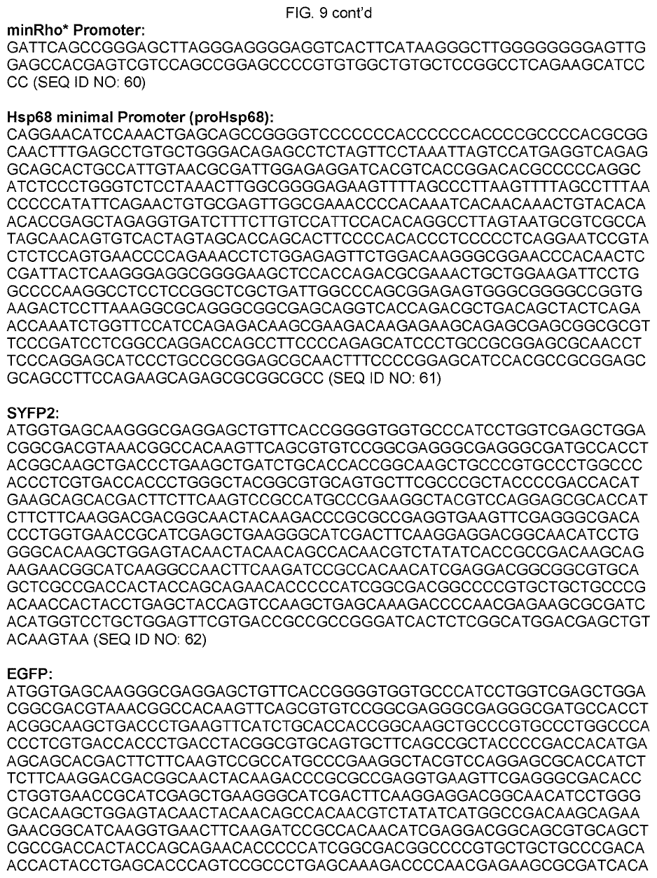

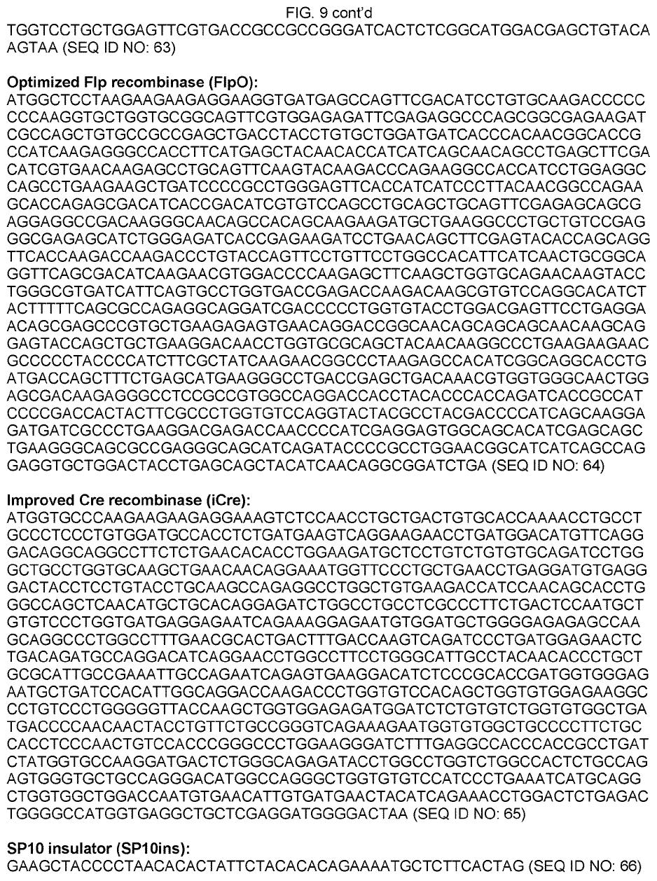

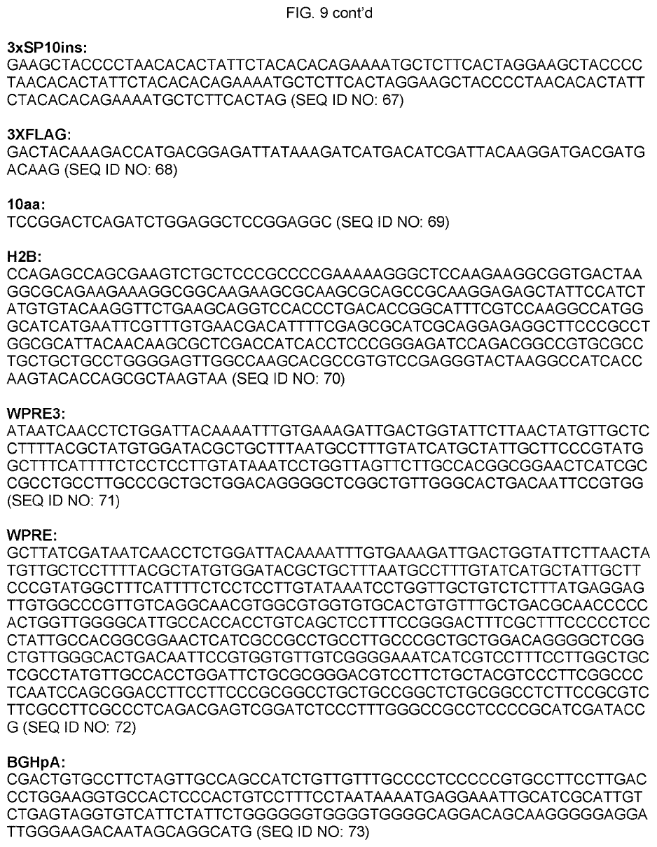

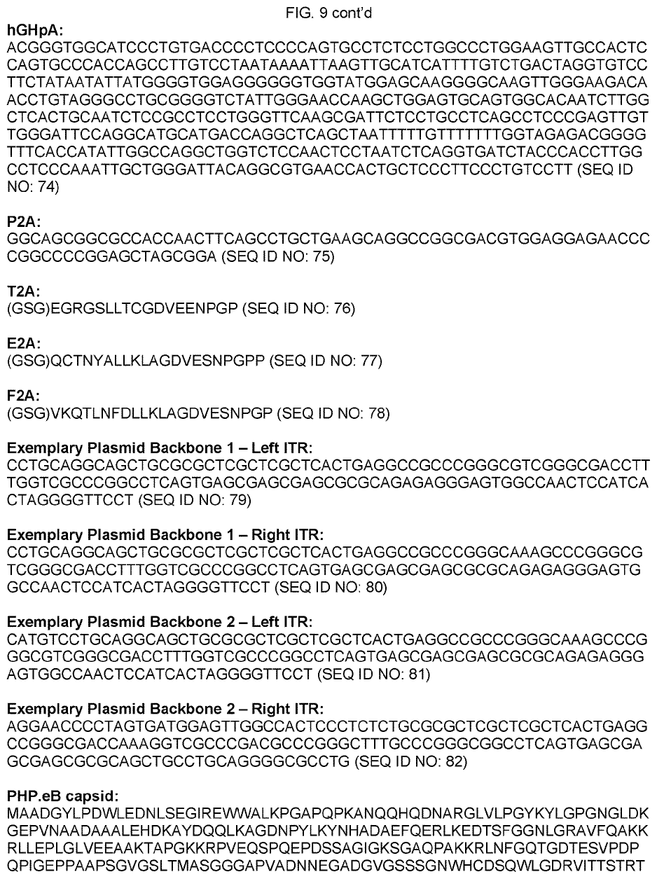

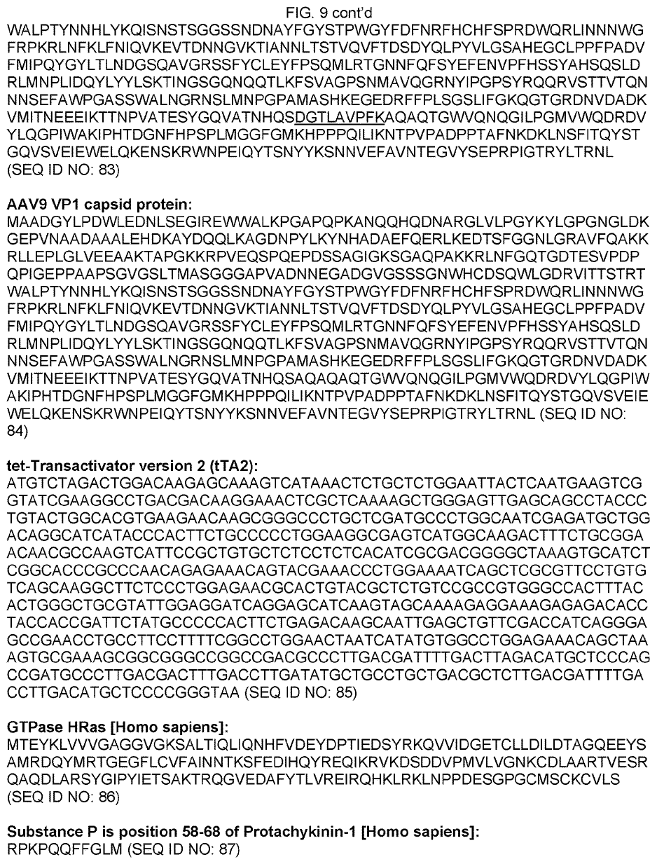

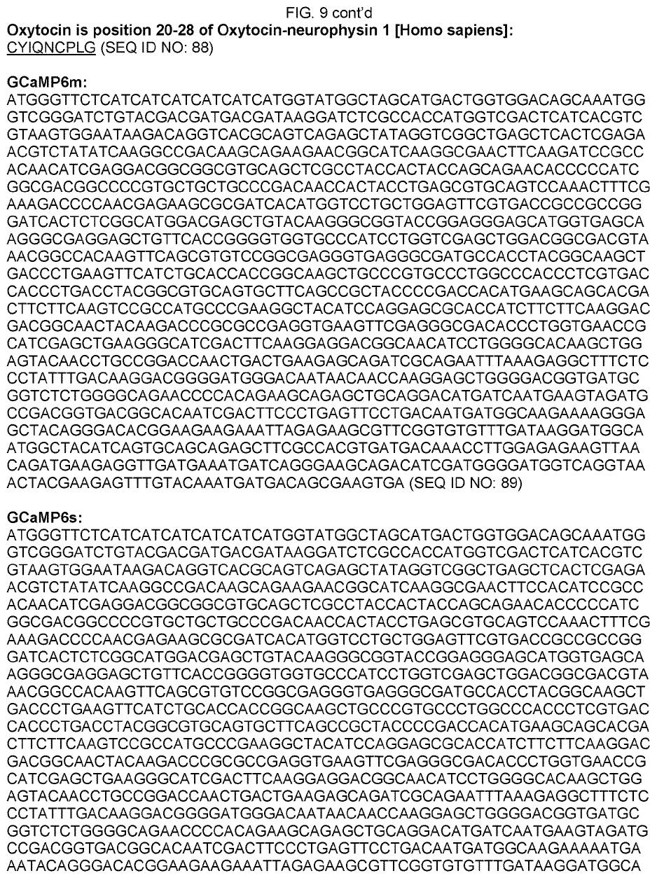

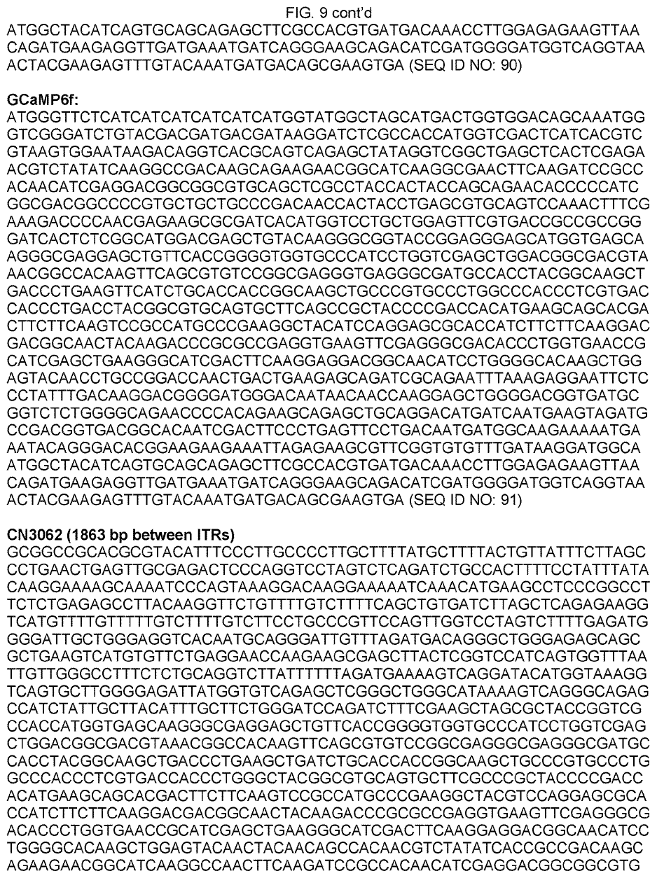

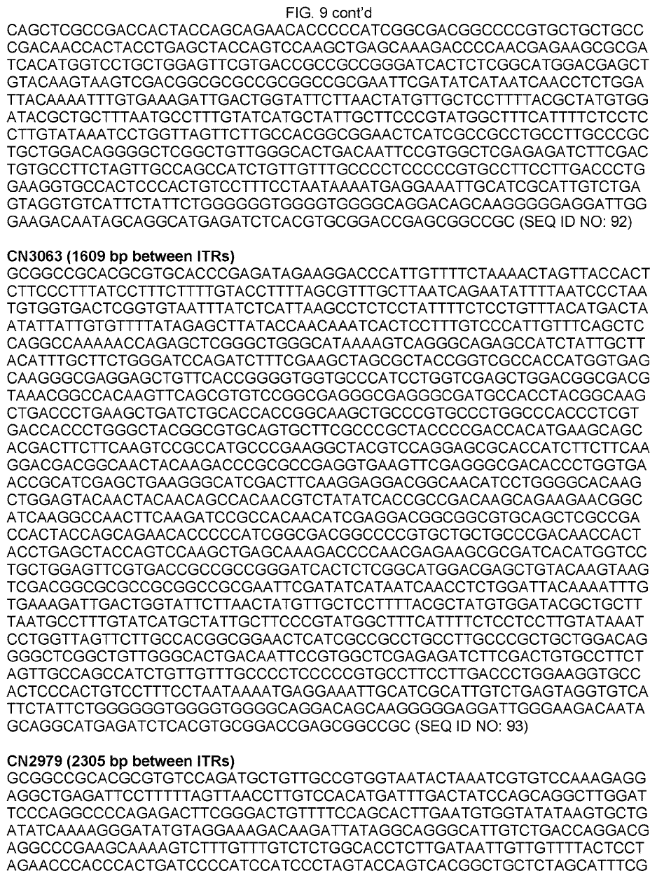

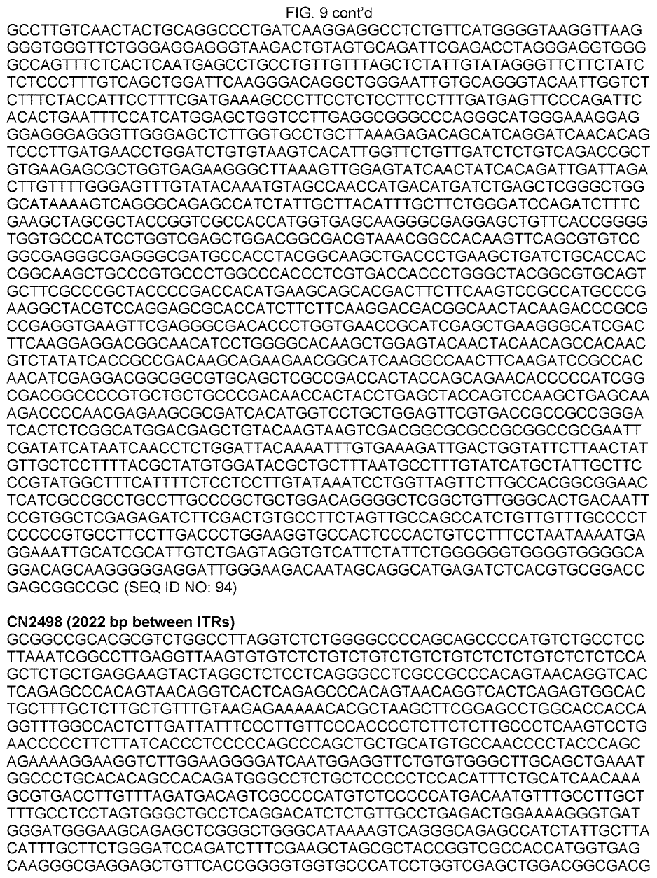

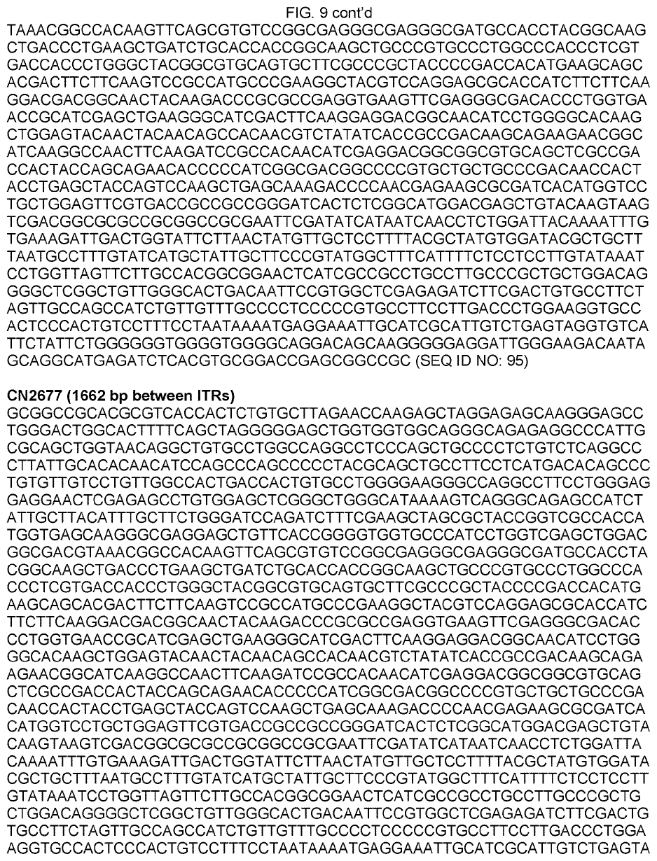

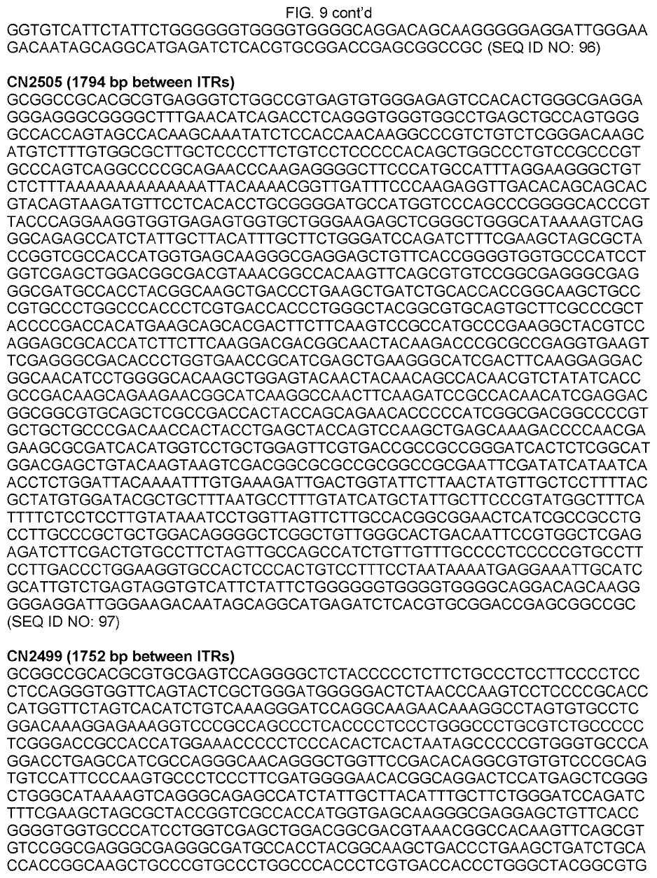

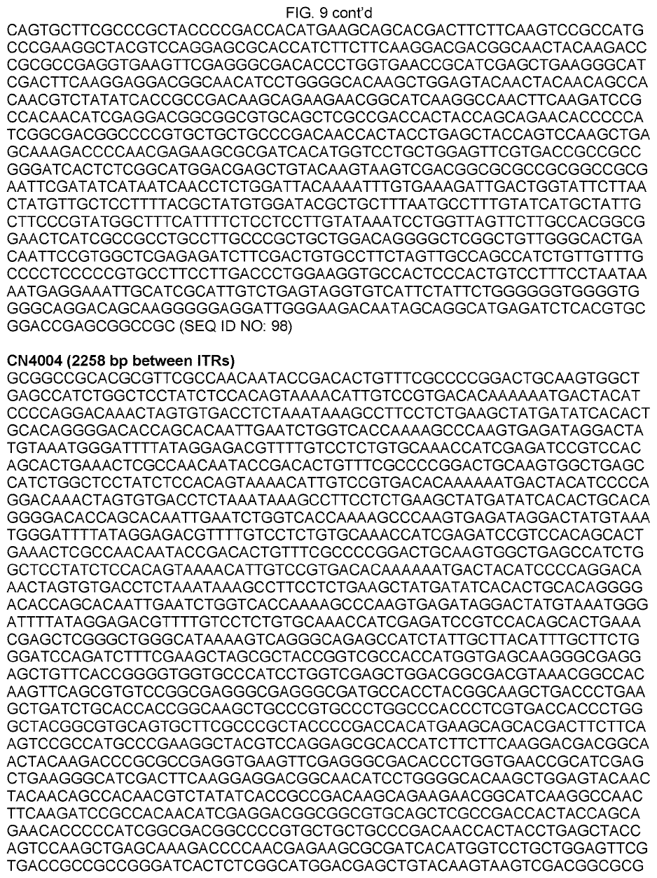

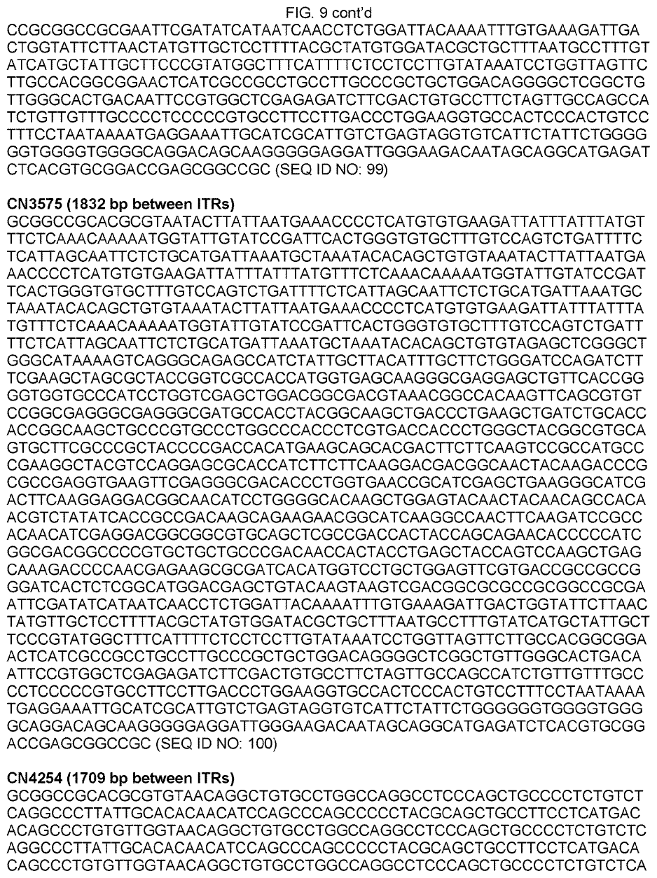

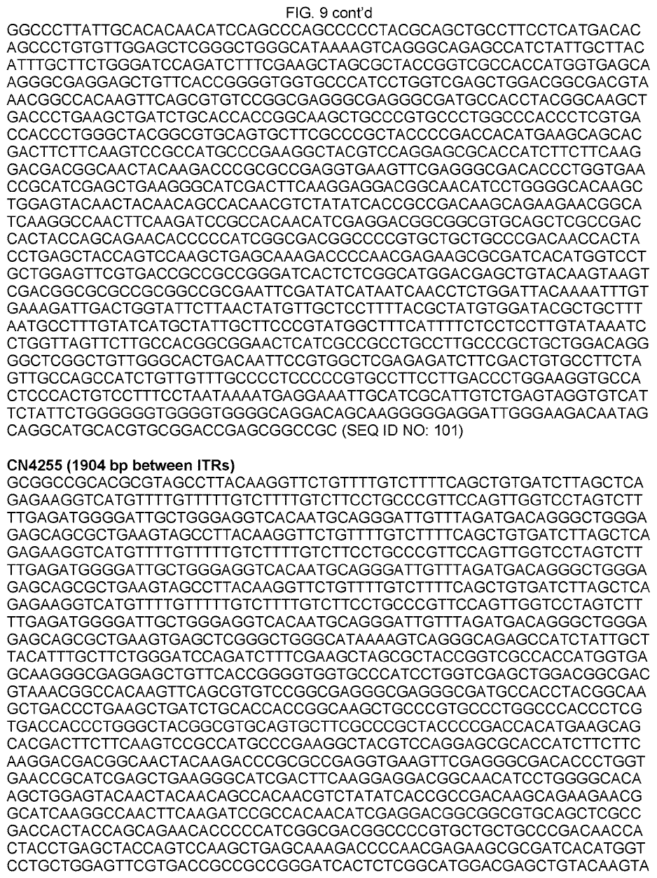

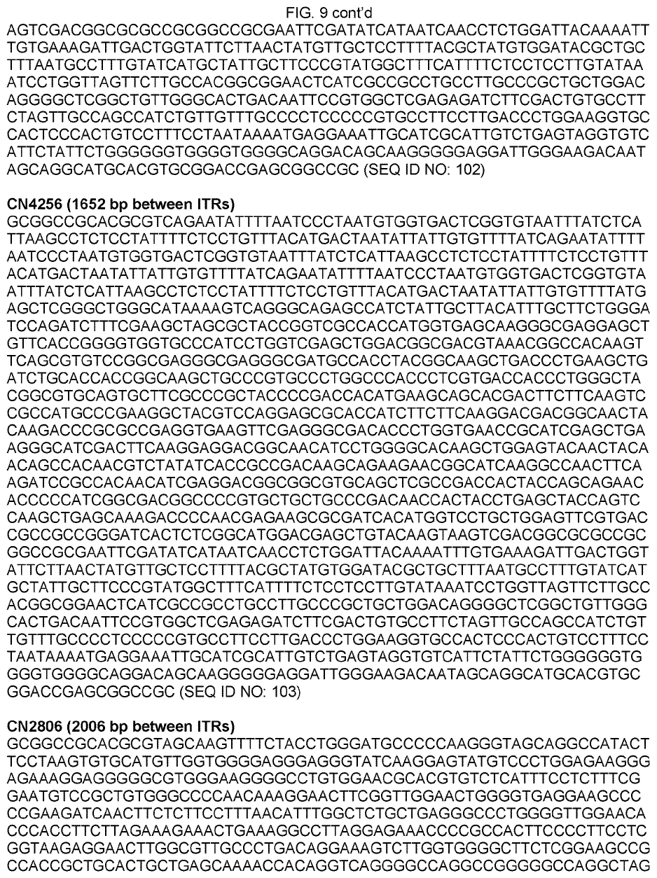

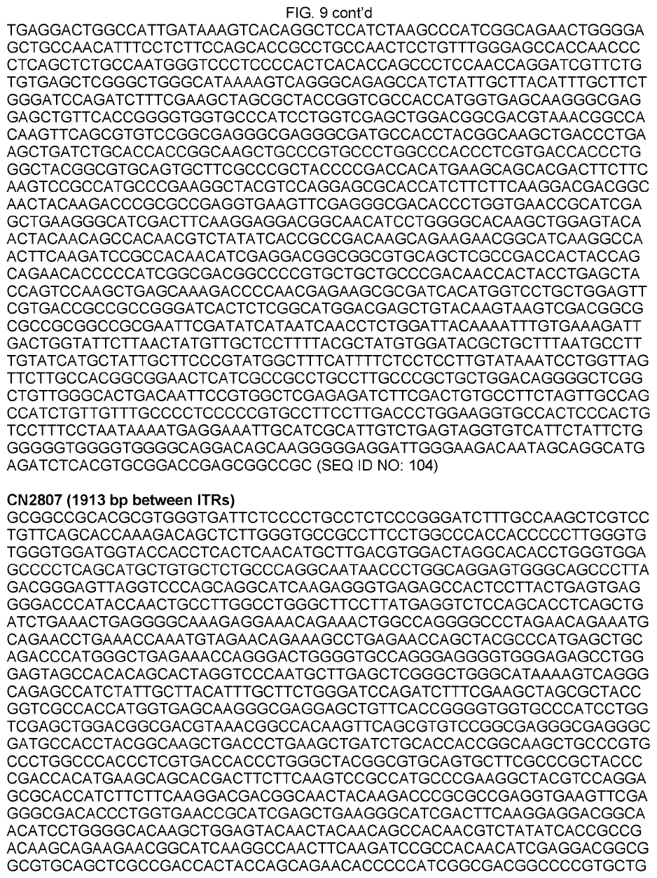

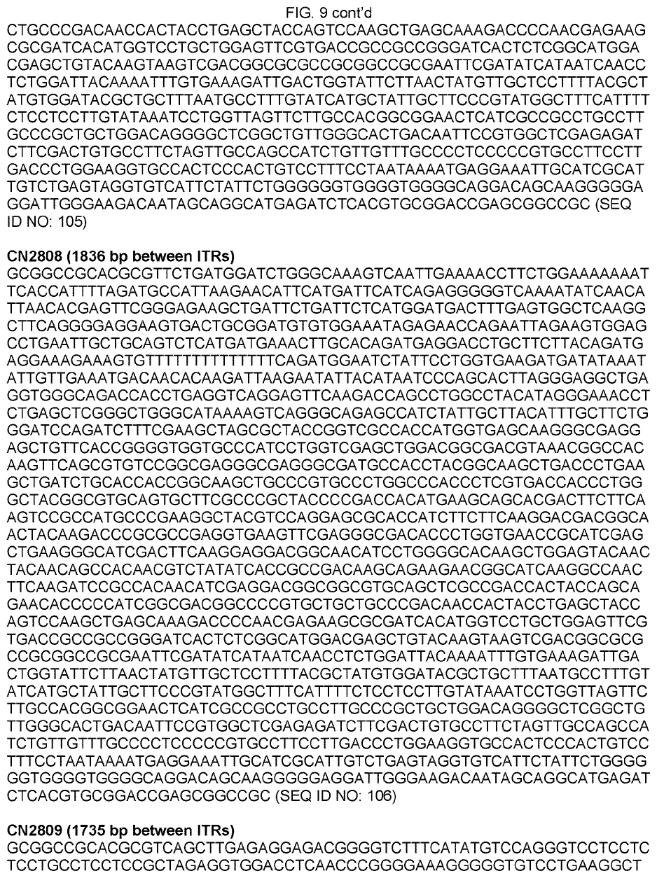

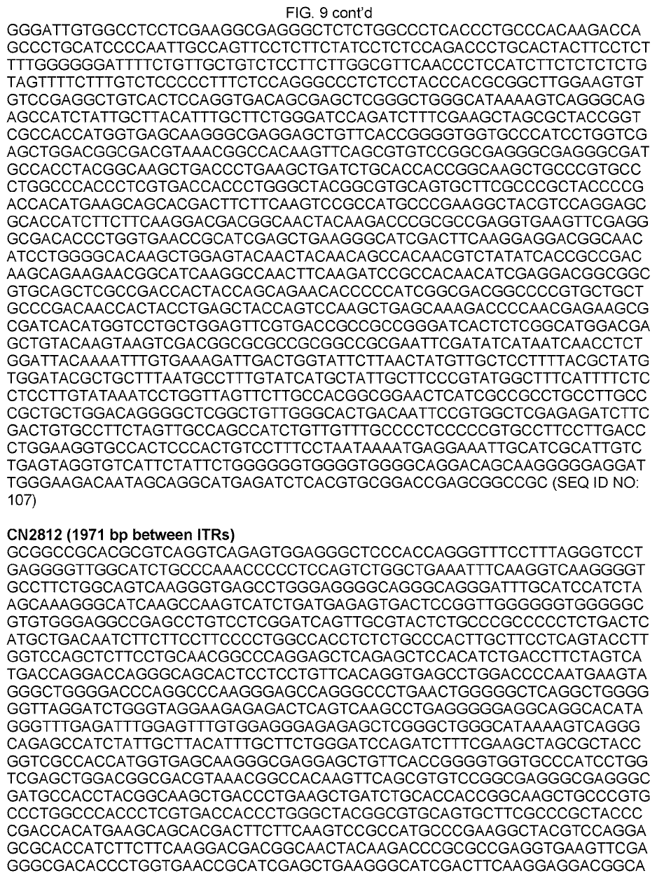

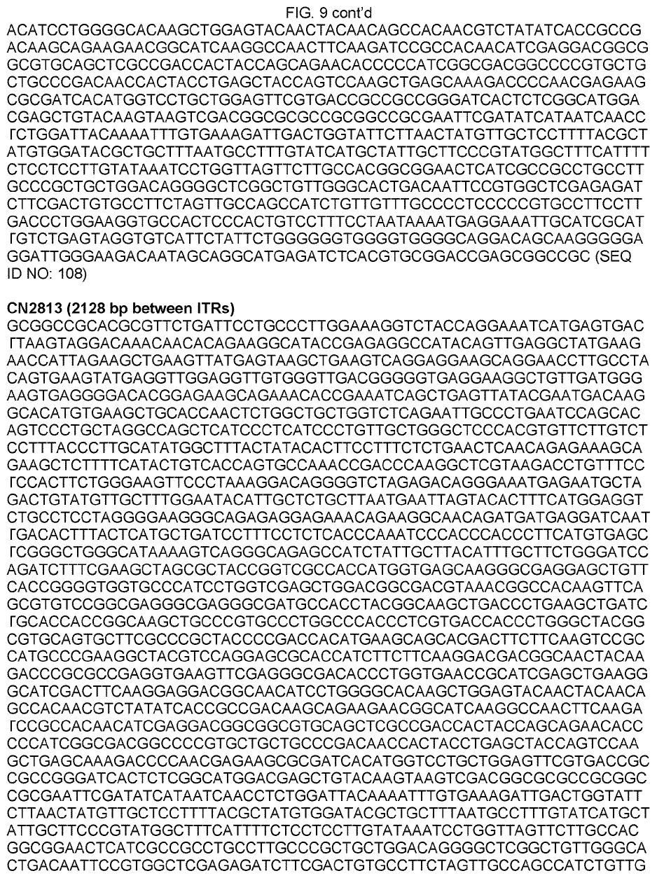

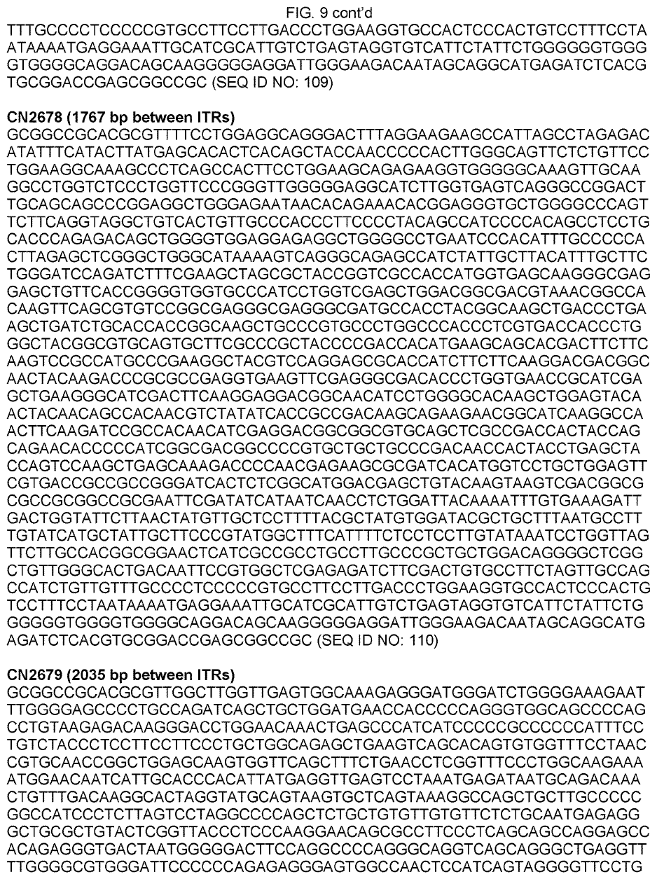

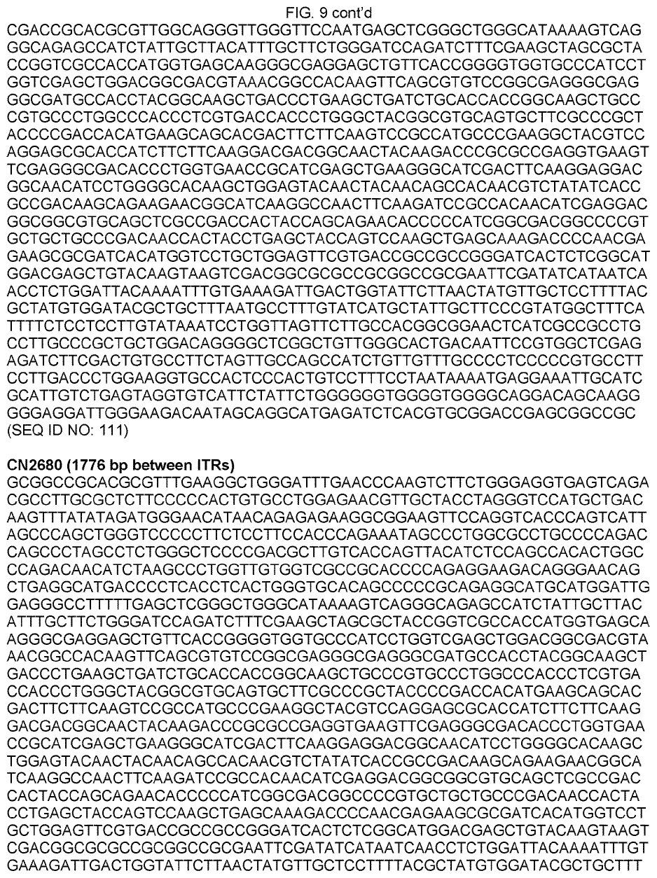

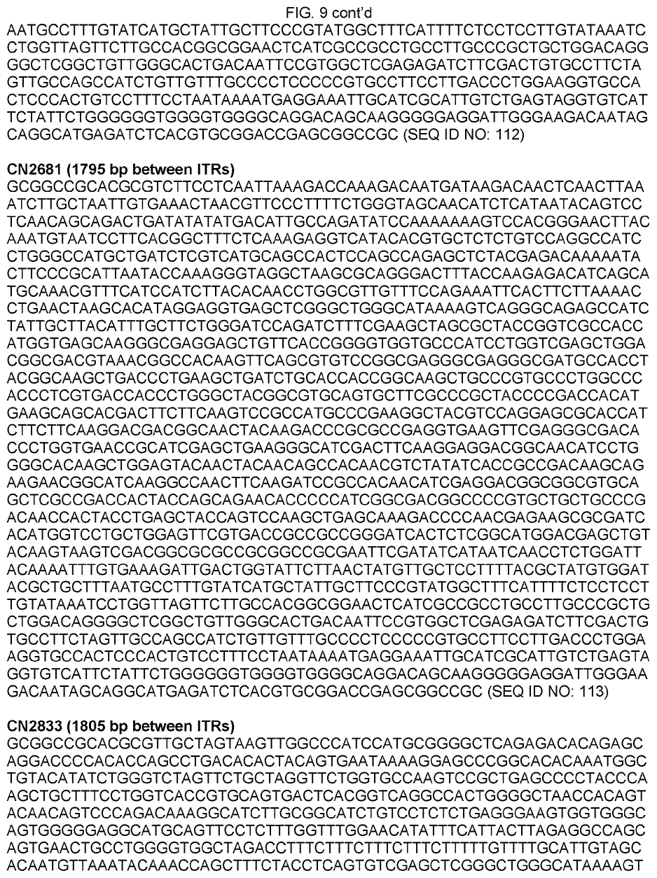

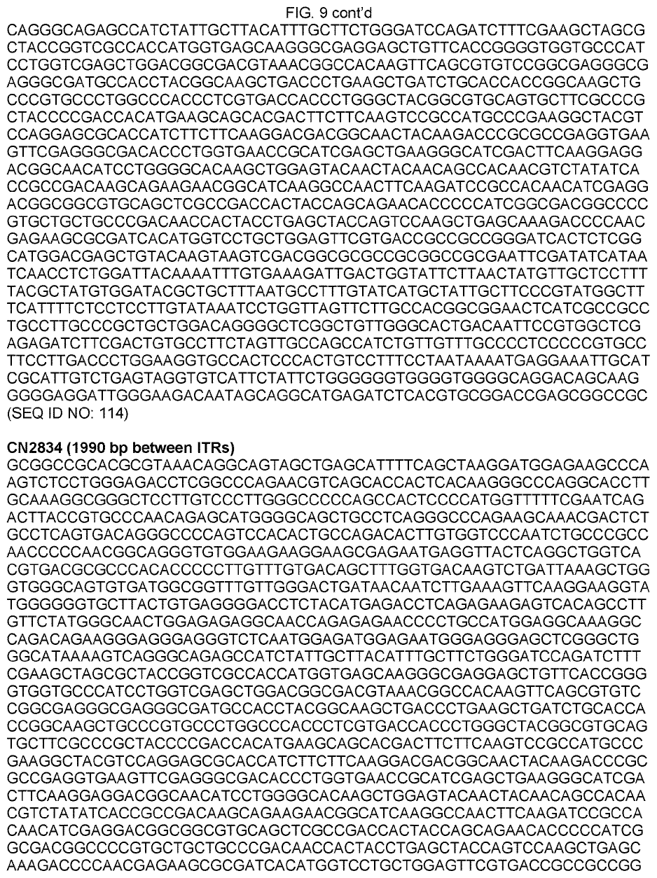

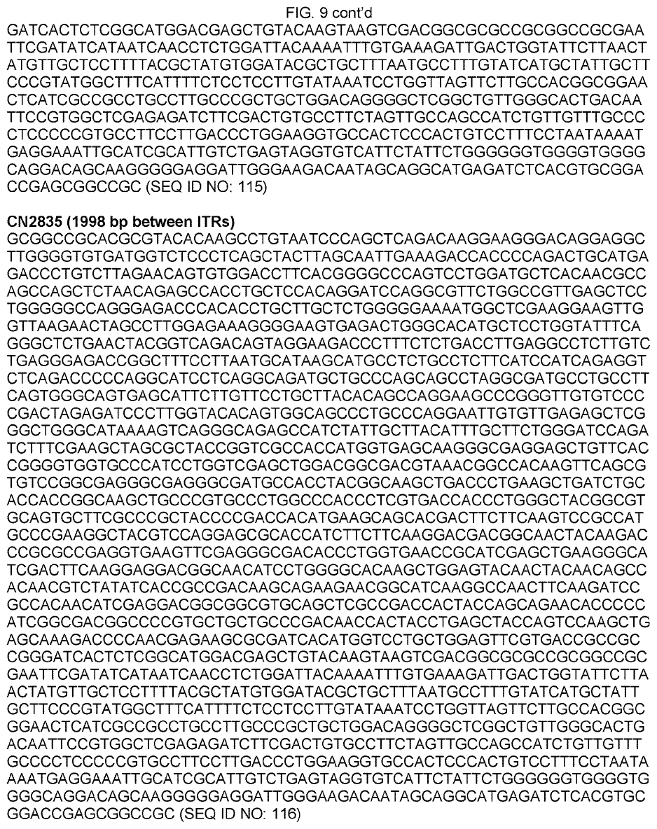

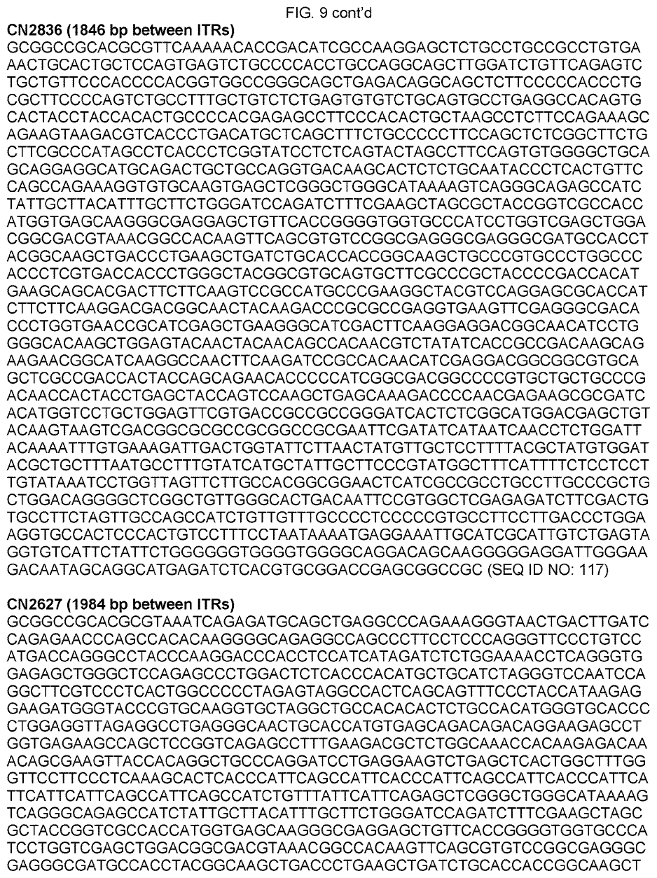

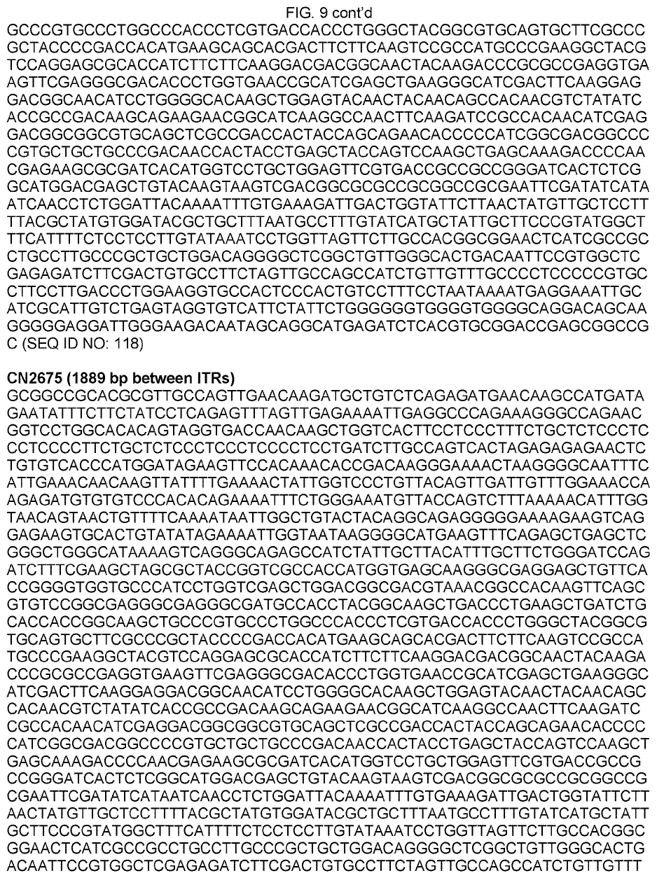

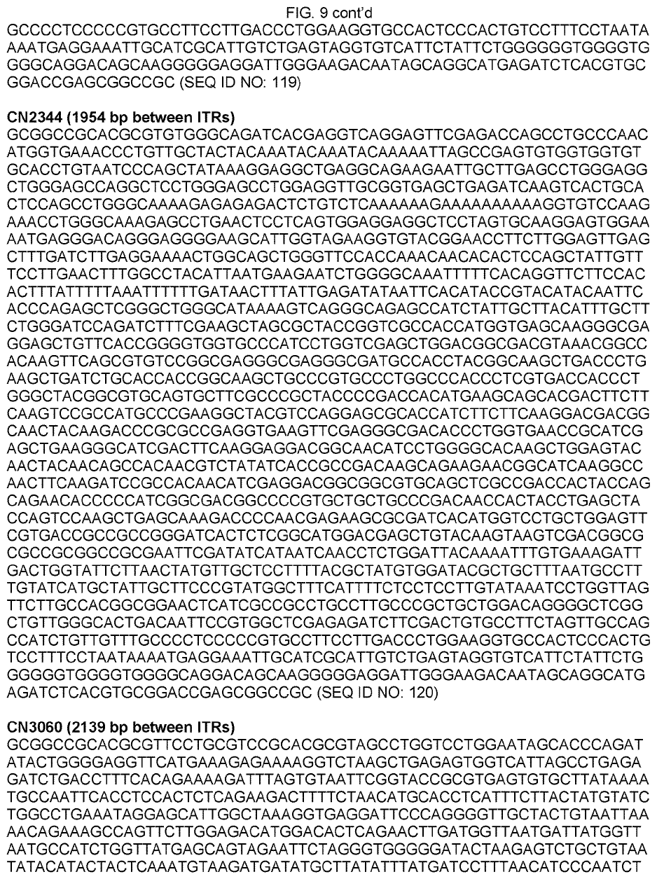

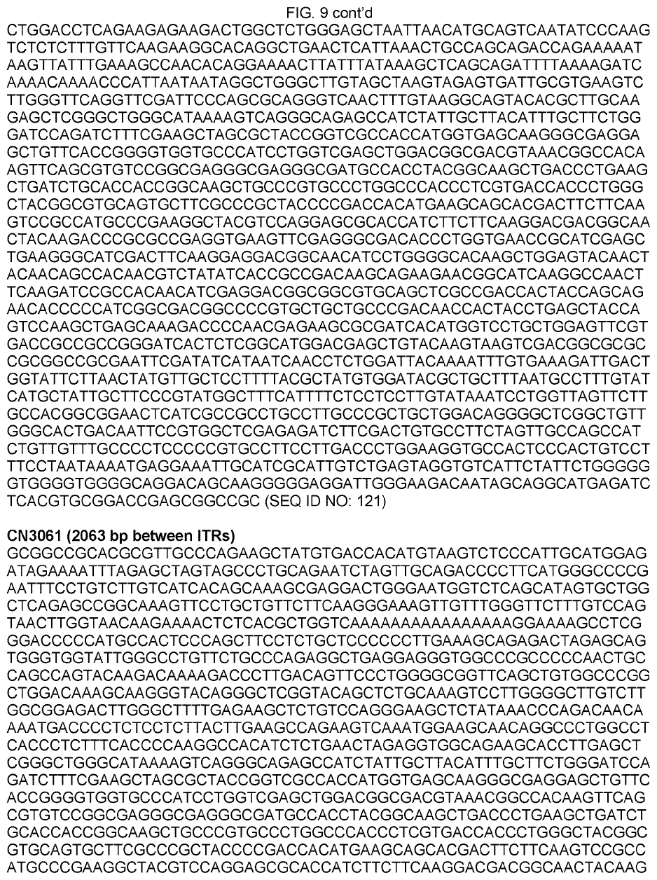

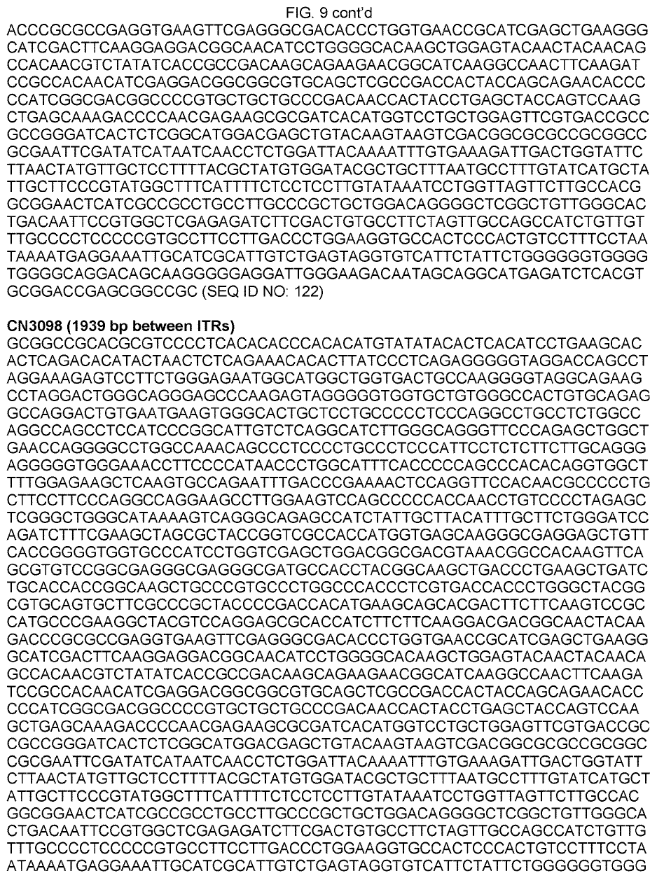

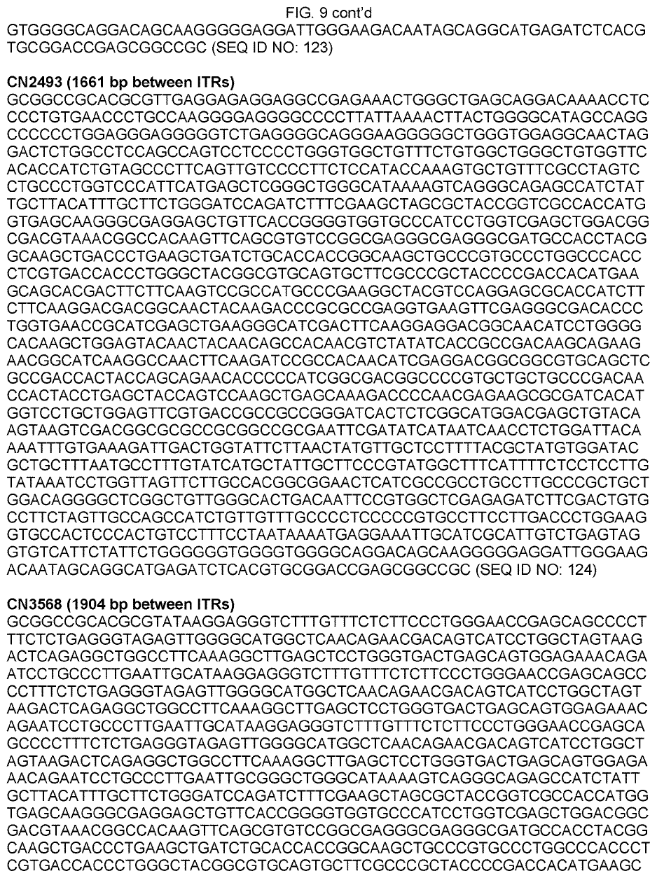

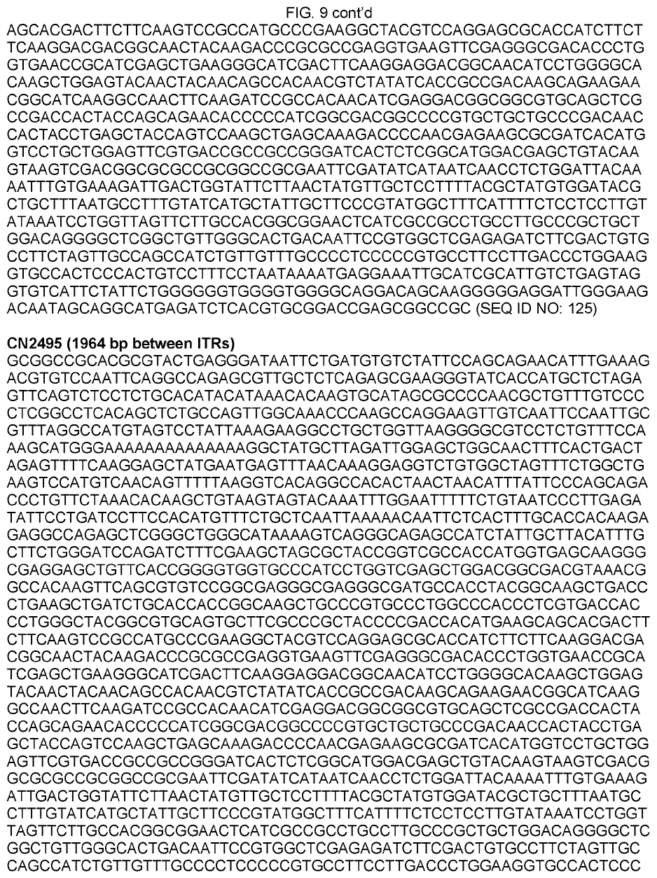

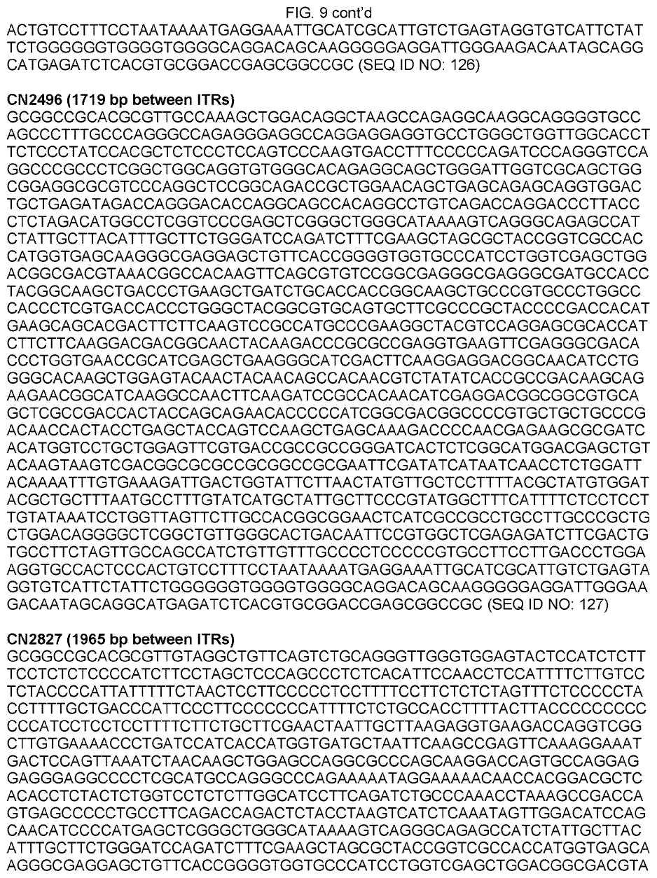

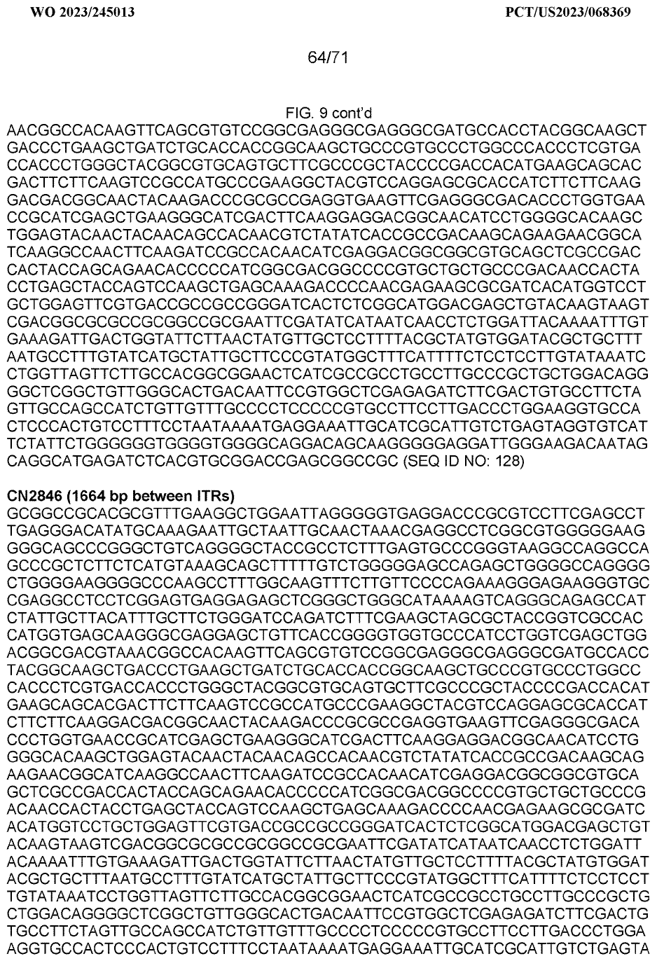

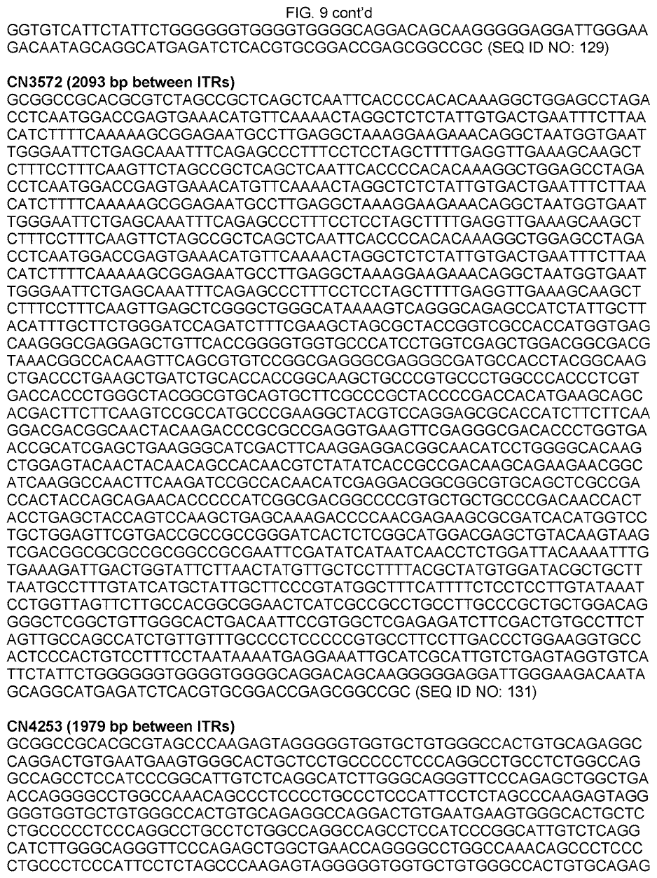

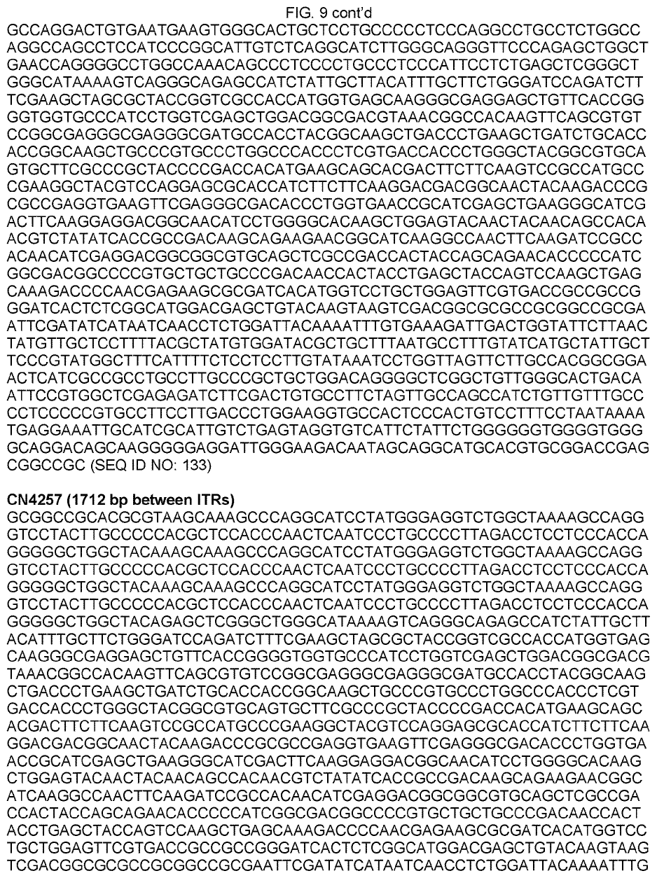

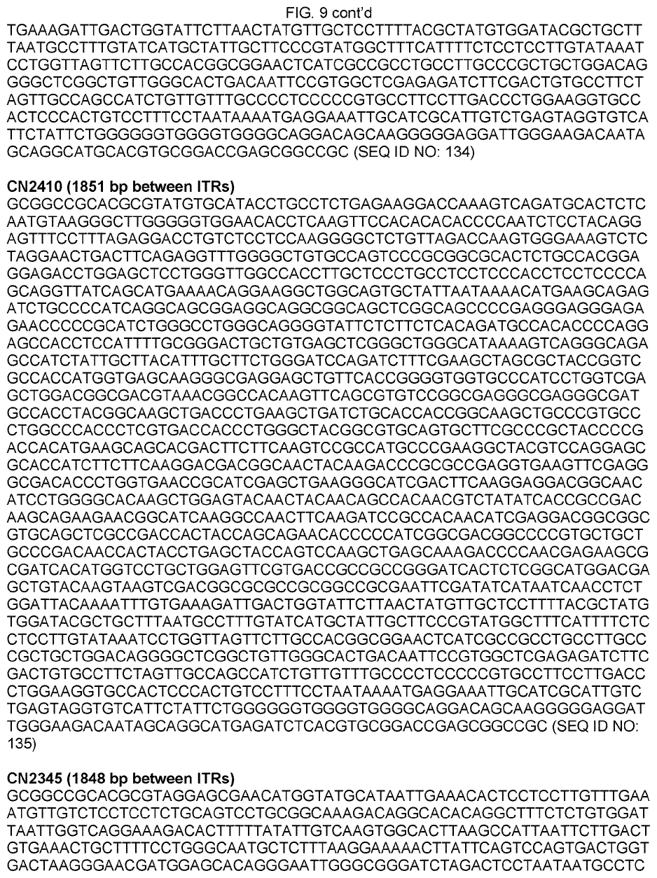

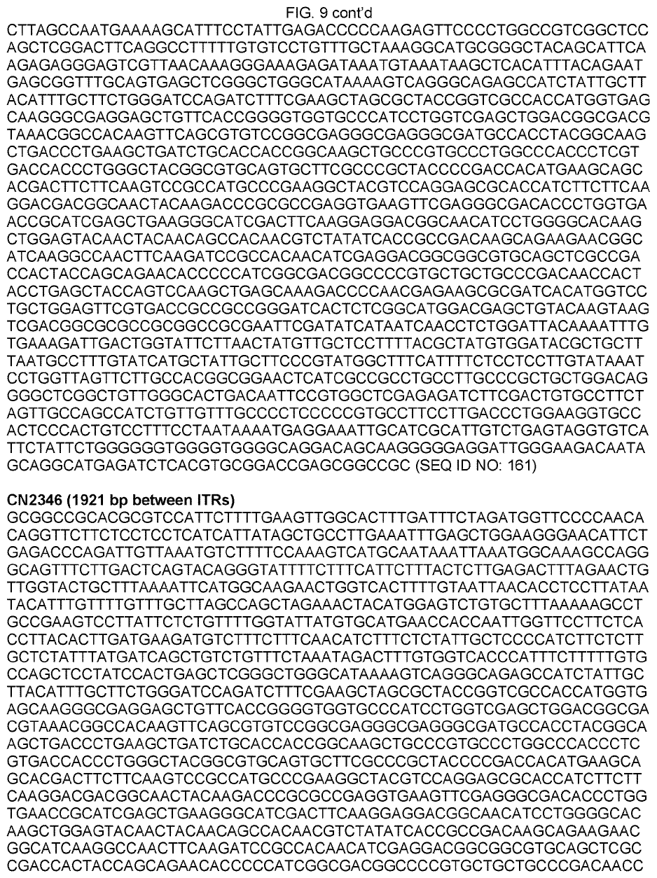

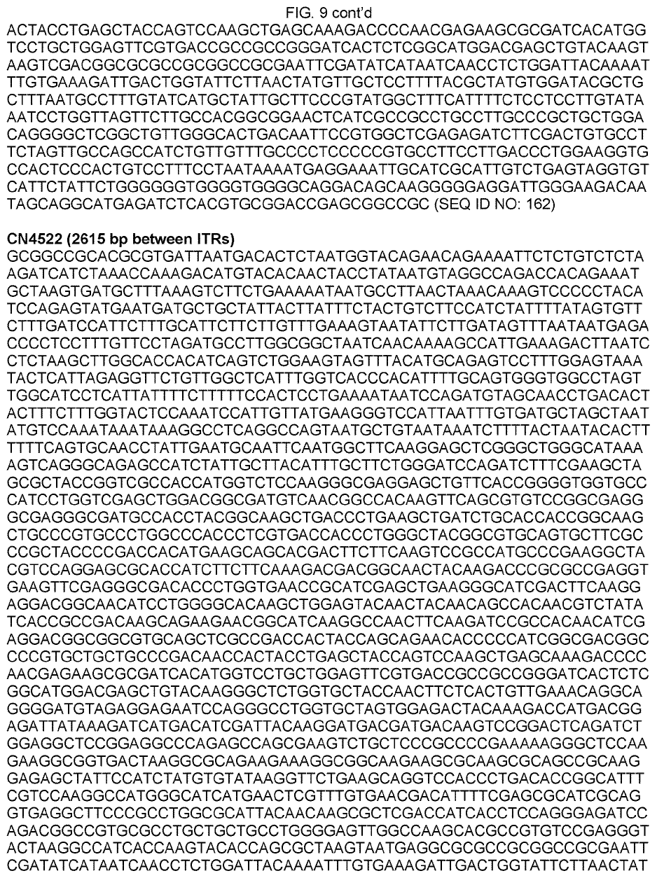

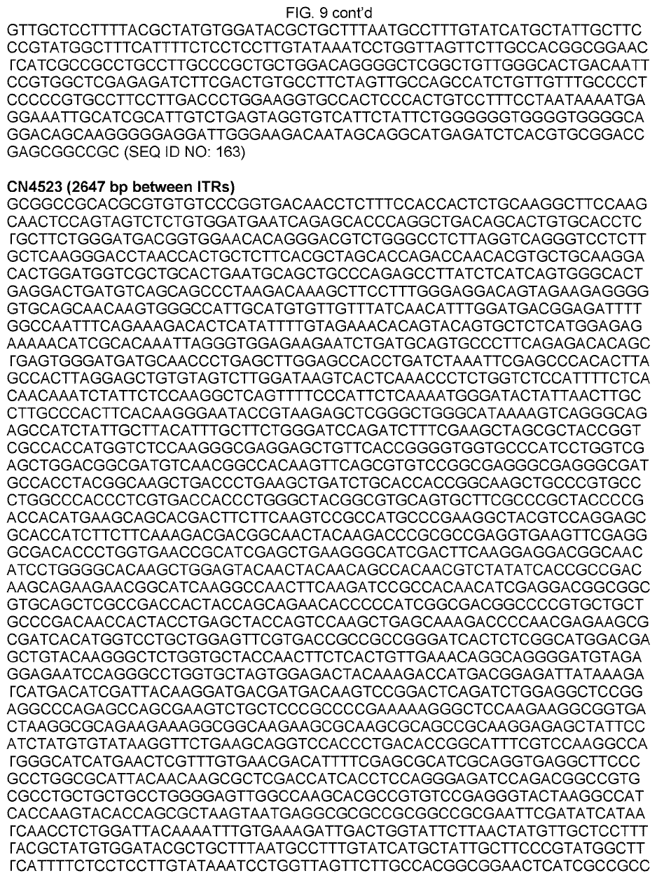

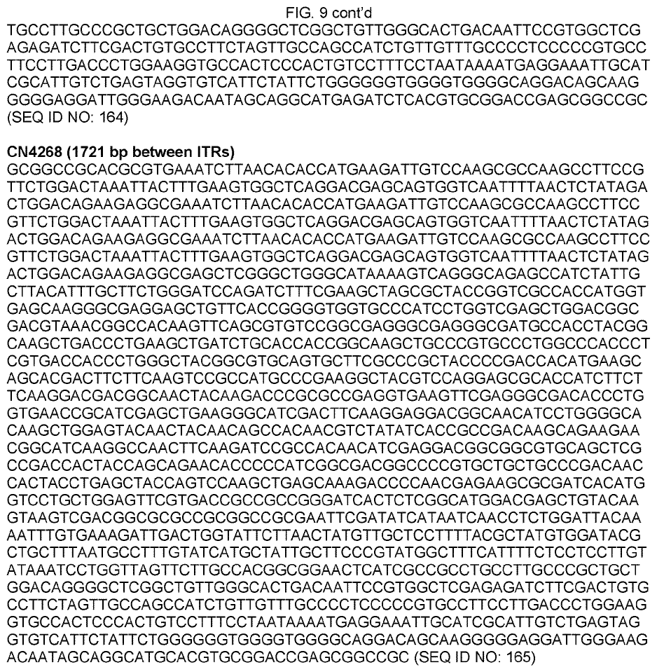

Subcomponent sequences within the larger vector sequences can be readily identified by one of ordinary skill in the art and based on the contents of the current disclosure (see FIG. 9). Nucleotides between identifiable and enumerated subcomponents reflect restriction enzyme recognition sites used in assembly (cloning) of the constructs, and in some cases, additional nucleotides do not convey any identifiable function. These segments of complete vector sequences can be adjusted based on use of different cloning strategies and/or vectors. In general, short 6-nucleotide palindromic sequences reflect vector construction artifacts that are not important to vector function.

In particular embodiments vectors (e.g., AAV) with capsids that cross the blood-brain barrier (BBB) are selected. In particular embodiments, vectors are modified to include capsids that cross the BBB. Examples of AAV with viral capsids that cross the blood brain barrier include AAV9 (Gombash et al., Front Mol Neurosci. 2014; 7:81), AAVrh. 10 (Yang, et al., Mol Ther. 2014; 22 (7): 1299-1309), AAV1R6, AAV1R7 (Albright et al., Mol Ther. 2018; 26 (2): 510), rAAVrh.8 (Yang, et al., supra), AAV-BR1 (Marchio et al., EMBO Mol Med. 2016; 8 (6): 592), AAV-PHP.S (Chan et al., Nat Neurosci. 2017; 20 (8): 1172), AAV-PHP.B (Deverman et al., Nat Biotechnol. 2016; 34 (2): 204), AAV-PPS (Chen et al., Nat Med. 2009; 15:1215), and PHP.eB. In particular embodiments, the PHP.eB capsid differs from AAV9 such that, using AAV9 as a reference, amino acids starting at residue 586: S-AQ-A (SEQ ID NO: 148) are changed to S-DGTLAVPFK-A (SEQ ID NO: 149). In particular embodiments, PHP.eb refers to SEQ ID NO: 83.

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31 (4): 317), for example, as described in relation to clinical trials for the treatment of spinal muscular atrophy (SMA) syndrome by AveXis (AVXS-101, NCT03505099) and the treatment of CLN3 gene-Related Neuronal Ceroid-Lipofuscinosis (NCT03770572).

AAVrh. 10, was originally isolated from rhesus macaques and shows low seropositivity in humans when compared with other common serotypes used for gene delivery applications (Selot et al., Front Pharmacol. 2017; 8:441) and has been evaluated in clinical trials LYS-SAF302, LYSOGENE, and NCT03612869.

AAV1R6 and AAV1R7, two variants isolated from a library of chimeric AAV vectors (AAV1 capsid domains swapped into AAVrh. 10), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 151) epitope that was isolated during in vivo screening of a random AAV display peptide library. It shows high specificity accompanied by high transgene expression in the brain with minimal off-target affinity (including for the liver) (Körbelin et al., EMBO Mol Med. 2016; 8 (6): 609).

AAV-PHP.S (Addgene, Watertown, MA) is a variant of AAV9 generated with the CREATE method that encodes the 7-mer sequence QAVRTSL (SEQ ID NO: 152), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 153). It transfers genes throughout the CNS with higher efficiency than AAV9 and transduces the majority of astrocytes and neurons across multiple CNS regions.

AAV-PPS, an AAV2 variant crated by insertion of the DSPAHPS (SEQ ID NO: 154) epitope into the capsid of AAV2, shows a dramatically improved brain tropism relative to AAV2.

For additional information regarding capsids that cross the blood brain barrier, see Chan et al., Nat. Neurosci. 2017 August: 20 (8): 1172-1179.

(ii) Compositions for Administration. Artificial expression constructs and vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in particular embodiments, when administered intravenously (e.g., at the retro-orbital plexus).

In particular embodiments, compositions can be formulated for intravenous, intraparenchymal, intraocular, intravitreal, parenteral, subcutaneous, intracerebro-ventricular, intramuscular, intrathecal, intraspinal, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24 (12): 1113-1128, 1998; Quintanar-Guerrero et al., Pharm Res. 15 (7): 1056-1062, 1998; Quintanar-Guerrero et al., J. Microencapsul. 15 (1): 107-119, 1998; Douglas et al., Crit Rev Ther Drug Carrier Syst 3 (3): 233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69 (2): 199-202, 1980; Couvreur et al., Crit Rev Ther Drug Carrier Syst. 5 (1) 1-20, 1988; zur Muhlen et al., Eur J Pharm Biopharm, 45 (2): 149-155, 1998; Zambaux et al., J Control Release 50 (1-3): 31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In particular embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al., Prog Retin Eye Res, 17 (1): 33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In particular embodiments, for administration to humans, compositions should meet sterility, pyrogenicity, and the general safety and purity standards as required by United States Food and Drug Administration (FDA) or other applicable regulatory agencies in other countries.

(iii) Cell Lines Including Artificial Expression Constructs. The present disclosure includes cells including an artificial expression construct described herein. A cell that has been transformed with an artificial expression construct can be used for many purposes, including in neuroanatomical studies, assessments of functioning and/or non-functioning proteins, and drug screens that assess the regulatory properties of enhancers.

A variety of host cell lines can be used, but in particular embodiments, the cell is a mammalian cell. In particular embodiments, the artificial express construct includes an enhancer and/or a vector sequence of eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, or eHGT_603m and/or CN3062, CN3063, CN2979, CN2498, CN2677, CN2505, CN2499, CN4004, CN3575, CN4254, CN4255, CN4256, CN2806, CN2807, CN2808, CN2809, CN2812, CN2813, CN2678, CN2679, CN2680, CN2681, CN2833, CN2834, CN2835, CN2836, CN2627, CN2675, CN2344, CN3060, CN3061, CN3098, CN2493, CN3568, CN2495, CN2496, CN2827, CN2846, CN3572, CN4253, CN4257, CN2410, CN2345, CN2346, CN4268, CN4522, or CN4523, and the cell line is a human, primate, or murine cell. Cell lines which can be utilized for transgenesis in the present disclosure also include primary cell lines derived from living tissue such as rat or mouse brains and organotypic cell cultures, including brain slices from animals such as rats, mice, non-human primates, or human neurosurgical tissue.

In particular embodiments, non-neuronal cell lines may be used, including mouse embryonic stem cells. Cultured mouse embryonic stem cells can be used to analyze expression of genetic constructs using transient transfection with plasmid constructs. Mouse embryonic stem cells are pluripotent and undifferentiated. These cells can be maintained in this undifferentiated state by Leukemia Inhibitory Factor (LIF). Withdrawal of LIF induces differentiation of the embryonic stem cells. In culture, the stem cells form a variety of differentiated cell types. Differentiation is caused by the expression of tissue specific transcription factors, allowing the function of an enhancer sequence to be evaluated. (See for example Fiskerstrand et al., FEBS Lett 458:171-174, 1999).

In particular embodiments, “neuronal” describes something that is of, related to, or includes, neuronal cells. Neuronal cells are defined by the presence of an axon and dendrites. The term “neuronal-specific” refers to something that is found, or an activity that occurs, in neuronal cells or cells derived from neuronal cells, but is not found in or occur in, or is not found substantially in or occur substantially in, non-neuronal cells or cells not derived from neuronal cells, for example glial cells such as astrocytes or oligodendrocytes.

U.S. Publication No. 2012/0329714 describes use of prolactin to increase neural stem cell numbers while U.S. Publication No. 2012/0308530 describes a culture surface with amino groups that promotes neuronal differentiation into neurons, astrocytes and oligodendrocytes. Thus, the fate of neural stem cells can be controlled by a variety of extracellular factors. Commonly used factors include brain derived growth factor (BDNF; Shetty and Turner, 1998, J. Neurobiol. 35:395-425); fibroblast growth factor (bFGF; U.S. Pat. No. 5,766,948; FGF-1, FGF-2); Neurotrophin-3 (NT-3) and Neurotrophin-4 (NT-4); Caldwell, et al., 2001, Nat. Biotechnol. 1; 19:475-9); ciliary neurotrophic factor (CNTF); BMP-2 (U.S. Pat. Nos. 5,948,428 and 6,001,654); isobutyl 3-methylxanthine; leukemia inhibitory growth factor (LIF; U.S. Pat. No. 6,103,530); somatostatin; amphiregulin; neurotrophins (e.g., cyclic adenosine monophosphate; epidermal growth factor (EGF); dexamethasone (glucocorticoid hormone); forskolin; GDNF family receptor ligands; potassium; retinoic acid (U.S. Pat. No. 6,395,546); tetanus toxin; and transforming growth factor-α and TGF-β (U.S. Pat. Nos. 5,851,832 and 5,753,506).

In particular embodiments, yeast one-hybrid systems may also be used to identify compounds that inhibit specific protein/DNA interactions, such as transcription factors for eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, or eHGT_603m.

Transgenic animals are described below. Cell lines may also be derived from such transgenic animals. For example, primary tissue culture from transgenic mice (e.g., also as described below) can provide cell lines with the artificial expression construct already integrated into the genome. (for an example see Mackenzie & Quinn, Proc Natl Acad Sci USA 96:15251-15255, 1999).

(iv) Transgenic Animals. Another aspect of the disclosure includes transgenic animals, the genome of which contains an artificial expression construct including eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, and/or eHGT_603m operatively linked to a heterologous coding sequence. In particular embodiments, the genome of a transgenic animal includes CN3062, CN3063, CN2979, CN2498, CN2677, CN2505, CN2499, CN4004, CN3575, CN4254, CN4255, CN4256, CN2806, CN2807, CN2808, CN2809, CN2812, CN2813, CN2678, CN2679, CN2680, CN2681, CN2833, CN2834, CN2835, CN2836, CN2627, CN2675, CN2344, CN3060, CN3061, CN3098, CN2493, CN3568, CN2495, CN2496, CN2827, CN2846, CN3572, CN4253, CN4257, CN2410, CN2345, CN2346, CN4268, CN4522, and/or CN4523. In particular embodiments, when a non-integrating vector is utilized, a transgenic animal includes an artificial expression construct including eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, and/or eHGT_603m, and/or CN3062, CN3063, CN2979, CN2498, CN2677, CN2505, CN2499, CN4004, CN3575, CN4254, CN4255, CN4256, CN2806, CN2807, CN2808, CN2809, CN2812, CN2813, CN2678, CN2679, CN2680, CN2681, CN2833, CN2834, CN2835, CN2836, CN2627, CN2675, CN2344, CN3060, CN3061, CN3098, CN2493, CN3568, CN2495, CN2496, CN2827, CN2846, CN3572, CN4253, CN4257, CN2410, CN2345, CN2346, CN4268, CN4522, and/or CN4523 within one or more of its cells.

Detailed methods for producing transgenic animals are described in U.S. Pat. No. 4,736,866. Transgenic animals may be of any nonhuman species, but preferably include nonhuman primates (NHPs), sheep, horses, cattle, pigs, goats, dogs, cats, rabbits, chickens, and rodents such as guinea pigs, hamsters, gerbils, rats, mice, and ferrets.

In particular embodiments, construction of a transgenic animal results in an organism that has an engineered construct present in all cells in the same genomic integration site. Thus, cell lines derived from such transgenic animals will be consistent in as much as the engineered construct will be in the same genomic integration site in all cells and hence will suffer the same position effect variegation. In contrast, introducing genes into cell lines or primary cell cultures can give rise to heterologous expression of the construct. A disadvantage of this approach is that the expression of the introduced DNA may be affected by the specific genetic background of the host animal.

As indicated above in relation to cell lines, the artificial expression constructs of this disclosure can be used to genetically modify mouse embryonic stem cells using techniques known in the art. Typically, the artificial expression construct is introduced into cultured murine embryonic stem cells. Transformed ES cells are then injected into a blastocyst from a host mother and the host embryo re-implanted into the mother. This results in a chimeric mouse whose tissues are composed of cells derived from both the embryonic stem cells present in the cultured cell line and the embryonic stem cells present in the host embryo. Usually, the mice from which the cultured ES cells used for transgenesis are derived are chosen to have a different coat color from the host mouse into whose embryos the transformed cells are to be injected. Chimeric mice will then have a variegated coat color. As long as the germ-line tissue is derived, at least in part, from the genetically modified cells, then the chimeric mice crossed with an appropriate strain can produce offspring that will carry the transgene.

In addition to the methods of delivery described above, the following techniques are also contemplated as alternative methods of delivering artificial expression constructs to target cells or targeted tissues and organs of an animal, and in particular, to cells, organs, or tissues of a vertebrate mammal: sonophoresis (e.g., ultrasound, as described in U.S. Pat. No. 5,656,016); intraosseous injection (U.S. Pat. No. 5,779,708); microchip devices (U.S. Pat. No. 5,797,898); ophthalmic formulations (Bourlais et al., Prog Retin Eye Res, 17 (1): 33-58, 1998); transdermal matrices (U.S. Pat. Nos. 5,770,219 and 5,783,208); feedback-controlled delivery (U.S. Pat. No. 5,697,899), and any other delivery method available and/or described elsewhere in the disclosure.

(v) Methods of Use. In particular embodiments, a composition including a physiologically active component described herein is administered to a subject to result in a physiological effect.

In particular embodiments, the disclosure includes the use of the artificial expression constructs described herein to modulate expression of a heterologous gene which is either partially or wholly encoded in a location downstream to that enhancer in an engineered sequence. Thus, there are provided herein methods of use of the disclosed artificial expression constructs in the research, study, and potential development of medicaments for preventing, treating or ameliorating the symptoms of a disease, dysfunction, or disorder.

Particular embodiments include methods of administering to a subject an artificial expression construct that includes eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, and/or eHGT_603m, and/or CN3062, CN3063, CN2979, CN2498, CN2677, CN2505, CN2499, CN4004, CN3575, CN4254, CN4255, CN4256, CN2806, CN2807, CN2808, CN2809, CN2812, CN2813, CN2678, CN2679, CN2680, CN2681, CN2833, CN2834, CN2835, CN2836, CN2627, CN2675, CN2344, CN3060, CN3061, CN3098, CN2493, CN3568, CN2495, CN2496, CN2827, CN2846, CN3572, CN4253, CN4257, CN2410, CN2345, CN2346, CN4268, CN4522, and/or CN4523 as described herein to drive expression of a gene in a targeted cell type. The subject can be an isolated cell, a network of cells, a tissue slice, an experimental animal, a veterinary animal, or a human.

As is well known in the medical arts, dosages for any one subject depends upon many factors, including the subject's size, surface area, age, the particular compound to be administered, sex, time and route of administration, general health, and other drugs being administered concurrently. Dosages for the compounds of the disclosure will vary, but, in particular embodiments, a dose could be from 105 to 10100 copies of an artificial expression construct of the disclosure. In particular embodiments, a patient receiving intravenous, intraparenchymal, intraspinal, retro-orbital, or intrathecal administration can be infused with from 106 to 1022 copies of the artificial expression construct.

An “effective amount” is the amount of a composition necessary to result in a desired physiological change in the subject. Effective amounts are often administered for research purposes. Effective amounts disclosed herein can cause a statistically-significant effect in an animal model, human study, in vivo, or in vitro assay.

The amount of expression constructs and time of administration of such compositions will be within the purview of the skilled artisan having benefit of the present teachings. It is likely, however, that the administration of effective amounts of the disclosed compositions may be achieved by a single administration, such as for example, a single injection of sufficient numbers of infectious particles to provide an effect in the subject. Alternatively, in some circumstances, it may be desirable to provide multiple, or successive administrations of the artificial expression construct compositions or other genetic constructs, either over a relatively short, or a relatively prolonged period of time, as may be determined by the individual overseeing the administration of such compositions. For example, the number of infectious particles administered to a mammal may be 107, 108, 109, 1010, 1011, 1012, 1013, or even higher, infectious particles/ml given either as a single dose or divided into two or more administrations as may be required to achieve an intended effect. In fact, in certain embodiments, it may be desirable to administer two or more different expression constructs in combination to achieve a desired effect.

In certain circumstances it will be desirable to deliver the artificial expression construct in suitably formulated compositions disclosed herein either by pipette, retro-orbital injection, subcutaneously, intraocularly, intravitreally, parenterally, subcutaneously, intravenously, intraparenchymally, intracerebro-ventricularly, intramuscularly, intrathecally, intraspinally, intraperitoneally, by oral or nasal inhalation, or by direct application or injection to one or more cells, tissues, or organs. The methods of administration may also include those modalities as described in U.S. Pat. Nos. 5,543,158; 5,641,515 and 5,399,363.

(vi) Kits and Commercial Packages. Kits and commercial packages contain an artificial expression construct described herein. The artificial expression construct can be isolated. In particular embodiments, the components of an expression product can be isolated from each other. In particular embodiments, the expression product can be within a vector, within a viral vector, within a cell, within a tissue slice or sample, and/or within a transgenic animal. Such kits may further include one or more reagents, restriction enzymes, peptides, therapeutics, pharmaceutical compounds, or means for delivery of the compositions such as syringes, injectables, and the like.

Embodiments of a kit or commercial package will also contain instructions regarding use of the included components, for example, in basic research, electrophysiological research, neuroanatomical research, and/or the research and/or treatment of a disorder, disease or condition.

The Exemplary Embodiments below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

(vii) Exemplary Embodiments.

1. An artificial enhancer comprising the core of an eHGT_374m, eHGT_375m, eHGT_380h, eHGT_387m, eHGT_390h, eHGT_400m, eHGT_401h, eHGT_409h, eHGT_410h, or eHGT_641m enhancer.

2. The artificial enhancer of embodiment 1, wherein the enhancer is human or murine.

3. The artificial enhancer of embodiments 1 or 2, wherein the core of the enhancer includes the sequence as set forth in SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 47, SEQ ID NO: 51, or SEQ ID NO: 53 or a sequence having at least 90% sequence identity to the sequence as set forth in SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 47, SEQ ID NO: 51, or SEQ ID NO: 53.

4. The artificial enhancer of any of embodiments 1-3, wherein the artificial enhancer includes 2, 3, 4, 5, 6, 7, 8, 9, or 10 copies of the enhancer core.

5. The artificial enhancer of embodiment 4, including 1, 2, 3, 4, 5, 6, 7, 8, 9, or 10 copies of the sequence as set forth in SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 47, SEQ ID NO: 51, or SEQ ID NO: 53 or a sequence having at least 90% sequence identity to the sequence as set forth in SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 47, SEQ ID NO: 51, or SEQ ID NO: 53.

6. The artificial enhancer of embodiments 4 or 5, including 1, 2, 3, 4, 5, 6, 7, 8, 9, or 10 copies of the sequence as set forth in SEQ ID NO: 8.

7. The artificial enhancer of embodiments 4 or 5 including 1, 2, 3, 4, 5, 6, 7, 8, 9, or 10 copies of the sequence as set forth in SEQ ID NO: 159.

8. The artificial enhancer of embodiments 4 or 5, including 1, 2, 3, 4, 5, 6, 7, 8, 9, or 10 copies of the sequence as set forth in SEQ ID NO: 10.

9. The artificial enhancer of embodiments 4 or 5, including 1, 2, 3, 4, 5, 6, 7, 8, 9, or 10 copies of the sequence as set forth in SEQ ID NO: 12.

10. The artificial enhancer of embodiments 4 or 5, including 1, 2, 3, 4, 5, 6, 7, 8, 9, or 10 copies of the sequence as set forth in SEQ ID NO: 14.

11. The artificial enhancer of embodiments 4 or 5, including 1, 2, 3, 4, 5, 6, 7, 8, 9, or 10 copies of the sequence as set forth in SEQ ID NO: 16.

12. The artificial enhancer of embodiments 4 or 5, including 1, 2, 3, 4, 5, 6, 7, 8, 9, or 10 copies of the sequence as set forth in SEQ ID NO: 39.

13. The artificial enhancer of embodiments 4 or 5, including 1, 2, 3, 4, 5, 6, 7, 8, 9, or 10 copies of the sequence as set forth in SEQ ID NO: 47.

14. The artificial enhancer of embodiments 4 or 5, including 1, 2, 3, 4, 5, 6, 7, 8, 9, or 10 copies of the sequence as set forth in SEQ ID NO: 51.

15. The artificial enhancer of embodiments 4 or 5, including 1, 2, 3, 4, 5, 6, 7, 8, 9, or 10 copies of the sequence as set forth in SEQ ID NO: 53.

16. The artificial enhancer of embodiment 6, including 3 copies of the sequence as set forth in SEQ ID NO: 8.

17. The artificial enhancer of embodiment 7, including 3 copies of the sequence as set forth in SEQ ID NO: 159.

18. The artificial enhancer of embodiment 8, including 3 copies of the sequence as set forth in SEQ ID NO: 10.

19. The artificial enhancer of embodiment 9, including 3 copies of the sequence as set forth in SEQ ID NO: 12.

20. The artificial enhancer of embodiment 10, including 3 copies of the sequence as set forth in SEQ ID NO: 14.

21. The artificial enhancer of embodiment 11, including 3 copies of the sequence as set forth in SEQ ID NO: 16.

22. The artificial enhancer of embodiment 12, including 3 copies of the sequence as set forth in SEQ ID NO: 39.

23. The artificial enhancer of embodiment 13, including 3 copies of the sequence as set forth in SEQ ID NO: 47.

24. The artificial enhancer of embodiment 14, including 3 copies of the sequence as set forth in SEQ ID NO: 51.

25. The artificial enhancer of embodiment 15, including 3 copies of the sequence as set forth in SEQ ID NO: 53.

26. The artificial enhancer of embodiment 16, wherein the artificial enhancer includes a sequence as set forth in SEQ ID NO: 9 or a sequence having at least 90% sequence identity to the sequence as set forth in SEQ ID NO: 9.

27. The artificial enhancer of embodiment 17, wherein the artificial enhancer includes a sequence as set forth in SEQ ID NO: 160 or a sequence having at least 90% sequence identity to the sequence as set forth in SEQ ID NO: 160.

28. The artificial enhancer of embodiment 18, wherein the artificial enhancer includes a sequence as set forth in SEQ ID NO: 11 or a sequence having at least 90% sequence identity to the sequence as set forth in SEQ ID NO: 11.

29. The artificial enhancer of embodiment 19, wherein the artificial enhancer includes a sequence as set forth in SEQ ID NO: 13 or a sequence having at least 90% sequence identity to the sequence as set forth in SEQ ID NO: 13.

30. The artificial enhancer of embodiment 20, wherein the artificial enhancer includes a sequence as set forth in SEQ ID NO: 15 or a sequence having at least 90% sequence identity to the sequence as set forth in SEQ ID NO: 15.

31. The artificial enhancer of embodiment 21, wherein the artificial enhancer includes a sequence as set forth in SEQ ID NO: 17 or a sequence having at least 90% sequence identity to the sequence as set forth in SEQ ID NO: 17.

32. The artificial enhancer of embodiment 22, wherein the artificial enhancer includes a sequence as set forth in SEQ ID NO: 40 or a sequence having at least 90% sequence identity to the sequence as set forth in SEQ ID NO: 40.

33. The artificial enhancer of embodiment 23, wherein the artificial enhancer includes a sequence as set forth in SEQ ID NO: 48 or a sequence having at least 90% sequence identity to the sequence as set forth in SEQ ID NO: 48.

34. The artificial enhancer of embodiment 24, wherein the artificial enhancer includes a sequence as set forth in SEQ ID NO: 52 or a sequence having at least 90% sequence identity to the sequence as set forth in SEQ ID NO: 52.

35. The artificial enhancer of embodiment 25, wherein the artificial enhancer includes a sequence as set forth in SEQ ID NO: 54 or a sequence having at least 90% sequence identity to the sequence as set forth in SEQ ID NO: 54.

36. An artificial expression construct including (i) an enhancer selected from eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, and eHGT_603m; (ii) a promoter; and (iii) a heterologous coding sequence.

37. The artificial expression construct of embodiment 36, wherein the heterologous coding sequence encodes an effector element or an expressible element.

38. The artificial expression construct of embodiment 37, wherein the effector element includes a reporter protein or a functional molecule.

39. The artificial expression construct of embodiment 38, wherein the reporter protein includes a fluorescent protein.

40. The artificial expression construct of embodiment 38, wherein the functional molecule includes a functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or a designer receptor exclusively activated by designer drug (DREADD).

41. The artificial expression construct of embodiment 37, wherein the expressible element includes a non-functional molecule.

42. The artificial expression construct of embodiment 41, wherein the non-functional molecule includes a non-functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

43. The artificial expression construct of any of embodiments 36-42, wherein the artificial expression construct is associated with a capsid that crosses the blood brain barrier.

44. The artificial expression construct of embodiment 43, wherein the capsid includes PHP.eB, AAV9, AAVrh. 10, AAV-BR1, AAV-PHP.S, AAV-PHP.B, or AAV-PPS.

45. The artificial expression construct of any of embodiments 36-44, wherein the artificial expression construct includes or encodes a skipping element.

46. The artificial expression construct of embodiment 45, wherein the skipping element includes a 2A peptide and/or an internal ribosome entry site (IRES).

47. The artificial expression construct of embodiment 46, wherein the 2A peptide includes T2A, P2A, E2A, or F2A.

48. The artificial expression construct of any of embodiments 36-47, wherein the artificial expression construct includes or encodes a set of features selected from: eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, eHGT_603m, AAV, scAAV, rAAV, pAAV, minBglobin, CMV, minCMV, minCMV*, minRho, minRho*, fluorescent protein (e.g., EGFP, SYFP, GFP), hsA2, Cre, iCre, dgCre, FlpO, tTA2, SP10 (e.g., 3xSP10), tag cassette, 10aa, nuclear localization protein, self-cleaving peptides, WPRE, WPRE3, hGHpA, and/or BGHpA.

49. The artificial expression construct of any of embodiments 36-48, wherein the artificial expression construct includes or encodes a set of features selected from:
eHGT_409 h-minBglobin-[heterologous coding sequence]-[post-regulatory elements];
eHGT_410 h-minBglobin-[heterologous coding sequence]-[post-regulatory elements];
eHGT_361 h-minBglobin-[heterologous coding sequence]-[post-regulatory elements];
eHGT_395m-minBglobin-[heterologous coding sequence]-[post-regulatory elements];
eHGT_401 h-minBglobin-[heterologous coding sequence]-[post-regulatory elements];
eHGT_391 h-minBglobin-[heterologous coding sequence]-[post-regulatory elements];
eHGT_403 h-minBglobin-[heterologous coding sequence]-[post-regulatory elements];
3xCore_eHGT_641m-minBglobin-[heterologous coding sequence]-[post-regulatory elements];
3xcore2_eHGT_400m-minBglobin-[heterologous coding sequence]-[post-regulatory elements];
3xcore2_eHGT_401 h-minBglobin-[heterologous coding sequence]-[post-regulatory elements];
3xcore2_eHGT_409 h-minBglobin-[heterologous coding sequence]-[post-regulatory elements];
3xcore2_eHGT_410 h-minBglobin-[heterologous coding sequence]-[post-regulatory elements];
eHGT_363 h-minBglobin-[heterologous coding sequence]-[post-regulatory elements];
eHGT_363 h-minBglobin-[heterologous coding sequence]-[skipping element]-[tag cassette]-10aa-[nuclear localization protein]-[post-regulatory elements];
eHGT_331 h-minBglobin-[heterologous coding sequence]-[post-regulatory elements];
eHGT_332 h-minBglobin-[heterologous coding sequence]-[post-regulatory elements];
eHGT_333 h-minBglobin-[heterologous coding sequence]-[post-regulatory elements];
eHGT_335 h-minBglobin-[heterologous coding sequence]-[post-regulatory elements];
eHGT_334 h-minBglobin-[heterologous coding sequence]-[post-regulatory elements];
eHGT_360 h-minBglobin-[heterologous coding sequence]-[post-regulatory elements];
eHGT_562 h-minBglobin-[heterologous coding sequence]-[post-regulatory elements];
eHGT_563 h-minBglobin-[heterologous coding sequence]-[post-regulatory elements];
eHGT_564 h-minBglobin-[heterologous coding sequence]-[post-regulatory elements];
eHGT_565 h-minBglobin-[heterologous coding sequence]-[post-regulatory elements];
eHGT_566m-minBglobin-[heterologous coding sequence]-[post-regulatory elements];
eHGT_567m-minBglobin-[heterologous coding sequence]-[post-regulatory elements];
eHGT_568m-minBglobin-[heterologous coding sequence]-[post-regulatory elements];
eHGT_569m-minBglobin-[heterologous coding sequence]-[post-regulatory elements];
eHGT_734m-minBglobin-[heterologous coding sequence]-[post-regulatory elements];
eHGT_312 h-minBglobin-[heterologous coding sequence]-[post-regulatory elements];
eHGT_313 h-minBglobin-[heterologous coding sequence]-[post-regulatory elements];
eHGT_315 h-minBglobin-[heterologous coding sequence]-[post-regulatory elements];
eHGT_316 h-minBglobin-[heterologous coding sequence]-[post-regulatory elements];
eHGT_377m-minBglobin-[heterologous coding sequence]-[post-regulatory elements];
eHGT_389m-minBglobin-[heterologous coding sequence]-[post-regulatory elements];
eHGT_380 h-minBglobin-[heterologous coding sequence]-[post-regulatory elements];
eHGT_374 h-minBglobin-[heterologous coding sequence]-[post-regulatory elements];
3xcore1_eHGT_387m-minBglobin-[heterologous coding sequence]-[post-regulatory elements];
eHGT_385 h-minBglobin-[heterologous coding sequence]-[post-regulatory elements];
eHGT_386 h-minBglobin-[heterologous coding sequence]-[post-regulatory elements];
eHGT_378m-minBglobin-[heterologous coding sequence]-[post-regulatory elements];
eHGT_642m-minBglobin-[heterologous coding sequence]-[post-regulatory elements];
3xcore2_eHGT_390 h-minBglobin-[heterologous coding sequence]-[post-regulatory elements];
3xcore2_eHGT_380 h-minBglobin-[heterologous coding sequence]-[post-regulatory elements];
3xcore2_eHGT_374m-minBglobin-[heterologous coding sequence]-[post-regulatory elements];
3xcore2_eHGT_375m-minBglobin-[heterologous coding sequence]-[post-regulatory elements];
eHGT_358 h-minBglobin-[heterologous coding sequence]-[post-regulatory elements];
eHGT_358 h-minBglobin-[heterologous coding sequence]-[skipping element]-[tag cassette]-10aa-[nuclear localization protein]-[post-regulatory elements];
eHGT_603m-minBglobin-[heterologous coding sequence]-[post-regulatory elements];
eHGT_409h-[minimal promoter]-[heterologous coding sequence]-[post-regulatory elements];
eHGT_410h-[minimal promoter]-[heterologous coding sequence]-[post-regulatory elements];
eHGT_361h-[minimal promoter]-[heterologous coding sequence]-[post-regulatory elements];
eHGT_395m-[minimal promoter]-[heterologous coding sequence]-[post-regulatory elements];
eHGT_401h-[minimal promoter]-[heterologous coding sequence]-[post-regulatory elements];
eHGT_391h-[minimal promoter]-[heterologous coding sequence]-[post-regulatory elements];
eHGT_403h-[minimal promoter]-[heterologous coding sequence]-[post-regulatory elements];
3xCore_eHGT_641m-[minimal promoter]-[heterologous coding sequence]-[post-regulatory elements];
3xcore2_eHGT_400m-[minimal promoter]-[heterologous coding sequence]-[post-regulatory elements];
3xcore2_eHGT_401h-[minimal promoter]-[heterologous coding sequence]-[post-regulatory elements];
3xcore2_eHGT_409h-[minimal promoter]-[heterologous coding sequence]-[post-regulatory elements];
3xcore2_eHGT_410h-[minimal promoter]-[heterologous coding sequence]-[post-regulatory elements];
eHGT_363h-[minimal promoter]-[heterologous coding sequence]-[post-regulatory elements];
eHGT_363h-[minimal promoter]-[heterologous coding sequence]-[skipping element]-[tag cassette]-10aa-[nuclear localization protein]-[post-regulatory elements];
eHGT_331h-[minimal promoter]-[heterologous coding sequence]-[post-regulatory elements];
eHGT_332h-[minimal promoter]-[heterologous coding sequence]-[post-regulatory elements];
eHGT_333h-[minimal promoter]-[heterologous coding sequence]-[post-regulatory elements];
eHGT_335h-[minimal promoter]-[heterologous coding sequence]-[post-regulatory elements];
eHGT_334h-[minimal promoter]-[heterologous coding sequence]-[post-regulatory elements];
eHGT_360h-[minimal promoter]-[heterologous coding sequence]-[post-regulatory elements];
eHGT_562h-[minimal promoter]-[heterologous coding sequence]-[post-regulatory elements];
eHGT_563h-[minimal promoter]-[heterologous coding sequence]-[post-regulatory elements];
eHGT_564h-[minimal promoter]-[heterologous coding sequence]-[post-regulatory elements];
eHGT_565h-[minimal promoter]-[heterologous coding sequence]-[post-regulatory elements];
eHGT_566m-[minimal promoter]-[heterologous coding sequence]-[post-regulatory elements];
eHGT_567m-[minimal promoter]-[heterologous coding sequence]-[post-regulatory elements];
eHGT_568m-[minimal promoter]-[heterologous coding sequence]-[post-regulatory elements];
eHGT_569m-[minimal promoter]-[heterologous coding sequence]-[post-regulatory elements];
eHGT_734m-[minimal promoter]-[heterologous coding sequence]-[post-regulatory elements];
eHGT_312h-[minimal promoter]-[heterologous coding sequence]-[post-regulatory elements];
eHGT_313h-[minimal promoter]-[heterologous coding sequence]-[post-regulatory elements];
eHGT_315h-[minimal promoter]-[heterologous coding sequence]-[post-regulatory elements];
eHGT_316h-[minimal promoter]-[heterologous coding sequence]-[post-regulatory elements];
eHGT_377m-[minimal promoter]-[heterologous coding sequence]-[post-regulatory elements];
eHGT_389m-[minimal promoter]-[heterologous coding sequence]-[post-regulatory elements];
eHGT_380h-[minimal promoter]-[heterologous coding sequence]-[post-regulatory elements];
eHGT_374h-[minimal promoter]-[heterologous coding sequence]-[post-regulatory elements];
3xcore1_eHGT_387m-[minimal promoter]-[heterologous coding sequence]-[post-regulatory elements];
eHGT_385h-[minimal promoter]-[heterologous coding sequence]-[post-regulatory elements];
eHGT_386h-[minimal promoter]-[heterologous coding sequence]-[post-regulatory elements];
eHGT_378m-[minimal promoter]-[heterologous coding sequence]-[post-regulatory elements];
eHGT_642m-[minimal promoter]-[heterologous coding sequence]-[post-regulatory elements];
3xcore2_eHGT_390h-[minimal promoter]-[heterologous coding sequence]-[post-regulatory elements];
3xcore2_eHGT_380h-[minimal promoter]-[heterologous coding sequence]-[post-regulatory elements];
3xcore2_eHGT_374m-[minimal promoter]-[heterologous coding sequence]-[post-regulatory elements];
3xcore2_eHGT_375m-[minimal promoter]-[heterologous coding sequence]-[post-regulatory elements];
eHGT_358h-[minimal promoter]-[heterologous coding sequence]-[post-regulatory elements];
eHGT_358h-[minimal promoter]-[heterologous coding sequence]-[skipping element]-[tag cassette]-10aa-[nuclear localization protein]-[post-regulatory elements]; or
eHGT_603m-[minimal promoter]-[heterologous coding sequence]-[post-regulatory elements].

50. A vector including an artificial expression construct of any of embodiments 36-49.

51. The vector of embodiment 50, wherein the vector includes a viral vector.

52. The vector of embodiments 50 or 51, wherein the viral vector includes a recombinant adeno-associated viral (AAV) vector.

53. An adeno-associated viral (AAV) vector including at least one heterologous coding sequence, wherein the heterologous coding sequence is under the transcriptional control of a promoter and an enhancer selected eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, eHGT_363h, eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, eHGT_734m, eHGT_312h, eHGT_313h, eHGT_315h, eHGT_316h, eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, eHGT_358h, and eHGT_603m.

54. The AAV vector of embodiment 53, wherein the heterologous coding sequence encodes an effector element or an expressible element.

55. The AAV vector of embodiment 54, wherein the effector element includes a reporter protein or a functional molecule.

56. The AAV vector of embodiment 55, wherein the reporter protein includes a fluorescent protein.

57. The AAV vector of embodiment 55, wherein the functional molecule includes a functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

58. The AAV vector of embodiment 54, wherein the expressible element includes a non-functional molecule.

59. The AAV vector of embodiment 58, wherein the non-functional molecule includes a non-functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

60. A transgenic cell including an artificial expression construct or a vector of any of the preceding embodiments.

61. The transgenic cell of embodiment 60, wherein the transgenic cell is a non-neuronal cell.

62. The transgenic cell of embodiments 60 or 61, wherein the transgenic cell includes a glial cell.

63. The transgenic cell of embodiment 62, wherein the glial cell is an oligodendrocyte, microglia, or astrocyte.

64. The transgenic cell of embodiment 63, wherein the astrocyte is an L1 interlaminar astrocyte.

65. The transgenic cell of embodiments 60 or 61, wherein the transgenic cell is an endothelial cell.

66. The transgenic cell of any of embodiments 60-65, wherein the transgenic cell is murine, human, or non-human primate.

67. A non-human transgenic animal including an artificial expression construct, a vector, and/or a transgenic cell of any of the preceding embodiments.

68. The non-human transgenic animal of embodiment 67, wherein the non-human transgenic animal is a mouse or a non-human primate.

69. An administrable composition including an artificial expression construct, a vector, and/or a transgenic cell of any of the preceding embodiments.

70. A kit including an artificial expression construct, a vector, a transgenic cell, a non-human transgenic animal, and/or administrable compositions of any of the preceding embodiments.

71. A method for expressing a gene within a targeted population of non-neuronal cells in vivo or in vitro, the method including providing the administrable composition of embodiment 69 in a sufficient dosage and for a sufficient time to a sample or subject including the targeted population of non-neuronal cells and other cells thereby expressing the gene within the targeted population of non-neuronal cells.

72. The method of embodiment 71, wherein the gene encodes an effector element or an expressible element.

73. The method of embodiment 72, wherein the effector element includes a reporter protein or a functional molecule.

74. The method of embodiment 73, wherein the reporter protein includes a fluorescent protein.

75. The method of embodiment 73, wherein the functional molecule includes a functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

76. The method of embodiment 72, wherein the expressible element includes a non-functional molecule.

77. The method of embodiment 76, wherein the non-functional molecule includes a non-functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

78. The method of any of embodiments 71-77, wherein the providing includes pipetting.

79. The method of embodiment 78, wherein the pipetting is to a brain slice.

80. The method of embodiment 79, wherein the brain slice includes neuronal cells and the targeted population of non-neuronal cells.

81. The method of embodiment 79, wherein the brain slice includes a glial cell.

82. The method of embodiment 81, wherein the glial cell includes an oligodendrocyte, microglia, or astrocyte.

1. The method of claim 82, wherein the astrocyte comprises an L1 interlaminar astrocyte.

83. The method of embodiment 79, wherein the brain slice includes an endothelial cell.

84. The method of any of embodiments 79-83, wherein the brain slice is murine, human, or non-human primate.

85. The method of any of embodiments 878-84, wherein the providing includes administering to a living subject.

86. The method of embodiment 85, wherein the living subject is a human, non-human primate, or a mouse.

87. The method of embodiments 85 or 86, wherein the administering to a living subject is through injection.

88. The method of embodiment 87, wherein the injection includes intravenous injection, intraparenchymal injection into brain tissue, intracerebroventricular (ICV) injection, intra-cisterna magna (ICM) injection, or intrathecal injection.

89. An artificial expression construct including a sequence as set forth in SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 131, SEQ ID NO: 133, SEQ ID NO: 134, SEQ ID NO: 135, SEQ ID NO: 161, SEQ ID NO: 162, SEQ ID NO: 163, SEQ ID NO: 164, or SEQ ID NO: 165 or a sequence having at least 90% sequence identity to a sequence as set forth in SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 131, SEQ ID NO: 133, SEQ ID NO: 134, SEQ ID NO: 135, SEQ ID NO: 161, SEQ ID NO: 162, SEQ ID NO: 163, SEQ ID NO: 164, or SEQ ID NO: 165.

(viii) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (11e), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, 11e, Phe, Met, and Trp; Group 11 (aliphatic): Gly, Ala, Val, Leu, and 11e; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: 11e (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); 11e (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The term concatenate is broadly used to describe linking together into a chain or series. It is used to describe the linking together of nucleotide or amino acid sequences into a single nucleotide or amino acid sequence, respectively. The term “concatamerize” should be interpreted to recite: “concatenate.”

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in targeted expression in the targeted cell population as determined by scRNA-Seq and the following (targeted cell population/enhancer(s)):
oligodendrocytes/eHGT_409h, eHGT_410h, eHGT_361h, eHGT_395m, eHGT_401h, eHGT_391h, eHGT_403h, 3xCore_eHGT_641m, 3xcore2_eHGT_400m, 3xcore2_eHGT_401h, 3xcore2_eHGT_409h, 3xcore2_eHGT 410h, and eHGT_363h;
microglia/eHGT_331h, eHGT_332h, eHGT_333h, eHGT_335h, eHGT_334h, eHGT_360h, eHGT_562h, eHGT_563h, eHGT_564h, eHGT_565h, eHGT_566m, eHGT_567m, eHGT_568m, eHGT_569m, and eHGT_734m;
astrocytes/eHGT_377m, eHGT_389m, eHGT_380h, eHGT_374h, 3xcore1_eHGT_387m, eHGT_385h, eHGT_386h, eHGT_378m, eHGT_642m, 3xcore2_eHGT_390h, 3xcore2_eHGT_380h, 3xcore2_eHGT_374m, 3xcore2_eHGT_375m, and eHGT_358h;
L1 interlaminar astrocytes/eHGT_312h, eHGT_313h, eHGT_315h, and eHGT_316h; and
endothelial cells/eHGT_603m.

In particular embodiments, artificial means not naturally occurring.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An artificial expression construct comprising (i) a concatenated core of an enhancer, wherein the core of the enhancer comprises the sequence as set forth in SEQ ID NO: 47; (ii) a promoter; and (iii) a heterologous coding sequence.

2. An artificial enhancer comprising an enhancer core, wherein the enhancer core comprises the sequence as set forth in SEQ ID NO: 47, SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 51, or SEQ ID NO: 53 or has a sequence having at least 90% sequence identity to the sequence as set forth in SEQ ID NO: 47, SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 51, or SEQ ID NO: 53.

3. The artificial enhancer of claim 2, wherein the enhancer is human or murine.

4. The artificial enhancer of claim 2, wherein the artificial enhancer has 1, 2, 3, 4, 5, 6, 7, 8, 9, or 10 copies of the enhancer core.

5. The artificial enhancer of claim 4, having 1, 2, 3, 4, 5, 6, 7, 8, 9, or 10 copies of the sequence as set forth in SEQ ID NO: 47, SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 51, or SEQ ID NO: 53 or a sequence having at least 90% sequence identity to the sequence as set forth in SEQ ID NO: 47, SEQ ID NO: 8, SEQ ID NO: 159, SEQ ID NO: 10, SEQ ID NO: 12, SEQ ID NO: 14, SEQ ID NO: 16, SEQ ID NO: 39, SEQ ID NO: 51, or SEQ ID NO: 53.

6. The artificial enhancer of claim 5, having 1, 2, 3, 4, 5, 6, 7, 8, 9, or 10 copies of the sequence as set forth in SEQ ID NO: 47.

7. The artificial enhancer of claim 5, having 1, 2, 3, 4, 5, 6, 7, 8, 9, or 10 copies of the sequence as set forth in SEQ ID NO: 8.

8. The artificial enhancer of claim 5, having 2, 3, 4, 5, 6, 7, 8, 9, or 10 copies of the sequence as set forth in SEQ ID NO: 159.

9. The artificial enhancer of claim 5, having 1, 2, 3, 4, 5, 6, 7, 8, 9, or 10 copies of the sequence as set forth in SEQ ID NO: 10.

10. The artificial enhancer of claim 5, having 1, 2, 3, 4, 5, 6, 7, 8, 9, or 10 copies of the sequence as set forth in SEQ ID NO: 12.

11. The artificial enhancer of claim 5, having 1, 2, 3, 4, 5, 6, 7, 8, 9, or 10 copies of the sequence as set forth in SEQ ID NO: 14.

12. The artificial enhancer of claim 5, having 1, 2, 3, 4, 5, 6, 7, 8, 9, or 10 copies of the sequence as set forth in SEQ ID NO: 16.

13. The artificial enhancer of claim 5, having 1, 2, 3, 4, 5, 6, 7, 8, 9, or 10 copies of the sequence as set forth in SEQ ID NO: 39.

14. The artificial enhancer of claim 5, having 1, 2, 3, 4, 5, 6, 7, 8, 9, or 10 copies of the sequence as set forth in SEQ ID NO: 51.

15. The artificial enhancer of claim 5, having 1, 2, 3, 4, 5, 6, 7, 8, 9, or 10 copies of the sequence as set forth in SEQ ID NO: 53.

16. The artificial enhancer of claim 6, having 3 copies of the sequence as set forth in SEQ ID NO: 47.

17. The artificial enhancer of claim 7, having 3 copies of the sequence as set forth in SEQ ID NO: 8.

18. The artificial enhancer of claim 8, having 3 copies of the sequence as set forth in SEQ ID NO: 159.

19. The artificial enhancer of claim 9, having 3 copies of the sequence as set forth in SEQ ID NO: 10.

20. The artificial enhancer of claim 10, having 3 copies of the sequence as set forth in SEQ ID NO: 12.

21. The artificial enhancer of claim 11, having 3 copies of the sequence as set forth in SEQ ID NO: 14.

22. The artificial enhancer of claim 12, having 3 copies of the sequence as set forth in SEQ ID NO: 16.

23. The artificial enhancer of claim 13, having 3 copies of the sequence as set forth in SEQ ID NO: 39.

24. The artificial enhancer of claim 14, having 3 copies of the sequence as set forth in SEQ ID NO: 51.

25. The artificial enhancer of claim 15, having 3 copies of the sequence as set forth in SEQ ID NO: 53.

26. The artificial enhancer of claim 16, wherein the artificial enhancer has a sequence as set forth in SEQ ID NO: 48 or a sequence having at least 90% sequence identity to the sequence as set forth in SEQ ID NO: 48.

27. The artificial enhancer of claim 17, wherein the artificial enhancer has a sequence as set forth in SEQ ID NO: 9 or a sequence having at least 90% sequence identity to the sequence as set forth in SEQ ID NO: 9.

28. The artificial enhancer of claim 18, wherein the artificial enhancer has a sequence as set forth in SEQ ID NO: 160 or a sequence having at least 90% sequence identity to the sequence as set forth in SEQ ID NO: 160.

29. The artificial enhancer of claim 19, wherein the artificial enhancer has a sequence as set forth in SEQ ID NO: 11 or a sequence having at least 90% sequence identity to the sequence as set forth in SEQ ID NO: 11.

30. The artificial enhancer of claim 20, wherein the artificial enhancer has a sequence as set forth in SEQ ID NO: 13 or a sequence having at least 90% sequence identity to the sequence as set forth in SEQ ID NO: 13.

31. The artificial enhancer of claim 21, wherein the artificial enhancer has a sequence as set forth in SEQ ID NO: 15 or a sequence having at least 90% sequence identity to the sequence as set forth in SEQ ID NO: 15.

32. The artificial enhancer of claim 22, wherein the artificial enhancer has a sequence as set forth in SEQ ID NO: 17 or a sequence having at least 90% sequence identity to the sequence as set forth in SEQ ID NO: 17.

33. The artificial enhancer of claim 23, wherein the artificial enhancer has a sequence as set forth in SEQ ID NO: 40 or a sequence having at least 90% sequence identity to the sequence as set forth in SEQ ID NO: 40.

34. The artificial enhancer of claim 24, wherein the artificial enhancer has a sequence as set forth in SEQ ID NO: 52 or a sequence having at least 90% sequence identity to the sequence as set forth in SEQ ID NO: 52.

35. The artificial enhancer of claim 25, wherein the artificial enhancer has a sequence as set forth in SEQ ID NO: 54 or a sequence having at least 90% sequence identity to the sequence as set forth in SEQ ID NO: 54.

36. An artificial expression construct comprising (i) an enhancer selected from SEQ ID NO: 48, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 158, SEQ ID NO: SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 40, SEQ ID NO: 41, SEQ ID NO: 42, SEQ ID NO: 43, SEQ ID NO: 44, SEQ ID NO: 52, SEQ ID NO:54, SEQ ID NO: 160, and SEQ ID NO: 157; (ii) a promoter; and (iii) a heterologous coding sequence.

37. The artificial expression construct of claim 36, wherein the heterologous coding sequence encodes an effector element or an expressible element.

38. The artificial expression construct of claim 37, wherein the effector element comprises a reporter protein or a functional molecule.

39. The artificial expression construct of claim 38, wherein the reporter protein comprises a fluorescent protein.

40. The artificial expression construct of claim 38, wherein the functional molecule comprises a functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or a designer receptor exclusively activated by designer drug (DREADD).

41. The artificial expression construct of claim 37, wherein the expressible element comprises a non-functional molecule.

42. The artificial expression construct of claim 41, wherein the non-functional molecule comprises a non-functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

43. The artificial expression construct of claim 36, wherein the artificial expression construct is associated with a capsid that crosses the blood brain barrier.

44. The artificial expression construct of claim 43, wherein the capsid comprises PHP.eB, AAV9, AAVrh.10, AAV-BR1, AAV-PHP.S, AAV-PHP.B, or AAV-PPS.

45. The artificial expression construct of claim 36, wherein the artificial expression construct comprises or encodes a skipping element.

46. The artificial expression construct of claim 45, wherein the skipping element comprises a 2A peptide or an internal ribosome entry site (IRES).

47. The artificial expression construct of claim 46, wherein the 2A peptide comprises T2A, P2A, E2A, or F2A.

48. The artificial expression construct of claim 36, wherein the artificial expression construct comprises or encodes a set of features selected from: SEQ ID NO: 48, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 158, SEQ ID NO: SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 40, SEQ ID NO: 41, SEQ ID NO: 42, SEQ ID NO: 43, SEQ ID NO: 44, SEQ ID NO: 52, SEQ ID NO:54, SEQ ID NO: 160, SEQ ID NO: 157, AAV, scAAV, rAAV, pAAV, minBglobin, CMV, minCMV, minCMV*, minRho, minRho*, fluorescent protein, hsA2, Cre, iCre, dgCre, FlpO, tTA2, SP10, tag cassette, 10aa, nuclear localization protein, self-cleaving peptides, WPRE, WPRE3, hGHpA, and/or BGHpA.

49. The artificial expression construct of claim 36, wherein the artificial expression construct comprises or encodes a set of features selected from:
3xcore2_eHGT_390 h-minBglobin-[heterologous coding sequence]-[post-regulatory elements];
eHGT_409 h-minBglobin-[heterologous coding sequence]-[post-regulatory elements];
eHGT_410 h-minBglobin-[heterologous coding sequence]-[post-regulatory elements];
eHGT_361 h-minBglobin-[heterologous coding sequence]-[post-regulatory elements];
eHGT_395m-minBglobin-[heterologous coding sequence]-[post-regulatory elements];
eHGT_401 h-minBglobin-[heterologous coding sequence]-[post-regulatory elements];
eHGT_391 h-minBglobin-[heterologous coding sequence]-[post-regulatory elements];
eHGT_403 h-minBglobin-[heterologous coding sequence]-[post-regulatory elements];
3xCore_eHGT_641m-minBglobin-[heterologous coding sequence]-[post-regulatory elements];
3xcore2_eHGT_400m-minBglobin-[heterologous coding sequence]-[post-regulatory elements];
3xcore2_eHGT_401 h-minBglobin-[heterologous coding sequence]-[post-regulatory elements];
3xcore2_eHGT_409 h-minBglobin-[heterologous coding sequence]-[post-regulatory elements];
3xcore2_eHGT_410 h-minBglobin-[heterologous coding sequence]-[post-regulatory elements];
eHGT_363 h-minBglobin-[heterologous coding sequence]-[post-regulatory elements];
eHGT_363 h-minBglobin-[heterologous coding sequence]-[skipping element]-[tag cassette]-10aa-[nuclear localization protein]-[post-regulatory elements];
eHGT_331 h-minBglobin-[heterologous coding sequence]-[post-regulatory elements];
eHGT_332 h-minBglobin-[heterologous coding sequence]-[post-regulatory elements];
eHGT_333 h-minBglobin-[heterologous coding sequence]-[post-regulatory elements];
eHGT_335 h-minBglobin-[heterologous coding sequence]-[post-regulatory elements];
eHGT_334 h-minBglobin-[heterologous coding sequence]-[post-regulatory elements];
eHGT_360 h-minBglobin-[heterologous coding sequence]-[post-regulatory elements];
eHGT_562 h-minBglobin-[heterologous coding sequence]-[post-regulatory elements];
eHGT_563 h-minBglobin-[heterologous coding sequence]-[post-regulatory elements];
eHGT_564 h-minBglobin-[heterologous coding sequence]-[post-regulatory elements];
eHGT_565 h-minBglobin-[heterologous coding sequence]-[post-regulatory elements];
eHGT_566m-minBglobin-[heterologous coding sequence]-[post-regulatory elements];
eHGT_567m-minBglobin-[heterologous coding sequence]-[post-regulatory elements];
eHGT_568m-minBglobin-[heterologous coding sequence]-[post-regulatory elements];
eHGT_569m-minBglobin-[heterologous coding sequence]-[post-regulatory elements];
eHGT_734m-minBglobin-[heterologous coding sequence]-[post-regulatory elements];
eHGT_312 h-minBglobin-[heterologous coding sequence]-[post-regulatory elements];
eHGT_313 h-minBglobin-[heterologous coding sequence]-[post-regulatory elements];
eHGT_377m-minBglobin-[heterologous coding sequence]-[post-regulatory elements];
eHGT_389m-minBglobin-[heterologous coding sequence]-[post-regulatory elements];
eHGT_380 h-minBglobin-[heterologous coding sequence]-[post-regulatory elements];
eHGT_374 h-minBglobin-[heterologous coding sequence]-[post-regulatory elements];
3xcore1_eHGT_387m-minBglobin-[heterologous coding sequence]-[post-regulatory elements];
eHGT_385 h-minBglobin-[heterologous coding sequence]-[post-regulatory elements];
eHGT_386 h-minBglobin-[heterologous coding sequence]-[post-regulatory elements];
eHGT_378m-minBglobin-[heterologous coding sequence]-[post-regulatory elements];
eHGT_642m-minBglobin-[heterologous coding sequence]-[post-regulatory elements];
3xcore2_eHGT_380 h-minBglobin-[heterologous coding sequence]-[post-regulatory elements];
3xcore2_eHGT_374m-minBglobin-[heterologous coding sequence]-[post-regulatory elements];
3xcore2_eHGT_375m-minBglobin-[heterologous coding sequence]-[post-regulatory elements];
eHGT_358 h-minBglobin-[heterologous coding sequence]-[post-regulatory elements];
eHGT_358 h-minBglobin-[heterologous coding sequence]-[skipping element]-[tag cassette]-10aa-[nuclear localization protein]-[post-regulatory elements];
3xcore2_eHGT_390h-[minimal promoter]-[heterologous coding sequence]-[post-regulatory elements];
eHGT_409h-[minimal promoter]-[heterologous coding sequence]-[post-regulatory elements];
eHGT_410h-[minimal promoter]-[heterologous coding sequence]-[post-regulatory elements];
eHGT_361h-[minimal promoter]-[heterologous coding sequence]-[post-regulatory elements];
eHGT_395m-[minimal promoter]-[heterologous coding sequence]-[post-regulatory elements];
eHGT_401h-[minimal promoter]-[heterologous coding sequence]-[post-regulatory elements];
eHGT_391h-[minimal promoter]-[heterologous coding sequence]-[post-regulatory elements];
eHGT_403h-[minimal promoter]-[heterologous coding sequence]-[post-regulatory elements];
3xCore_eHGT_641m-[minimal promoter]-[heterologous coding sequence]-[post-regulatory elements];
3xcore2_eHGT_400m-[minimal promoter]-[heterologous coding sequence]-[post-regulatory elements];
3xcore2_eHGT_401h-[minimal promoter]-[heterologous coding sequence]-[post-regulatory elements];
3xcore2_eHGT_409h-[minimal promoter]-[heterologous coding sequence]-[post-regulatory elements];
3xcore2_eHGT_410h-[minimal promoter]-[heterologous coding sequence]-[post-regulatory elements];
eHGT_363h-[minimal promoter]-[heterologous coding sequence]-[post-regulatory elements];
eHGT_363h-[minimal promoter]-[heterologous coding sequence]-[skipping element]-[tag cassette]-10aa-[nuclear localization protein]-[post-regulatory elements];
eHGT_331h-[minimal promoter]-[heterologous coding sequence]-[post-regulatory elements];
eHGT_332h-[minimal promoter]-[heterologous coding sequence]-[post-regulatory elements];
eHGT_333h-[minimal promoter]-[heterologous coding sequence]-[post-regulatory elements];
eHGT_335h-[minimal promoter]-[heterologous coding sequence]-[post-regulatory elements];
eHGT_334h-[minimal promoter]-[heterologous coding sequence]-[post-regulatory elements];
eHGT_360h-[minimal promoter]-[heterologous coding sequence]-[post-regulatory elements];
eHGT_562h-[minimal promoter]-[heterologous coding sequence]-[post-regulatory elements];
eHGT_563h-[minimal promoter]-[heterologous coding sequence]-[post-regulatory elements];
eHGT_564h-[minimal promoter]-[heterologous coding sequence]-[post-regulatory elements];
eHGT_565h-[minimal promoter]-[heterologous coding sequence]-[post-regulatory elements];
eHGT_566m-[minimal promoter]-[heterologous coding sequence]-[post-regulatory elements];
eHGT_567m-[minimal promoter]-[heterologous coding sequence]-[post-regulatory elements];
eHGT_568m-[minimal promoter]-[heterologous coding sequence]-[post-regulatory elements];
eHGT_569m-[minimal promoter]-[heterologous coding sequence]-[post-regulatory elements];
eHGT_734m-[minimal promoter]-[heterologous coding sequence]-[post-regulatory elements];
eHGT_312h-[minimal promoter]-[heterologous coding sequence]-[post-regulatory elements];
eHGT_313h-[minimal promoter]-[heterologous coding sequence]-[post-regulatory elements];
eHGT_377m-[minimal promoter]-[heterologous coding sequence]-[post-regulatory elements];
eHGT_389m-[minimal promoter]-[heterologous coding sequence]-[post-regulatory elements];
eHGT_380h-[minimal promoter]-[heterologous coding sequence]-[post-regulatory elements];
eHGT_374h-[minimal promoter]-[heterologous coding sequence]-[post-regulatory elements];
3xcore1_eHGT_387m-[minimal promoter]-[heterologous coding sequence]-[post-regulatory elements];
eHGT_385h-[minimal promoter]-[heterologous coding sequence]-[post-regulatory elements];
eHGT_386h-[minimal promoter]-[heterologous coding sequence]-[post-regulatory elements];
eHGT_378m-[minimal promoter]-[heterologous coding sequence]-[post-regulatory elements];
eHGT_642m-[minimal promoter]-[heterologous coding sequence]-[post-regulatory elements];
3xcore2_eHGT_380h-[minimal promoter]-[heterologous coding sequence]-[post-regulatory elements];
3xcore2_eHGT_374m-[minimal promoter]-[heterologous coding sequence]-[post-regulatory elements];
3xcore2_eHGT_375m-[minimal promoter]-[heterologous coding sequence]-[post-regulatory elements];
eHGT_358h-[minimal promoter]-[heterologous coding sequence]-[post-regulatory elements]; or
eHGT_358h-[minimal promoter]-[heterologous coding sequence]-[skipping element]-[tag cassette]-10aa-[nuclear localization protein]-[post-regulatory elements].

50. A vector comprising an artificial expression construct of claim 36.

51. The vector of claim 50, wherein the vector comprises a viral vector.

52. The vector of claim 51, wherein the viral vector comprises a recombinant adeno-associated viral (AAV) vector.

53. An adeno-associated viral (AAV) vector comprising at least one heterologous coding sequence, wherein the heterologous coding sequence is under the transcriptional control of a promoter and an enhancer selected from SEQ ID NO: 48, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 158, SEQ ID NO: SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 40, SEQ ID NO: 41, SEQ ID NO: 42, SEQ ID NO: 43, SEQ ID NO: 44, SEQ ID NO: 52, SEQ ID NO:54, SEQ ID NO: 160, and SEQ ID NO: 157.

54. The AAV vector of claim 53, wherein the heterologous coding sequence encodes an effector element or an expressible element.

55. The AAV vector of claim 54, wherein the effector element comprises a reporter protein or a functional molecule.

56. The AAV vector of claim 55, wherein the reporter protein comprises a fluorescent protein.

57. The AAV vector of claim 55, wherein the functional molecule comprises a functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

58. The AAV vector of claim 54, wherein the expressible element comprises a non-functional molecule.

59. The AAV vector of claim 58, wherein the non-functional molecule comprises a non-functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

60. A transgenic cell comprising an artificial expression construct of claim 36 and/or a vector of claim 53.

61. The transgenic cell of claim 60, wherein the transgenic cell is a non-neuronal cell.

62. The transgenic cell of claim 60, wherein the transgenic cell is a glial cell.

63. The transgenic cell of claim 62, wherein the transgenic cell is an oligodendrocyte, microglia, or astrocyte.

64. The transgenic cell of claim 63, wherein the astrocytes is an L1 interlaminar astrocyte.

65. The transgenic cell of claim 60, wherein the transgenic cell is murine, human, or non-human primate.

66. A non-human transgenic animal comprising an artificial expression construct of claim 36, a vector of claim 53, and/or a transgenic cell of claim 60.

67. The non-human transgenic animal of claim 66, wherein the non-human transgenic animal is a mouse or a non-human primate.

68. An administrable composition comprising an artificial expression construct of claim 36, a vector of claim 53, and/or a transgenic cell of claim 60.

69. A kit comprising an artificial expression construct of claim 36, a vector of claim 53, a transgenic cell of claim 60, and/or a non-human transgenic animal of claim 66.

70. A method for expressing a gene within a targeted population of non-neuronal cells in vivo or in vitro, the method comprising providing the administrable composition of claim 68 in a sufficient dosage and for a sufficient time to a sample or subject comprising the targeted population of non-neuronal cells and other cells thereby expressing the gene within the targeted population of non-neuronal cells.

71. The method of claim 70, wherein the gene encodes an effector element or an expressible element.

72. The method of claim 71, wherein the effector element comprises a reporter protein or a functional molecule.

73. The method of claim 72, wherein the reporter protein comprises a fluorescent protein.

74. The method of claim 72, wherein the functional molecule comprises a functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

75. The method of claim 71, wherein the expressible element comprises a non-functional molecule.

76. The method of claim 75, wherein the non-functional molecule comprises a non-functional ion transporter, enzyme, transcription factor, receptor, membrane protein, cellular trafficking protein, signaling molecule, neurotransmitter, calcium reporter, channelrhodopsin, CRISPR/Cas molecule, editase, guide RNA molecule, microRNA, homologous recombination donor cassette, or DREADD.

77. The method of claim 70, wherein the providing comprises pipetting.

78. The method of claim 77, wherein the pipetting is to a brain slice.

79. The method of claim 78, wherein the brain slice comprises neuronal cells and a targeted population of non-neuronal cells.

80. The method of claim 78, wherein the brain slice comprises a glial cell.

81. The method of claim 80, wherein the glial cell comprises an oligodendrocyte, microglia, or astrocyte.

82. The method of claim 81, wherein the astrocyte comprises an L1 interlaminar astrocyte.

83. The method of claim 78, wherein the brain slice is murine, human, or non-human primate.

84. The method of claim 70, wherein the providing comprises administering to a living subject.

85. The method of claim 84, wherein the living subject is a human, non-human primate, or a mouse.

86. The method of claim 84, wherein the administering to a living subject is through injection.

87. The method of claim 86, wherein the injection comprises intravenous injection, intraparenchymal injection into brain tissue, intracerebroventricular (ICV) injection, intra-cisterna magna (ICM) injection, or intrathecal injection.

88. An artificial expression construct consisting of or consisting essentially of a sequence as set forth in SEQ ID NO: 131, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 134, SEQ ID NO: 161, SEQ ID NO: 162, SEQ ID NO: 163, SEQ ID NO: 164, or SEQ ID NO: 165 or a sequence having at least 90% sequence identity to a sequence as set forth in SEQ ID NO: 131, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 133, SEQ ID NO: 134, SEQ ID NO: 163, SEQ ID NO: 164, or SEQ ID NO: 165.
